(19) United States
(12) Patent Application Publication
(10) Pub. No.: US 2024/0059663 A1
(43) Pub. Date: Feb. 22, 2024

(54) Facile Purification of Cannabinoid Acids

(71) Applicant: Matthew Havis Finley, Novato, CA (US)

(72) Inventor: Matthew Havis Finley, Novato, CA (US)

(21) Appl. No.: 18/268,216

(22) PCT Filed: Dec. 20, 2021
(86) PCT No.: PCT/US21/64468

§ 371 Date: Jun. 17, 2023

Related U.S. Application Data
(60) Provisional application No. 63/127,466, filed on Dec. 18, 2020.

(51) Int. Cl. C07D 311/80

(52) U.S. Cl. CPC C07D 311/80

(57) ABSTRACT

Disclosed are facile methods of purifying a cannabis extract to produce highly purified, crystallized cannabinoid acids, and in particular cannabinoid acids such as THCA, and removing impurities from the same, by using polar aprotic solvents including organic nitrile solvents, such as acetonitrile.

### CROSS-REFERENCE

This application claims priority under 35 U.S.C. § 119(e) to the U.S. Provisional Patent Application entitled “Facile Purification of Cannabinoid Acids,” filed with the U.S. Patent and Trademark Office on Dec. 18, 2020, and assigned Ser. No. 63/127,466, which is incorporated by reference for all purposes as if fully set forth herein.

### TECHNICAL FIELD

The present invention generally relates to the field of cannabinoids, and more specifically to the purification of cannabinoid acids from a cannabis extract.

### BACKGROUND OF THE INVENTION

Cannabis is a genus of flowering plants in the family Cannabaceae commonly recognized as containing the three species C. sativa, C. indica, and C. ruderalis. Cannabis is believed to be one of the first plants to be cultivated, and has a long history of human use for medicinal purposes. Indeed, in what is considered to be the first pharmacopoeia entry, in 2737 BC, the Chinese Emperor Shen-Nung described cannabis as useful to treat over 100 different ailments. Use of cannabis for medicinal purposes continued across the millennia, as was recorded by the Egyptians, Greeks, Romans, and in many other cultures.

Beginning in the 19th century, however, cannabis use became increasingly vilified, and associated only with its psychoactive and intoxicating effects. This eventually culminated in its illegalization and criminalization in most countries. Although extensive use continued, legalization attempts throughout the end of the 20th century repeatedly stalled. To this day, cannabis remains illegal in the United States at the federal level. Nonetheless, two in three Americans support the full legalization of recreational (“adult use”) cannabis, and a majority of U.S. states have now legalized cannabis for medical and/or recreational use.

Despite ongoing federal illegality, cannabis is the most commonly used psychotropic substance in the United States, after alcohol. Based on recent Gallup polls, 55 million Americans identify as active cannabis users, with nearly 25% of young adults aged 18-25 having consumed cannabis in the prior month, over a third in the past year, and over half at some point in their lives. At present, the U.S. legal cannabis industry employs nearly 250,000 Americans, and is worth an estimated $20 billion, with growth of up to $100 billion by 2030.

The compounds generally associated with the effects of cannabis are the secondary plant metabolites known as cannabinoids and terpenoids. Over 100 cannabinoids have been isolated from cannabis, including tetrahydrocannabinol (THC), widely-known because of its intoxicating effects, resulting in the “high” associated with cannabis use, as well as numerous therapeutic and beneficial effects. Besides the principle isomer (−)-trans-Δ9-tetrahydro-cannabinol (Δ9-THC), numerous stereoisomers and double bond isomers exist, e.g., Δ8-THC.

Cannabidiol (CBD), another widely-known cannabinoid, does not produce intoxicating effects, but is purported to have many beneficial properties, including analgesic, antioxidant, anti-inflammatory, antiemetic, anticonvulsant, antipsychotic, anxiolytic, antidepressant, anticompulsive, antitumoral, neuroprotective, and immunomodulatory effects.

Other naturally-occurring cannabinoids found in cannabis include cannabichromene (CBC), cannabigerol (CBG), cannabielsoin (CBE), cannabicyclol (CBL), cannabidinodiol (CBND), cannabicitran (CBT), cannabitriol (CBT), cannabivarin (CBV), cannabigerol monomethyl ether (CBGM), cannabidiphorol (CBDP), tetrahydrocannabiphorol (THCP), and iso-tetrahydrocannabinol (iso-THC) (see, e.g., Grof 2018).

Terpenes (sometimes also “terpenoids”) form another group of compounds found in cannabis, with over 120 identified molecules. Terpenes comprise a class of organic hydrocarbon isoprene polymers constituted by one or more repeating units of the five-carbon building block known as the isoprene unit (i.e., 2-methyl-1,3-butadiene, having the molecular formula C₅H₈). Monoterpenes (e.g., myrcene, d-limonene, α- and β-pinene, terpinolene and linalool) generally dominate the terpene profile of cannabis; sesquiterpenes (e.g., β-caryophyllene and α-humulene) are often also present in cannabis, with other terpenes present in lower amounts (see, e.g., Sommano 2020). Terpenes are known to play a significant role in the fragrance and flavor of cannabis, along with several other compounds, non-limiting examples of which include esters, aldehydes, ketones, flavonoids, and organosulfur compounds.

Although cannabinoids are more popularly understood to be responsible for the mental and physical effects of cannabis, terpenes demonstrate a variety of effects as well, both individually and in combination with cannabinoids, and some evidence suggests a potential “entourage effect” (see, e.g., Russo 2011). Structurally, cannabinoids and terpenes are naturally concentrated in resinous glands called trichomes, which cover the outside of the cannabis plant, and predominant on the flowers and top leaves, although they also may be present in lower concentrations in other plant tissue.

Cannabigerolic acid (CBGA) is the original biogenetic precursor phytocannabinoid and metabolically yields THCA, CBDA, and CBCA via their respective synthases, through enzymatic processes in the plant. That is, THCA synthase and CBDA synthase catalyze oxidative cyclization of the monoterpene moiety of CBGA to form THCA and CBDA, respectively (Taura 2007). Likewise, CBGVA yields THCVA, CBDVA, and CBCVA.

In fresh cannabis plant material, cannabinoids are almost exclusively found in “acidic” form (e.g., THCA, CBDA, CBCA), rather than “neutral” form (e.g., THC, CBD, CBC). Cannabinoids in acidic form have a carboxyl (—COOH) group, which acts as an acid by donating its proton (H⁺) and becoming ionized (to —COO—). Through decarboxylation, the deprotonated carboxyl group is then lost as carbon dioxide (CO₂), leaving the neutral cannabinoid form. By such means, THC and CBD can be obtained from THCA and CBDA.

Acidic forms of cannabinoids possess pharmacological properties mainly at the CB₂ receptor. Generally, they must be converted into their neutral forms through decarboxylation before they are active at the CB₁ receptor and thus have any perceptible “high” (e.g., it is THC, and not THCA, that is responsible for the intoxicating effects of consumed cannabis). How quickly decarboxylation occurs is a function of heat and time. At lower temperatures, such as when a plant is dried and cured after harvesting, decarboxylation occurs slowly and only partially. At higher temperatures, such as when cannabis is combusted by smoking, the cannabinoids are almost instantaneously decarboxylated, and are thus immediately available for absorption through inhalation.

To obtain the intoxicating, therapeutic, and other pharmacological effects of cannabis, its active agents must be administered, for example by ingestion or inhalation. Besides combustion of cannabis flower (i.e., smoking), cannabinoids and/or terpenes may also be obtained in cannabis extracts, which can be produced as liquids, oils, semi-solids, and solids, and subsequently further processed and consumed in a variety of ways.

Extraction, broadly, refers to various processes that can be performed to obtain desired phytochemicals from plants. In the cannabis industry, extraction processes are common, and will be known to those of skill. With cannabis extraction, the ultimate goal is typically to obtain the cannabinoids, terpenes, and other flavor components from the plant matter while leaving behind undesirable waxes, fats, chlorophylls, and other pigments, although extraction processes may be altered to obtain a greater percentage of one or more desired compound(s).

Extraction of cannabinoids from cannabis is generally done using a solvent. Because cannabinoids are lipophilic (i.e., fat-soluble or “hydrophobic”), typical solvents include carbon dioxide, alcohols, or hydrocarbons (all lipophilic solvents, as “like dissolves like”). (“Solventless” extraction techniques also exist, generally using mechanical principles.)

Different solvents and extraction methods have different advantages and drawbacks. Nonetheless, those of skill are generally able to balance the tradeoffs, and select a solvent and extraction method accordingly, depending on the variables to be optimized (e.g., efficiency, cost, characteristics of the desired ultimate product).

Once cannabis is extracted, it often requires further purification and processing to obtain the desired chemical profile. Known methods of obtaining high purity cannabinoid acids from cannabis extracts include chromatographic purification, evaporative crystallization with a nonpolar solvent; low temperature crystallization, at approximately −75° C. with a hydrocarbon solvent; crystallization at reduced temperatures (−40 to 0° C.) with dimethylacetamide, dimethylformamide, or tetrahydrofuran; and extraction with an aqueous base, followed by acidification of the aqueous solution to precipitate the cannabinoid acid.

Each of these known methods has significant disadvantages.

Chromatographic purification, for example, requires the use of costly adsorbents and large quantities of solvents, and therefore has limited ability to scale. Chromatographic purification, most often in the form of high-performance liquid chromatography (HPLC), may, for example, separate components in a mixture through the utilization of pumps that pass a pressurized liquid solvent containing the sample through a column filled with a solid adsorbent material, wherein each component of the sample interacts slightly differently with the adsorbent material, thereby causing varying flow rates for the various components, leading to their separation as they flow out of the column.

Besides HPLC, other types of chromatography including hydrophobic adsorbent chromatography (HAC) and centrifugal partition chromatography (CPC) are both utilized by those of skill, the former using “C18” molecules (hydrocarbons comprised of 18 carbon molecules), and the latter using a liquid stationary and mobile phase, the stationary phase being immobilized by a strong centrifugal force (Foucault, 1995). Both though, have their challenges, including the price of their implementation, as HAC utilizes an expensive stationary phase, and CPC requires a costly machine for operation.

Evaporative crystallization is slow, typically requiring a minimum of 10 days, while producing relatively low yields of the total cannabinoid acids contained within an extract (ranging from about 40% to about 60%), and an impure product, with other unwanted compounds also precipitating. Additionally, it requires large amounts of solvent that is not recovered in the process. Evaporative crystallization generally uses room temperature evaporation of an alkane solvent with a high vapor pressure, causing concentration of solute to increase until it exceeds its solubility; it then slowly precipitates through crystallization.

Low temperature crystallization is also time consuming, requires several days for significant crystallization to occur, and leaves behind a significant portion of the cannabinoid acids in the mother liquor (ranging from about 40% to about 60%), significantly decreasing the overall yield of the process. In addition, decreasing the temperature of the solution supersaturates it, encouraging crystal growth which forces the salt out of solution so that it crystallizes. Low temperature crystallization methods include vacuum cooling crystallization, continuous cooling crystallization, and scraped surface crystallization.

Purification through cannabinoid salt formation creates a waste stream of salt and acid solution and typically uses solvents such as dichloromethane or chloroform which are classified by the U.S. Environmental Protection Agency as probable human carcinogens.

Other methods of cannabinoid acid purification include the use of dimethylacetamide, dimethylformamide, or tetrahydrofuran to grow cannabinoid acid crystals at reduced temperature (see, e.g., U.S. Pat. No. 10,696,646) or the use of short chain hydrocarbons, such as butane, pentane, and hexane; alcohols, including but not limited to methanol, ethanol, propanol, etc.; and terpenes (see, e.g., U.S. App. No. 2017/0008870A1). The requirements of, for example, extensive crystallization times and repeated crystallizations make the first process slow and inefficient. The second process also has known disadvantages, and the extract typically contains impurities that can significantly hinder the ability to yield crystals.

Consequently, a technology that overcomes the above-mentioned disadvantages as well as the other drawbacks of the state of the art, as known to those in the field, is sorely needed. Such a technology, which provides numerous significant improvements over the art as disclosed herein, was invented by Applicant and is described below.

### INCORPORATION BY REFERENCE

Each patent, publication, and non-patent literature cited in the application or in the section entitled References is hereby incorporated by reference in its entirety as if each was incorporated by reference individually. Unless specifically stated otherwise, reference to any document herein is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In some aspects are disclosed methods of facile purification of cannabinoid acids, such methods comprising the steps of (a) combining a cannabis extract and a polar aprotic solvent to form a solution; (b) mixing the solution to form a cannabinoid acid precipitate; and (c) obtaining the cannabinoid acid precipitate.

In some aspects are disclosed methods of facile purification of cannabinoid acids, such methods comprising the steps of (a) combining a cannabis extract and a polar aprotic solvent to form a solution; (b) mixing the solution to form a slurry; (c) filtering the slurry; and (d) obtaining a cannabinoid acid precipitate.

In some embodiments, the polar aprotic solvent used in the methods of the invention is an organic nitrile solvent.

In some embodiments, the organic nitrile solvent is any one or more of acetonitrile, acrylonitrile, benzonitrile, butyronitrile, decanenitrile, pivalonitrile, propionitrile, butyronitrile, isobutyronitrile, malononitrile, and succinonitrile, including mixtures thereof.

In some embodiments, the organic nitrile solvent is acetonitrile.

In some embodiments, the organic nitrile solvent has a melting point below 40° C.

In some embodiments, the organic nitrile solvent has a boiling point below 180° C.

In some embodiments, the organic nitrile solvent has a boiling point between 70° C. and 150° C.

In some embodiments, the organic nitrile solvent has a dielectric constant above 15.0.

In some embodiments, the organic nitrile solvent has a dielectric constant above 37.0.

In some embodiments, the organic nitrile solvent has a density between 0.75 and 1.40 g/cm³.

In some embodiments, the organic nitrile solvent has a dipole moment above 0.1 D.

In some embodiments, the organic nitrile solvent has a dipole moment between 1.8 D and 3.6 D.

In some embodiments, the organic nitrile solvent has a dipole moment between 3.2 D and 3.4 D.

In some embodiments, the cannabis extract was extracted by any of supercritical CO₂, subcritical CO₂, an alcohol, or a hydrocarbon.

In some embodiments, the cannabis extract has an initial purity of less than 60%.

In some embodiments, the cannabis extract has an initial purity of at least 60%.

In some embodiments, the cannabis extract was extracted from an industrial hemp plant material that contained no more than 0.3% THC on a dry-weight basis.

In some aspects, the methods of the invention further comprise the step of purifying the cannabis extract before combining the cannabis extract and the polar aprotic solvent.

In some embodiments, the purification is completed through the use of HPLC, with a column comprising an adsorbent selected from the group consisting of activated carbon, silica gel, alumina, bentonite, and acid activated bleaching clay.

In some embodiments, the purification is completed through the use of winterization or dewaxing.

In some embodiments, the cannabis extract is winterized using the steps comprising: (a) dissolving the cannabis extract in an alcohol solvent; (b) chilling the cannabis extract and the alcohol solvent to between about 0° C. to about −60° C.; (c) filtering the cannabis extract and the alcohol solvent to yield a filtrate; and (d) optionally, distilling the filtrate at a temperature below about 65° C. so as to substantially remove the alcohol solvent.

In some embodiments where the cannabis extract is winterized, the cannabis extract and the alcohol solvent are chilled to between about −5° C. and about −45° C.

In some embodiments, the cannabis extract does not undergo an initial purification step of purifying the cannabis extract before combining the cannabis extract and the polar aprotic solvent.

In some embodiments, the ratio of polar aprotic solvent to cannabis extract is between about 300 mL to about 600 mL of the polar aprotic solvent for every about 1,000 g of cannabis extract.

In some embodiments, the ratio of polar aprotic solvent to cannabis extract is between about 400 mL to about 600 mL of the polar aprotic solvent for every about 1,000 g of cannabis extract.

In some embodiments, the ratio of polar aprotic solvent to cannabis extract is between about 350 mL to about 450 mL of the polar aprotic solvent for every about 1,000 g of cannabis extract.

In some aspects, the methods of the invention further comprise the step of evaporating residual solvent.

In some embodiments, the residual solvent is evaporated through the use of ambient evaporation.

In some embodiments, the ambient evaporation occurs at temperatures between about 15° C. to about 35° C.

In some embodiments, the residual solvent is evaporated through the use of a vacuum oven.

In some embodiments, the vacuum oven is heated to between about 25° C. to about 45° C., and maintained at a pressure of less than about 200 torr.

In some embodiments, the residual solvent is evaporated through the use of a rotary evaporator and a water bath.

In some embodiments, the water bath is heated to a temperature of between about 65° C. and 70° C., has a beginning pressure of between about 400 torr to about 500 torr, and has a final pressure of between about 5 torr to about 30 torr.

In some embodiments, the filtering step of the methods of the invention is performed using any of gravity filtration, vacuum filtration, pressure filtration, and centrifugal filtration.

In some embodiments, the filtering step is performed using a filter having a first filter screen and a second filter screen, wherein the first filter screen contains a cut size of between about 110 microns to about 140 microns, and the second filter screen contains a pore size of between about 70 microns to about 100 microns.

In some embodiments, the mixing step of the methods of the invention is performed by a magnetic stir bar or high-shear blade. In some embodiments, the magnetic stir bar or high-shear blade operates at a stirring speed of between about 300 rpm to about 1,500 rpm.

In some embodiments, the mixing step of the methods of the invention is performed until the slurry is substantially homogenous. In some embodiments, the mixing is performed until the cannabinoid acid precipitate has substantially ceased precipitating. In some embodiments, the mixing is performed by agitating the solution until the slurry is substantially homogenous, and the cannabinoid acid precipitate has substantially ceased precipitating.

In some embodiments, the mixing is performed for between about 10 minutes to about 36 hours. In some embodiments, the mixing is performed for between about 2 hours to about 24 hours. In some embodiments, the mixing is performed for between about 6 hours to about 18 hours. In some embodiments, the mixing is performed at a temperature of between about 40° C. to about 80° C.

In some embodiments, the cannabinoid acid precipitate of the invention comprises at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the cannabinoid acid precipitate has a purity of at least 99% cannabinoid acids. In some embodiments, the cannabinoid acid precipitate comprises THCA. In some embodiments, the cannabinoid acid precipitate has a purity of at least 99% THCA. In some embodiments, cannabinoid acid precipitate has no more than 5% THCA.

In some aspects, the methods of the invention further comprise the step of decarboxylating the cannabinoid acid precipitate. In some embodiments, decarboxylation is performed by heating the cannabinoid acid precipitate to a temperature of between about 70° C. to about 200° C. In some embodiments, the cannabinoid acid precipitate is heated to a temperature of between about 85° C. to about 150° C. under ambient pressure. In some embodiments, the cannabinoid acid precipitate is heated to a temperature of between about 70° C. to about 120° C. under vacuum.

In some aspects are disclosed cannabinoid acid precipitates having a purity of at least 99% cannabinoid acids, wherein the cannabinoid acid precipitate is prepared according to the method of the invention.

In some embodiments, the cannabinoid acid precipitate has a purity of at least 99% THCA. In some embodiments, the cannabinoid acid precipitate has no more than 5% THCA.

In some embodiments, the methods of the invention further comprise the steps of. (a) collecting a mother liquor in a container when filtering the slurry; (b) cooling the mother liquor to between about −10° C. to about −40° C.; (c) waiting until any dark pigments settle in the container; and (d) pouring out the mother liquor, to obtain a lighted solution.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be also realized that such equivalent structures do not depart from the spirit and scope of the invention as set forth in the appended claims. Hence, this summary has been made with the understanding that it is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled.

### BRIEF SUMMARY OF THE DRAWINGS

To further clarify various aspects of the invention, a more particular description of the invention will be rendered by reference to certain exemplary embodiments thereof which are illustrated in the included figures. It should be understood and appreciated that the figures depict only illustrated embodiments of the invention and are therefore not to be considered limiting of its scope. They are simply provided as exemplary illustrations of certain concepts of some embodiments of the invention.

Certain aspects of the invention are therefore further described and explained with additional specificity and detail, but still by way of example only, with reference to the accompanying figures in which:

FIG. 1 is a method of facile purification of cannabinoid acids, according to an embodiment of the invention.

FIG. 2 is a method of removing plant lipids from the initial extract solution via winterization, according to an embodiment of the invention.

FIG. 3 is a method of facile purification of cannabinoids comprising an additional step of decarboxylating the cannabinoid acid precipitate to produce a neutral cannabinoid product, according to an embodiment of the invention.

FIG. 4 is a method used to determine whether initial purification of the cannabis extract should be performed prior to continuing to subsequent steps of the method of facile purification of cannabinoid acids of the invention, according to some embodiments.

FIG. 5 is a method of facile purification of cannabinoid acids according to one aspect of the invention.

FIG. 6 is a method of facile purification of cannabinoid acids according to one aspect of the invention.

### DETAILED DESCRIPTION

While various aspects and features of certain embodiments have been summarized above, the following detailed description illustrates several exemplary embodiments in further detail to enable one of skill in the art to practice such embodiments, and to make and use the full scope of the invention claimed. The described examples are provided for illustrative purposes and are not intended to limit the scope of the invention or its applications. It will be understood that many modifications, substitutions, changes, and variations in the described examples, embodiments, applications, and details of the invention illustrated herein can be made by those skilled in the art without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and novel features disclosed. The description below is designed to make such embodiments apparent to a person of ordinary skill, in that the embodiments shall be both readily cognizable and readily creatable without undue experimentation, solely using the teachings herein together with general knowledge of the art.

As used in this specification and the appended claims, the singular forms “a,” “an,” and “the” include plural referents unless the context clearly dictates otherwise. Thus, for example, reference to “an extraction system” includes reference to a combination of two or more extraction systems, and reference to “a cannabinoid” includes reference to a combination of two or more cannabinoids. While the term “one or more” may be used, its absence (or its replacement by the singular) does not signify the singular only, but simply underscores the possibility of multiple agents or ingredients in particular embodiments. The word “exemplary” is used herein to mean “serving as an example, instance, or illustration.” Any aspect, embodiment, process, or implementation described herein as “exemplary” is therefore not to be construed as necessarily preferred or advantageous over others.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

Unless otherwise indicated, all numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the description and claims are approximations that can vary depending upon the desired properties sought to be obtained by a particular embodiment. In some embodiments, “about” refers to plus or minus five percent (+5%) of the recited unit of measure. The term “substantially,” where it is applied to modify a parameter or characteristic herein, will be read in the context of the invention and in light of the knowledge in the art to provide certainty, e.g., by using a standard that is recognized in the art for measuring the meaning of substantially as a term of degree, or by ascertaining the scope as would one of skill in the art.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless otherwise provided or implied, the temperature of a process or process step is presumed to be ambient temperature (e.g., room temperature); likewise, unless otherwise provided or implied, the pressure during a process or process step is presumed to be ambient pressure (e.g., atmospheric pressure at sea level); as will be appreciated by those of skill.

Unless defined otherwise, all technical and scientific terms herein have the meaning as commonly understood by one of ordinary skill in the art to which this invention belongs. Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only and is not intended to be limiting.

Generally, the nomenclature used and procedures performed herein are those known in field(s) relating to that of one or more aspects of the invention, and are those that will be well-known and commonly employed in such field(s). Standard techniques and procedures will be those generally performed according to conventional methods in the art.

Among the various aspects of the invention is a purification method for cannabis extracts comprising cannabinoid acids, and especially for extracts rich in cannabinoid acids, such as CBCA (cannabichromenenic acid), CBCVA (cannabichromevarinic acid), CBDA (cannabidiolic acid), CBDMA (cannabidiolic acid monomethyl ether), CBDPA (cannabidiphorolic acid), CBDVA (cannabidivarinic acid), CBFA (cannabifuranic acid), CBEA (cannabielsoinic acid), CBGA (cannabigerolic acid), CBGVA (cannabigerovarinic acid), CBLA (cannabicyclolic acid), CBNA (cannabinolic acid), CBNDA (cannabinodiolic acid), CBTA (cannabitriolic acid), THCA (tetrahydrocannabinolic acid), THCPA (tetrahydrocannabipgorolic acid), and THCVA (tetrahydrocanabivarinic acid).

In some embodiments, “cannabis extract” refers to an extract of cannabis plant material obtained through the use of a solvent capable of dissolving cannabis compounds. In some embodiments, a cannabis extract refers to the product of a cannabis extraction process and includes, at least one of, cannabinoid acids, neutral cannabinoids, terpenoids, flavonoids, ketones, aldehydes, esters and organosulfur compounds. In preferred embodiments, a cannabis extract has a relatively high content of cannabinoid acids and a relatively low content of neutral cannabinoids. In some embodiments, a cannabis extract is the product of a cannabis extraction process wherein a solvent is added to a cannabis plant material, wherein the solvent is capable of dissolving desired compounds from the cannabis plant material into a solution, from which is obtained the cannabis extract, e.g., a crude cannabis oil, from which may also be obtained a refined cannabis oil, and both crude and refined oils shall be understood to be cannabis extracts in some embodiments of the invention. In some preferred embodiments, a cannabis extract is the product of a cannabis extraction process that results in a low amount of decarboxylation of cannabinoid acids into neutral cannabinoids. In some preferred embodiments, a cannabis extract is the product of a cannabis extraction process using a cannabis plant material starting product with a low amount of neutral cannabinoids relative to cannabinoid acids, e.g., that had a low amount of decarboxylation of cannabinoid acids into neutral cannabinoids. Cannabis extracts can be obtained as liquids, oils, semi-solids, and solids, and using solvents that include subcritical or supercritical CO₂ (e.g., as CO₂ oil, CO₂ hash oil, or CO₂ cannabis oil) an alcohol (e.g., ethanol hash oil or EHO), an ether (e.g., dimethyl ether) and/or a hydrocarbon (e.g., butane hash oil or BHO). For example, supercritical CO₂ extraction techniques are disclosed in, e.g., U.S. Pat. Nos. 10,307,447; 10,092,852; 9,744,200; 9,649,349; 8,895,078; 8,846,409; 10,625,176, 6,403,126. Subcritical CO₂ extraction techniques are disclosed in, e.g., U.S. Pat. No. 10,688,410. Ethanol and other alcohol extraction techniques are disclosed in, e.g., U.S. Pat. Nos. 10,414,709; 10,413,845; 10,406,453; U.S. Pub. Nos. 2017/0333503; 2003/0017216; 2016/0038437. Hydrocarbon extraction techniques are disclosed in, e.g., U.S. Pat. Nos. 9,144,751; 9,145,532; 9,587,203; 9,682,333; 9,789,147; 9,926,512; 10,329,513; U.S. Pub. Nos. 2017/0113161; 2019/0374873.

In some embodiments, “cannabinoid acids” or “acidic cannabinoids” refer generally to a heterogenous group of naturally-occurring compounds found within the cannabis plant that are the carboxylic acid precursors of their corresponding “neutral” cannabinoid (see, e.g., Burstein 2014). Non-limiting examples of cannabinoid acids include, as mentioned, CBCA (cannabichromenenic acid), CBCVA (cannabichromevarinic acid), CBDA (cannabidiolic acid), CBDMA (cannabidiolic acid monomethyl ether), CBDPA (cannabidiphorolic acid), CBDVA (cannabidivarinic acid), CBFA (cannabifuranic acid), CBEA (cannabielsoinic acid), CBGA (cannabigerolic acid), CBGVA (cannabigerovarinic acid), CBLA (cannabicyclolic acid), CBNA (cannabinolic acid), CBNDA (cannabinodiolic acid), CBTA (cannabitriolic acid), THCA (tetrahydrocannabinolic acid, including Δ8 and Δ9 THCA), THCPA (tetrahydrocannabipgorolic acid), and THCVA (tetrahydrocanabivarinic acid). “Neutral cannabinoids” refer to the decarboxylated form of a given cannabinoid acid; i.e., the cannabinoid where the additional carboxyl group is removed. Without being bound by theory, decarboxylation generally is a function of pressure, temperature, and time, and typically occurs when a given cannabinoid acid is exposed to heat.

As it relates to the invention, where such inclusion is technically meaningful, each cannabinoid acid listed herein, i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, will be understood to include their isomers, such as structural isomers and stereoisomers (including enantiomers), the −A and −B isomers for each cannabinoid acid, double bond isomers, and other such isomers known to those of skill. Thus, “THCA” will, in some embodiments, be understood to include THCA-A and THCA-B. In some embodiments, included among cannabinoids are the cannabinoid carboxylic acids and their carboxylate salts (see, e.g., U.S. Pat. No. 9,376,367). Accordingly, reference to a cannabis extract includes, in such embodiments, cannabinoid carboxylic acid-rich extracts and other extracts containing cannabinoid carboxylic acids, as well as the crystalline salts of cannabinoid carboxylic acids.

Additionally, reference to a given cannabinoid acid includes the various alkyl chain lengths associated with the given cannabinoid acid, as illustrated by Cₙ, wherein “n” refers to the number of carbon atoms in each alkyl chain. Thus, “THCA” also includes THCA-C₁, THCA-C₂, THCA-C₃, THCA-C₄, THCA-C₅, THCA-C₆, and THCA-C₇. Further, reference to a given cannabinoid acid will include all possible isomers, such as but not limited to its −A and −B isomers, and all possible combinations of alkyl chain lengths, including chains comprised of 1, 2, 3, 4, 5, 6, or 7 carbon atoms. So, herein, reference to “THCA” will include THCA-C₁ A, THCA-C₁ B, THCA-C₂ A, THCA-C₂ B, THCA-C₃ A, THCA-C₃ B, THCA-C₄ A, THCA-C₄ B, THCA-C₅ A, THCA-C₅ B, THCA-C₆ A, THCA-C₆ B, and THCA-C₇ A, and THCA-C₇ B. As with THCA, the other cannabinoid acids disclosed herein, e.g., CBCA, CBCVA, CBDA, CBDMA, CBDPA, CBDVA, CBEA, CBFA, CBGA, CBGVA, CBLA, CBNA, CBNDA, CBTA, THCPA, and THCVA. Thus, any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, will also, in some embodiments, include isomers, such as -A and -B isomers, and alkyl chains C₁-C₇, i.e., C₁, C₂, C₃, C₄, C₅, C₆, and C₇.

In some embodiments, each cannabinoid acid listed herein, i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA may further comprise one or more chemical moieties including, but not limited to, methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like, and still be within the spirit of the invention as disclosed herein.

Cannabis extracts useful in the invention will include at least one cannabinoid acid, wherein the at least one cannabinoid acid comprises at least about 0.0001% of the total cannabinoid content of the cannabis extract. In some embodiments, cannabis extracts useful in the invention will include at least one cannabinoid acid wherein the at least one cannabinoid acid comprises at least about 0.0001%, at least about 0.001%, at least about 0.01%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, or at least about 80% of the total cannabinoid content of the cannabis extract. In some embodiments, a cannabis extracts useful in the invention will include at least one cannabinoid acid, wherein the at least one cannabinoid acid comprises at least about 80% of the total cannabinoid content of the cannabis extract, including about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.9%, about or about 99.99% of the total cannabinoid content of the cannabis extract.

In some embodiments, the total cannabinoid acids in the cannabis extract is an amount of between about 10% and about 95%, about 20% and about 95%, between about 30% and about 95%, between about 40% and about 95%, between about 50% and about 95%, between about 60% and about 95%, between about 70% and about 95%, between about 80% and about 95%, or between about 90% and about 95%. In some embodiments, the total cannabinoid acids in the cannabis extract is an amount of between about 10% and about 90%, about 20% and about 90%, between about 30% and about 90%, between about 40% and about 90%, between about 50% and about 90%, between about 60% and about 90%, between about 70% and about 90%, or between about 80% and about 90%. In some embodiments, the total cannabinoid acids in the cannabis extract is an amount of between about 10% and about 85%, about 20% and about 85%, between about 30% and about 85%, between about 40% and about 85%, between about 50% and about 85%, between about 60% and about 85%, between about 70% and about 85%, or between about 80% and about 85%. In some embodiments, the total cannabinoid acids in the cannabis extract is an amount of between about 10% and about 80%, about 20% and about 80%, between about 30% and about 80%, between about 40% and about 80%, between about 50% and about 80%, between about 60% and about 80%, or between about 70% and about 80%. In some embodiments, the total cannabinoid acids in the cannabis extract is an amount of between about 10% and about 75%, about 20% and about 75%, between about 30% and about 75%, between about 40% and about 75%, between about 50% and about 75%, between about 60% and about 75%, or between about 70% and about 75%. In some embodiments, the total cannabinoid acids in the cannabis extract is an amount of between about 10% and about 70%, about 20% and about 70%, between about 30% and about 70%, between about 40% and about 70%, between about 50% and about 70%, or between about 60% and about 70%.

In some embodiments, the amount of an individual cannabinoid acid in an extract may be determined through the use of high-performance liquid chromatography (HPLC), wherein a C18 column (an octyldecylsilane containing 18 carbons bound to silica), or an equivalent adsorbent, as would be apparent to one of skill, is used as the adsorbent, and the system operates at a pressure of between about (5,000-35,000 kPa), including about 5,000 kPa, about 5,500 kPa, about 6,000 kPa, about 6,500 kPa, about 7,000 kPa, about 7,500 kPa, about 8,000 kPa, about 8,500 kPa, about 9,000 kPa, about 9,500 kPa, about 10,000 kPa, about 10,500 kPa, about 11,000 kPa, about 11,500 kPa, about 12,000 kPa, about 12,500 kPa, about 13,000 kPa, about 13,500 kPa, about 14,000 kPa, about 14,500 kPa, about 15,000 kPa, about 15,500 kPa, about 16,000 kPa, about 16,500 kPa, about 17,000 kPa, about 17,500 kPa, about 18,000 kPa, about 18,500 kPa, about 19,000 kPa, about 19,500 kPa, about 20,000 kPa, about 20,500 kPa, about 21,000 kPa, about 21,500 kPa, about 22,000 kPa, about 22,500 kPa, about 23,000 kPa, about 23,500 kPa, about 24,000 kPa, about 24,500 kPa, about 25,000 kPa, about 25,500 kPa, about 26,000 kPa, about 26,500 kPa, about 27,000 kPa, about 27,500 kPa, about 28,000 kPa, about 28,500 kPa, about 29,000 kPa, about 29,500 kPa, about 30,000 kPa, about 30,500 kPa, about 31,000 kPa, about 31,500 kPa, about 32,000 kPa, about 32,500 kPa, about 33,000 kPa, about 33,500 kPa, about 34,000 kPa, about 34,500 kPa, about 35,000 and values in between, as would be apparent to one of skill; and has a column between about 2.1 mm and about 4.6 mm in diameter, including about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, and values in between as would be apparent to one of skill; and about 30 mm to about 250 mm in length, including about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, about 175 mm, about 180 mm, about 185 mm, about 190 mm, about 195 mm, about 200 mm, about 205 mm, about 210 mm, about 215 mm, about 220 mm, about 225 mm, about 230 mm, about 235 mm, about 240 mm, about 245 mm, about 250 mm, and values in between, as apparent to one of skill (Develosil, 2019; Gerber et al., 2004).

In some embodiments, a cannabis extract includes a first cannabinoid acid comprising between about 0.0001% to about 100% of the total cannabinoid content of the cannabis extract. In some embodiments, a cannabis extract includes a first cannabinoid acid comprising between about 0.0001% to about 100% of the total cannabinoid content of the cannabis extract, and a second cannabinoid acid comprising between about 0.0001% to about 100% of the total cannabinoid content of the cannabis extract. In some embodiments, a cannabis extract includes a first cannabinoid acid and a second cannabinoid acid together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, and a third cannabinoid acid comprising between about 0.0001% to about 100% of the total cannabinoid content of the cannabis extract. In some embodiments, a cannabis extract includes a first cannabinoid acid, a second cannabinoid acid, and a third cannabinoid acid together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, and a fourth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, and a fourth cannabinoid acid, together comprising between about 0.00010% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, and a fifth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, and a fifth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, and a sixth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, and a sixth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, and a seventh cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, and a seventh cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, and an eighth cannabinoid acid comprising between about 0.00010% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, and an eighth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, and a ninth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, and a ninth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, and a tenth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, and a tenth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, and an eleventh cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, and an eleventh cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, and a twelfth cannabinoid acid comprising between about 0.00010% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, and a twelfth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, a twelfth cannabinoid acid comprising between about 0.00010% to about 100% of the total cannabinoid content of the cannabis extract, and a thirteenth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, and a thirteenth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, a twelfth cannabinoid acid comprising between about 0.00010% to about 100% of the total cannabinoid content of the cannabis extract, a thirteenth cannabinoid acid comprising between about 0.0001% to about 100% of the total cannabinoid content of the cannabis extract, and a fourteenth cannabinoid acid comprising between about 0.00010% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, and a fourteenth cannabinoid acid, together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, a twelfth cannabinoid acid comprising between about 0.00010% to about 100% of the total cannabinoid content of the cannabis extract, a thirteenth cannabinoid acid comprising between about 0.0001% to about 100% of the total cannabinoid content of the cannabis extract, a fourteenth cannabinoid acid comprising between about 0.0001% to about 100% of the total cannabinoid content of the cannabis extract, and a fifteenth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, and a fifteenth cannabinoid acid together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, a twelfth cannabinoid acid comprising between about 0.0001% to about 100% of the total cannabinoid content of the cannabis extract, a thirteenth cannabinoid acid comprising between about 0.0001% to about 100% of the total cannabinoid content of the cannabis extract, a fourteenth cannabinoid acid comprising between about 0.0001% to about 100% of the total cannabinoid content of the cannabis extract, a fifteenth cannabinoid acid comprising between about 0.0001% to about 100% of the total cannabinoid content of the cannabis extract, and a sixteenth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, a fifteenth cannabinoid acid, and a sixteenth cannabinoid acid together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, a second cannabinoid acid comprising between about 0.0001% to about 100% of the total cannabinoid content of the cannabis extract, a third cannabinoid acid comprising between about 0.0001% to about 100% of the total cannabinoid content of the cannabis extract, a fourth cannabinoid acid comprising between about 0.0001% to about 100% of the total cannabinoid content of the cannabis extract, a fifth cannabinoid acid comprising between about 0.0001% to about 100% of the total cannabinoid content of the cannabis extract, a sixth cannabinoid acid comprising between about 0.0001% to about 100% of the total cannabinoid content of the cannabis extract, a seventh cannabinoid acid comprising between about 0.0001% to about 100% of the total cannabinoid content of the cannabis extract, an eighth cannabinoid acid comprising between about 0.0001% to about 100% of the total cannabinoid content of the cannabis extract, a ninth cannabinoid acid comprising between about 0.0001% to about 100% of the total cannabinoid content of the cannabis extract, a tenth cannabinoid acid comprising between about 0.0001% to about 100% of the total cannabinoid content of the cannabis extract, an eleventh cannabinoid acid comprising between about 0.0001% to about 100% of the total cannabinoid content of the cannabis extract, a twelfth cannabinoid acid comprising between about 0.00010% to about 100% of the total cannabinoid content of the cannabis extract, a thirteenth cannabinoid acid comprising between about 0.0001% to about 100% of the total cannabinoid content of the cannabis extract, a fourteenth cannabinoid acid comprising between about 0.0001% to about 100% of the total cannabinoid content of the cannabis extract, a fifteenth cannabinoid acid comprising between about 0.0001% to about 100% of the total cannabinoid content of the cannabis extract, a sixteenth cannabinoid acid comprising between about 0.0001% to about 100% of the total cannabinoid content of the cannabis extract, and a seventeenth cannabinoid acid comprising between about 0.0001% to about 100% of the total cannabinoid content of the cannabis extract. In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, a fifteenth cannabinoid acid, a sixteenth cannabinoid acid, and a seventeenth cannabinoid acid together comprising between about 0.0001% to about 100% of the total cannabinoid content of the cannabis extract.

In some embodiments, a cannabis extract includes a first cannabinoid acid comprising between about 0.0001% to about 100% of the total cannabinoid content of the cannabis extract, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, a cannabis extract includes a first cannabinoid acid and a second cannabinoid acid, together comprising between about 0.0001% to about 100% of the total cannabinoid content of the cannabis extract, wherein the first cannabinoid acid and second cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, and a third cannabinoid acid, together comprising between about 0.0001% to about 100% of the total cannabinoid content of the cannabis extract, wherein the first cannabinoid acid, the second cannabinoid acid, and the third cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, and a fourth cannabinoid acid, together comprising between about 0.0001% to about 100% of the total cannabinoid content of the cannabis extract, wherein the first cannabinoid acid, the second cannabinoid acid, the third cannabinoid acid, and the fourth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, and a fifth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, and the fifth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, and a sixth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, and the sixth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, and a seventh cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, and the seventh cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, and an eighth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, and the eighth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, and a ninth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, and the ninth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, and a tenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, and the tenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, and an eleventh cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, and the eleventh cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, and a twelfth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, and the twelfth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, and a thirteenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, the twelfth cannabinoid acid, and the thirteenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, and a fourteenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, the twelfth cannabinoid acid, the thirteenth cannabinoid acid, and the fourteenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, and a fifteenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, the twelfth cannabinoid acid, the thirteenth cannabinoid acid, the fourteenth cannabinoid acid, and the fifteenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, a fifteenth cannabinoid acid, and a sixteenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, the twelfth cannabinoid acid, the thirteenth cannabinoid acid, the fourteenth cannabinoid acid, the fifteenth cannabinoid acid, and the sixteenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes a first cannabinoid acid, a second cannabinoid acid, a third cannabinoid acid, a fourth cannabinoid acid, a fifth cannabinoid acid, a sixth cannabinoid acid, a seventh cannabinoid acid, an eighth cannabinoid acid, a ninth cannabinoid acid, a tenth cannabinoid acid, an eleventh cannabinoid acid, a twelfth cannabinoid acid, a thirteenth cannabinoid acid, a fourteenth cannabinoid acid, a fifteenth cannabinoid acid, a sixteenth cannabinoid acid, and a seventeenth cannabinoid acid, together comprising between about 0.0001% to about 100% of the total cannabinoid content of the cannabis extract, wherein the first cannabinoid acid, the second cannabinoid acid, the third cannabinoid acid, the fourth cannabinoid acid, the fifth cannabinoid acid, the sixth cannabinoid acid, the seventh cannabinoid acid, the eighth cannabinoid acid, the ninth cannabinoid acid, the tenth cannabinoid acid, the eleventh cannabinoid acid, the twelfth cannabinoid acid, the thirteenth cannabinoid acid, the fourteenth cannabinoid acid, the fifteenth cannabinoid acid, the sixteenth cannabinoid acid, and the seventeenth cannabinoid acid are each any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, but are not the same cannabinoid acid.

In some embodiments, a cannabis extract includes one cannabinoid acid, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and comprises between at least about 0.0001% to about 100% of the total cannabinoid content of the cannabis extract. In some embodiments, a cannabis extract includes one cannabinoid acid, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and comprises at least about 0.0001%, at least about 0.001%, at least about 0.01%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total cannabinoid content of the cannabis extract.

In some embodiments, a cannabis extract includes more than one (e.g. two, three, four, five, six, seven, eight, nine, ten, eleven, twelve, thirteen, fourteen, fifteen, sixteen, seventeen, or more than seventeen) cannabinoid acids, wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and the sum of the concentrations of the more than one cannabinoid acids comprises at least about 0.0001%, at least about 0.001%, at least about 0.01%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total cannabinoid content of the cannabis extract.

Herein, cannabis extracts “rich” in cannabinoid acids will possess cannabinoid acids in a concentration that, either alone or in combination, comprises at least about 50% of the total composition of the cannabis extract, including at least about 510%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total composition of the cannabis extract.

Thus, a cannabis extract comprising a single cannabinoid acid is considered “rich” if the cannabinoid acid is present at a concentration that is greater than about 50% of the total composition of the cannabis extract; whereas a cannabis extract containing more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids will have a total cannabinoid acid concentration (i.e., the sum of the concentrations of each cannabinoid acid) that is greater than about 50% of the total composition of the cannabis extract.

In some embodiments, the cannabis extract is rich in at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA. In some embodiments, the cannabis extract is rich in THCA. In some embodiments, the cannabis extract is rich in THVCA. In some embodiments, the cannabis extract is rich in CBCA. In some embodiments, the cannabis extract is rich in CBCVA. In some embodiments, the cannabis extract is rich in CBDA. In some embodiments, the cannabis extract is rich in CBDVA. In some embodiments, the cannabis extract is rich in CBGA. In some embodiments, the cannabis extract is rich in CBGVA. In some embodiments, the cannabis extract is rich in CBDMA. In some embodiments, the cannabis extract is rich in CBDPA. In some embodiments, the cannabis extract is rich in CBEA. In some embodiments, the cannabis extract is rich in CBFA. In some embodiments, the cannabis extract is rich in CBLA. In some embodiments, the cannabis extract is rich in CBNA. In some embodiments, the cannabis extract is rich in CBNDA. In some embodiments, the cannabis extract is rich in CBTA. In some embodiments, the cannabis extract is rich in THCPA.

In some embodiments, the cannabis extract is rich in more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids, wherein the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA. Meaning, in some embodiments, the cannabis extract is rich in THCA; in some embodiments, the cannabis extract is rich in THCA and THCVA; in some embodiments, the cannabis extract is rich in THCA, THCVA and CBCA; in some embodiments, the cannabis extract is rich in THCA, THCVA, CBCA, and CBCVA; in some embodiments, the cannabis extract is rich in THCA, THCVA, CBCA, CBCVA, and CBDA; in some embodiments, the cannabis extract is rich in THCA, THCVA, CBCA, CBCVA, CBDA, and CBDVA; in some embodiments, the cannabis extract is rich in THCA, THCVA, CBCA, CBCVA, CBDA, CBDVA, and CBGA; in some embodiments, the cannabis extract is rich in THCA, THCVA, CBCA, CBCVA, CBDA, CBDVA, CBGA, and CBGVA; in some embodiments, the cannabis extract is rich in THCA, THCVA, CBCA, CBCVA, CBDA, CBDVA, CBGA, CBGVA, and CBDMA; in some embodiments, the cannabis extract is rich in THCA, THCVA, CBCA, CBCVA, CBDA, CBDVA, CBGA, CBGVA, CBDMA, and CBDPA; in some embodiments, the cannabis extract is rich in THCA, THCVA, CBCA, CBCVA, CBDA, CBDVA, CBGA, CBGVA, CBDMA, CBDPA, and CBEA; in some embodiments, the cannabis extract is rich in THCA, THCVA, CBCA, CBCVA, CBDA, CBDVA, CBGA, CBGVA, CBDMA, CBDPA, CBEA, and CBFA; in some embodiments, the cannabis extract is rich in THCA, THCVA, CBCA, CBCVA, CBDA, CBDVA, CBGA, CBGVA, CBDMA, CBDPA, CBEA, CBFA, and CBLA; in some embodiments, the cannabis extract is rich in THCA, THCVA, CBCA, CBCVA, CBDA, CBDVA, CBGA, CBGVA, CBDMA, CBDPA, CBEA, CBFA, CBLA, and CBNA; in some embodiments, the cannabis extract is rich in THCA, THCVA, CBCA, CBCVA, CBDA, CBDVA, CBGA, CBGVA, CBDMA, CBDPA, CBEA, CBFA, CBLA, CBNA, and CBNDA; in some embodiments, the cannabis extract is rich in THCA, THCVA, CBCA, CBCVA, CBDA, CBDVA, CBGA, CBGVA, CBDMA, CBDPA, CBEA, CBFA, CBLA, CBNA, CBNDA, and CBTA; in some embodiments, the cannabis extract is rich in THCA, THCVA, CBCA, CBCVA, CBDA, CBDVA, CBGA, CBGVA, CBDMA, CBDPA, CBEA, CBFA, CBLA, CBNA, CBNDA, CBTA, and THCPA; or, in some embodiments, the cannabis extract is rich in any other possible two, three, four, five, six, seven, eight, nine, ten, eleven, twelve, thirteen, fourteen, fifteen, sixteen or seventeen-member combination one could make from the cannabinoid acids disclosed herein, (i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA) and/or other cannabinoid acids known to those of skill, as would be appreciated by those of skill.

In some embodiments, the cannabis extract is rich in at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and is additionally rich in other cannabinoid acids known to those of skill.

In some embodiments, a cannabis extract rich in cannabinoid acids includes one cannabinoid acid, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and comprises at least about 50% of the total composition of the cannabis extract. In some embodiments, a cannabis extract rich in cannabinoid acids includes one cannabinoid acid, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and comprises at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total composition of the cannabis extract.

In some embodiments, a cannabis extract rich in cannabinoid acids includes more than one (e.g. two, three, four, five, six, seven, eight, nine, ten, eleven, twelve, thirteen, fourteen, fifteen, sixteen, seventeen, or more than seventeen) cannabinoid acids, wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and the sum of the concentrations of the more than one cannabinoid acids comprises at least about 50% of the total composition of the cannabis extract. In some embodiments, a cannabis extract rich in cannabinoid acids includes more than one (e.g. two, three, four, five, six, seven, eight, nine, ten, eleven, twelve, thirteen, fourteen, fifteen, sixteen, seventeen, or more than seventeen) cannabinoid acids, wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and the sum of the concentrations of the more than one cannabinoid acids comprises at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total composition of the cannabis extract.

In some embodiments, an extract may have a cannabinoid acid content characterized as “poor,” wherein the total cannabinoid acid content, i.e. the sum of all cannabinoid acid concentrations, comprises less than 50% of the total composition of the cannabis extract. Necessarily, then, in embodiments wherein an extract characterized as “poor” is chosen only containing a single cannabinoid acid, that cannabinoid acid will also comprise less than 50% of the total composition of the cannabis extract.

Thus, in some embodiments, a cannabis extract characterized as “poor” includes one cannabinoid acid, wherein the cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and comprises less than about 50%, less than about 49.9%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, less than about 0.1%, less than about 0.01%, less than about 0.001%, or less than about 0.0001% of the total composition of the cannabis extract.

Additionally, in some embodiments, a cannabis extract characterized as “poor” includes more than one (e.g. two, three, four, five, six, seven, eight, nine, ten, eleven, twelve, thirteen, fourteen, fifteen, sixteen, seventeen, or more than seventeen) cannabinoid acids, wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA, and the sum of the total concentrations of the more than one cannabinoid acids comprises less than about 50%, less than about 49.9%, less than about 49%, less than about 48%, less than about 47%, less than about 46%, less than about 45%, less than about 44%, less than about 43%, less than about 42%, less than about 41%, less than about 40%, less than about 39%, less than about 38%, less than about 37%, less than about 36%, less than about 35%, less than about 34%, less than about 33%, less than about 32%, less than about 31%, less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, less than about 0.1%, less than about 0.01%, less than about 0.001%, or less than about 0.0001% of the total composition of the cannabis extract.

In some embodiments, a practitioner may choose a cannabis extract “rich” in one cannabinoid acid, and “poor” in another. A non-limiting example includes an “industrial hemp” extract rich in CBDA, and substantially poor in THC/THCA. Per the USDA, “industrial hemp” consists of any part of the plant species Cannabis sativa L., whether growing or not, containing a Δ-9 tetrahydrocannabinol (THC) concentration of no more than three-tenths of one percent (0.3%) on a dry weight basis, measured as total THC by the Colorado Department of Agriculture (CDA) via the post-decarboxylation process.

In such embodiments, an industrial hemp extract rich in CBDA will have a dry-weight percentage of total THC concentration (i.e., both THC and THCA), of less than about 0.3%; including less than about 0.2%, less than about 0.10%, less than about 0.01%, less than about 0.001%, and less than about 0.0001%.

In some embodiments, the cannabis extract possesses substantially no CBCA. In some embodiments, the cannabis extract possesses substantially no CBCVA. In some embodiments, the cannabis extract possesses substantially no CBDA. In some embodiments, the cannabis extract possesses substantially no CBDVA. In some embodiments, the cannabis extract possesses substantially no CBGA. In some embodiments, the cannabis extract possesses substantially no CBGVA. In some embodiments, the cannabis extract possesses substantially no THCA. In some embodiments, the cannabis extract possesses substantially no THCVA. In some embodiments, the cannabis extract possesses substantially no CBDMA. In some embodiments, the cannabis extract possesses substantially no CBDPA. In some embodiments, the cannabis extract possesses substantially no CBEA. In some embodiments, the cannabis extract possesses substantially no CBFA. In some embodiments, the cannabis extract possesses substantially no CBLA. In some embodiments, the cannabis extract possesses substantially no CBNA. In some embodiments, the cannabis extract possesses substantially no CBNDA. In some embodiments, the cannabis extract possesses substantially no CBTA. In some embodiments, the cannabis extract possesses substantially no THCPA.

In some embodiments, the cannabis extract is rich in THCA, and poor in CBCA, CBCVA, CBDA, CBDMA, CBDPA, CBDVA, CBEA, CBFA, CBGA, CBGVA, CBLA, CBNA, CBNDA, CBTA, THCPA, and THCVA.

In some embodiments, the cannabis extract is rich in THCVA, and poor in CBCA, CBCVA, CBDA, CBDMA, CBDPA, CBDVA, CBEA, CBFA, CBGA, CBGVA, CBLA, CBNA, CBNDA, CBTA, THCA, and THCPA.

In some embodiments, the cannabis extract is rich in CBCA, and poor in CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBCVA, and poor in CBCA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDA, and poor in CBCA, CBCVA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDVA, and poor in CBCA, CBCVA, CBDA, CBDMA, CBDPA, CBEA, CBFA, CBGA, CBGVA, CBLA, CBNA, CBNDA, CBTA, THCA, THCPA, and THCVA.

In some embodiments, the cannabis extract is rich in CBGA, and poor in CBCA, CBCVA, CBDA, CBDMA, CBDPA, CBDVA, CBEA, CBFA, CBGVA, CBLA, CBNA, CBNDA, CBTA, THCA, THCPA, and THCVA.

In some embodiments, the cannabis extract is rich in CBGVA, and poor in CBCA, CBCVA, CBDA, CBDMA, CBDPA, CBDVA, CBEA, CBFA, CBGA, CBLA, CBNA, CBNDA, CBTA, THCA, THCPA, and THCVA.

In some embodiments, the cannabis extract is rich in CBDMA, and poor in CBCA, CBCVA, CBDA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDPA, and poor in CBCA, CBCVA, CBDA, CBDMA, CBDVA, CBEA, CBFA, CBGA, CBGVA, CBLA, CBNA, CBNDA, CBTA, THCA, THCPA, and THCVA.

In some embodiments, the cannabis extract is rich in CBEA, and poor in CBCA, CBCVA, CBDA, CBDMA, CBDPA, CBDVA, CBFA, CBGA, CBGVA, CBLA, CBNA, CBNDA, CBTA, THCA, THCPA, and THCVA.

In some embodiments, the cannabis extract is rich in CBFA, and poor in CBCA, CBCVA, CBDA, CBDMA, CBDPA, CBDVA, CBEA, CBGA, CBGVA, CBLA, CBNA, CBNDA, CBTA, THCA, THCPA, and THCVA.

In some embodiments, the cannabis extract is rich in CBLA, and poor in CBCA, CBCVA, CBDA, CBDMA, CBDPA, CBDVA, CBEA, CBFA, CBGA, CBGVA, CBNA, CBNDA, CBTA, THCA, THCPA, and THCVA.

In some embodiments, the cannabis extract is rich in CBNA, and poor in CBCA, CBCVA, CBDA, CBDMA, CBDPA, CBDVA, CBEA, CBFA, CBGA, CBGVA, CBLA, CBNDA, CBTA, THCA, THCPA, and THCVA.

In some embodiments, the cannabis extract is rich in CBNDA, and poor in CBCA, CBCVA, CBDA, CBDMA, CBDPA, CBDVA, CBEA, CBFA, CBGA, CBGVA, CBLA, CBNA, CBTA, THCA, THCPA, and THCVA.

In some embodiments, the cannabis extract is rich in CBTA, and poor in CBCA, CBCVA, CBDA, CBDMA, CBDPA, CBDVA, CBEA, CBFA, CBGA, CBGVA, CBLA, CBNA, CBNDA, THCA, THCPA, and THCVA.

In some embodiments, the cannabis extract is rich in THCPA, and poor in CBCA, CBCVA, CBDA, CBDMA, CBDPA, CBDVA, CBEA, CBFA, CBGA, CBGVA, CBLA, CBNA, CBNDA, CBTA, THCA, and THCVA.

In some embodiments, the cannabis extract is rich in THCA, and has substantially no CBCA, CBCVA, CBDA, CBDMA, CBDPA, CBDVA, CBEA, CBFA, CBGA, CBGVA, CBLA, CBNA, CBNDA, CBTA, THCPA, and THCVA.

In some embodiments, the cannabis extract is rich in THCVA, and has substantially no CBCA, CBCVA, CBDA, CBDMA, CBDPA, CBDVA, CBEA, CBFA, CBGA, CBGVA, CBLA, CBNA, CBNDA, CBTA, THCA, and THCPA.

In some embodiments, the cannabis extract is rich in CBCA, and has substantially no CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBCVA, and has substantially no CBCA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDA, and has substantially no CBCA, CBCVA, CBDMA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDVA, and has substantially no CBCA, CBCVA, CBDA, CBDMA, CBDPA, CBEA, CBFA, CBGA, CBGVA, CBLA, CBNA, CBNDA, CBTA, THCA, THCPA, and THCVA.

In some embodiments, the cannabis extract is rich in CBGA, and has substantially no CBCA, CBCVA, CBDA, CBDMA, CBDPA, CBDVA, CBEA, CBFA, CBGVA, CBLA, CBNA, CBNDA, CBTA, THCA, THCPA, and THCVA.

In some embodiments, the cannabis extract is rich in CBGVA, and has substantially no CBCA, CBCVA, CBDA, CBDMA, CBDPA, CBDVA, CBEA, CBFA, CBGA, CBLA, CBNA, CBNDA, CBTA, THCA, THCPA, and THCVA.

In some embodiments, the cannabis extract is rich in CBDMA, and has substantially no CBCA, CBCVA, CBDA, CBDPA, CBDVA, CBEA, CBFA, CBGA, CBGVA, CBLA, CBNA, CBNDA, CBTA, THCA, THCPA, and THCVA.

In some embodiments, the cannabis extract is rich in CBDPA, and has substantially no CBCA, CBCVA, CBDA, CBDMA, CBDVA, CBEA, CBFA, CBGA, CBGVA, CBLA, CBNA, CBNDA, CBTA, THCA, THCPA, and THCVA.

In some embodiments, the cannabis extract is rich in CBEA, and has substantially no CBCA, CBCVA, CBDA, CBDMA, CBDPA, CBDVA, CBFA, CBGA, CBGVA, CBLA, CBNA, CBNDA, CBTA, THCA, THCPA, and THCVA.

In some embodiments, the cannabis extract is rich in CBFA, and has substantially no CBCA, CBCVA, CBDA, CBDMA, CBDPA, CBDVA, CBEA, CBGA, CBGVA, CBLA, CBNA, CBNDA, CBTA, THCA, THCPA, and THCVA.

In some embodiments, the cannabis extract is rich in CBLA, and has substantially no CBCA, CBCVA, CBDA, CBDMA, CBDPA, CBDVA, CBEA, CBFA, CBGA, CBGVA, CBNA, CBNDA, CBTA, THCA, THCPA, and THCVA.

In some embodiments, the cannabis extract is rich in CBNA, and has substantially no CBCA, CBCVA, CBDA, CBDMA, CBDPA, CBDVA, CBEA, CBFA, CBGA, CBGVA, CBLA, CBNDA, CBTA, THCA, THCPA, and THCVA.

In some embodiments, the cannabis extract is rich in CBNDA, and has substantially no CBCA, CBCVA, CBDA, CBDMA, CBDPA, CBDVA, CBEA, CBFA, CBGA, CBGVA, CBLA, CBNA, CBTA, THCA, THCPA, and THCVA.

In some embodiments, the cannabis extract is rich in CBTA, and has substantially no CBCA, CBCVA, CBDA, CBDMA, CBDPA, CBDVA, CBEA, CBFA, CBGA, CBGVA, CBLA, CBNA, CBNDA, THCA, THCPA, and THCVA.

In some embodiments, the cannabis extract is rich in THCPA, and has substantially no CBCA, CBCVA, CBDA, CBDMA, CBDPA, CBDVA, CBEA, CBFA, CBGA, CBGVA, CBLA, CBNA, CBNDA, CBTA, THCA, and THCVA.

In some embodiments, the cannabis extract is rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any four of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract.

In some embodiments, the cannabis extract is rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in the remaining cannabinoid acid (i.e., the remaining cannabinoid acid comprises less than about 50% of the total composition of the cannabis extract.

In some embodiments, the cannabis extract is rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any four of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acids.

In some embodiments, the cannabis extract is rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possesses substantially none of the remaining cannabinoid acid.

In some embodiments, including in some preferred embodiments, the cannabis extract has a low percentage of neutral cannabinoids compared to cannabinoid acids, for example in a percentage (e.g., by wt/wt) of the total cannabinoid content of less than about 1% neutral cannabinoids of total cannabinoids, or less than about 2%, less than about 3%, less than about 4%, less than about 5%, less than about 6%, less than about 7%, less than about 8%, less than about 9%, less than about 10%, less than about 15%, or less than about 20% neutral cannabinoids of the total cannabinoids of the cannabis extract. In some preferred embodiments, the proportion (e.g., by wt/wt) of neutral cannabinoids to cannabinoid acids in a cannabis extract will be less than about 1:100 neutral cannabinoids:cannabinoid acids, or less than about 1:90, less than about 1:80, less than about 1:70, less than about 1:60, less than about 1:50, less than about 1:40, less than about 1:30, or less than about 1:20 neutral cannabinoids:cannabinoid acids.

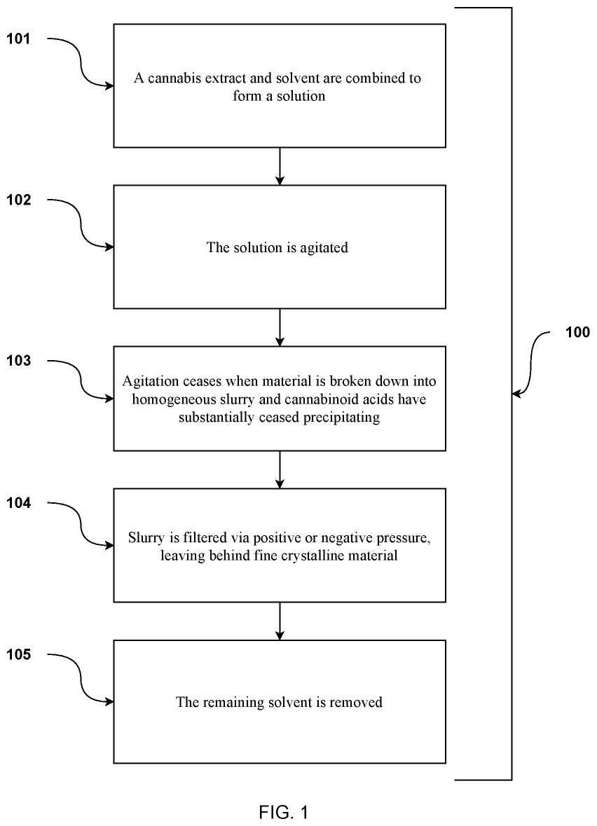

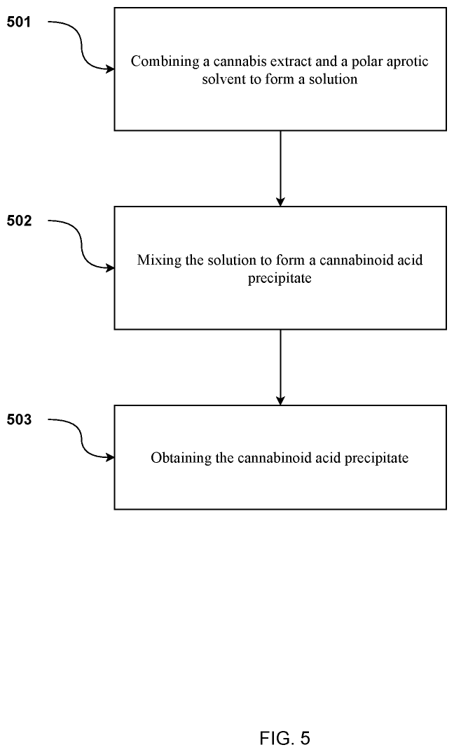

FIG. 1 is a method of facile purification of cannabinoid acids 100 according to an embodiment of the invention. Referring now to FIG. 1, in step 101 a cannabis extract comprising one or more cannabinoid acids and a solvent are combined to form a solution. Reference may also be had to FIG. 5, wherein at step 501 a cannabis extract and a polar aprotic solvent are combined to form a solution.

As would be immediately appreciated by one of skill, the method of combining the extract and solvent are not important, so long as they are combined so as to form a solution. Meaning, in some embodiments, a cannabis extract comprising one or more cannabinoid acids is added to a solvent; while, in other embodiments, a solvent is added to a cannabis extract. Or, in yet other embodiments, an extract and a solvent are simultaneously combined with one another. In addition, as would be apparent to one of skill, regardless of how the extract and solvent are combined (i.e., if the solvent is added to the extract, the extract is added to the solvent, or both are simultaneously combined), the combining step may be completed all at once (meaning, in some embodiments, the solvent and extract are combined in one step); while, in other embodiments, it may be completed in stages-such that the extract and solvent are combined incrementally.

Solvents can generally be classed as nonpolar, polar protic, and polar aprotic. Broadly, a solvent is a substance within which a solute may be dissolved to form a solution—wherein all of the ingredients are relatively uniformly distributed at a molecular level. Solvents are generally liquids, but in some circumstances, may also include solids, gases, or supercritical fluids. Solvents are broadly classified as “polar” or “nonpolar,” wherein the compounds of the former have distinct areas of positive and negative charge (due in part to the unequal sharing of valence electrons during bonding, which is driven by each atom's electronegativity), while the latter do not. In practice, one objective measurement of polarity is a substance's “dielectric constant,” which measures the solvent's ability to cancel the field strength of a charged particle's electric field immersed within the solvent, compared to the field strength of the same in a vacuum (Parekh, Rathod, and Pandit, 2011). In some embodiments, but without being bound by theory, a dielectric constant of 15 or more is considered polar. Other non-limiting examples of polarity metrics include the Grunwald-Winstein mY scale, which measures polarity in terms of solvent influence on the buildup of positively charged solutes during a chemical reaction; the Kosower Z scale, which measures polarity in terms of the influence a solvent has on UV-absorption maxima of a salt (generally pyridinium iodide or the pyridinium zwitterion); and Reichardt's dye, a solvatochromic dye capable of changing color in response to polarity. Functionally, polarity, dipole moment, polarizability, and the ability to hydrogen bond is determinative of what compounds a solvent is able to dissolve into solution.

As it relates to the invention, a “nonpolar” solvent refers to a solvent comprised of at least one “nonpolar compound,” that is, a compound comprised of bonds having “evenly-distributed electrons,” which is reflected in the lack of electrical charge-positive or negative-across the compound (Helmenstine, 2020). Such is the case in compounds composed of atoms having equal, or substantially equal electronegativities. Generally, when a compound possesses two atoms with electronegativities within 0.5 of one another—as measured by the “Pauling” electronegativity scale—the compound possesses non-polar properties (Helmenstine, 2020). As mentioned, because “like dissolves like,” nonpolar compounds are readily dissolved in nonpolar solvents. Broadly, nonpolar solvents include hydrocarbons, i.e., strain-chain molecules or rings comprising at least one carbon atom either saturated with hydrogen (alkanes), including at least one carbon-carbon double bond (alkenes), or having at least one carbon-carbon triple bond (alkynes); or “aromatic” hydrocarbon rings, which contain pi electrons delocalized around them, increasing the stability of the compound (Silberberg, 2004).

Non-limiting examples of nonpolar solvents include methane, ethane, ethene, ethyne, propane, propene, propyne, cyclopropane, propadiene, butane, butene, butyne, cyclobutane, butadiene, pentane, pentene, pentyne, cyclopentane, pentadiene, hexane, hexene, hexyne, cyclohexane, hexadiene, heptane, heptene, heptyne, cycloheptane, heptadiene, octane, octene, octyne, cyclooctane, octadiene, nonane, nonene, nonyne, cyclononane, nonadiene, decane, decene, decyne, cyclodecane, decadiene, undecane, undecene, undecyne, cycloundecane, undecadiene, dodecane, dodecene, dodecyne, cyclododecane, dodecadiene, benzene, methylbenzene (toluene), dimethylbenzene (xylene), ethylbenzene; and polynuclear aromatic hydrocarbons, including but not limited to naphthalene, acenaphthylene, acenaphthene, fluorene, anthracene, phenanthrene, fluoranthene, benzo[b]fluoranthene, benzo[f]fluoranthene, benzo[k]fluoranthene, benzo[a]anthracene, chrysene, pyrene, benzo[a]pyrene, dibenzo[a,l]pyrene, dibenzo[a,e]pyrene, dibenz[a,h]anthracene, anthanthrene, benzo[g,h,i]perylene, and indeno[1,2,3-cd]pyrene.

Polar solvents generally have large dipole moments, and the capability to dissolve ions and other polar compounds. Broadly, “dipole moments” occur when there is a separation of charge between two ions in an ionic bond, or two atoms in a covalent bond (Blaber, 2020). Naturally, this occurs when atoms of substantially different electronegativities form a bond with one another. Thus, the “dipole moment” can be said to be a measure of the polarity of the molecule, given a larger difference of electronegativities leads to an increase in the polarity of the resultant molecule. Said another way, the unequal sharing of electrons-characteristic of polar compounds, as mentioned-itself creates a dipole moment. More specifically, when two electrical charges of opposing sign and equal magnitude are separated by a distance, an electric dipole is established (Blaber, 2020). The specific size of the dipole moment, as measured in Debye units-which are equal to the distance between the charges multiplied by the charge—is expressed by the equation

$$\vec{μ} = {\sum }_{i} {q}_{i} {\vec{r}}_{i} ,$$

wherein β is the dipole moment vector, q, is the magnitude of the i^{th} charge, and r, is the vector representing the position of i^{th} charge. The dipole moment itself acts in the direction of the vector quantity, from the positive portion of the molecule, i.e., the atom having a lower electronegativity, to the negative portion of the molecule, i.e., to the atom having a higher electronegativity (Blaber, 2020). Numerically, dipole moments can be calculated for individual bonds, wherein the range of dipole moments is between about 0 D to about 11 D, and can be calculated for the molecule as a whole by adding all the individual dipoles of the individual bonds as their vector.

In some embodiments, a polar solvent useful in the invention has a molecular dipole moment of between about 0.1 and about 11 D, including about 0.1 D, about 0.2 D, about 0.3 D, about 0.4 D, about 0.5 D, about 0.6 D, about 0.7 D, about 0.8 D, about 0.9 D, about 1 D, about 1.1 D, about 1.2 D, about 1.3 D, about 1.4 D, about 1.5 D, about 1.6 D, about 1.7 D, about 1.8 D, about 1.9 D, about 2 D, about 2.1 D, about 2.2 D, about 2.3 D, about 2.4 D, about 2.5 D, about 2.6 D, about 2.7 D, about 2.8 D, about 2.9 D, about 3 D, about 3.1 D, about 3.2 D, about 3.3 D, about 3.4 D, about 3.5 D, about 3.6 D, about 3.7 D, about 3.8 D, about 3.9 D, about 4 D, about 4.1 D, about 4.2 D, about 4.3 D, about 4.4 D, about 4.5 D, about 4.6 D, about 4.7 D, about 4.8 D, about 4.9 D, about 5 D, about 5.1 D, about 5.2 D, about 5.3 D, about 5.4 D, about 5.5 D, about 5.6 D, about 5.7 D, about 5.8 D, about 5.9 D, about 6 D, about 6.1 D, about 6.2 D, about 6.3 D, about 6.4 D, about 6.5 D, about 6.6 D, about 6.7 D, about 6.8 D, about 6.9 D, about 7 D, about 7.1 D, about 7.2 D, about 7.3 D, about 7.4 D, about 7.5 D, about 7.6 D, about 7.7 D, about 7.8 D, about 7.9 D, about 8 D, about 8.1 D, about 8.2 D, about 8.3 D, about 8.4 D, about 8.5 D, about 8.6 D, about 8.7 D, about 8.8 D, about 8.9 D, about 9 D, about 9.1 D, about 9.2 D, about 9.3 D, about 9.4 D, about 9.5 D, about 9.6 D, about 9.7 D, about 9.8 D, about 9.9 D, about 10 D, about 10.1 D, about 10.2 D, about 10.3 D, about 10.4 D, about 10.5 D, about 10.6 D, about 10.7 D, about 10.8 D, about 10.9 D, about 11 D, or values in between, as would be apparent to one of skill.

In some embodiments, a polar solvent useful in the invention is composed of a plurality of bonds (e.g., at least one, at least two, at least three, at least four, at least five, at least six, at least seven, at least eight, at least nine, at least ten, at least eleven, at least twelve, at least thirteen, at least fourteen at least fifteen, at least sixteen, at least seventeen, at least eighteen, at least nineteen, at least twenty, at least twenty five, at least thirty, and values in between, as would be apparent to one of skill), wherein each of the plurality of bonds has a dipole moment of between about 0 and about 11 D, including about 0 D, about 0.1 D, about 0.2 D, about 0.3 D, about 0.4 D, about 0.5 D, about 0.6 D, about 0.7 D, about 0.8 D, about 0.9 D, about 1 D, about 1.1 D, about 1.2 D, about 1.3 D, about 1.4 D, about 1.5 D, about 1.6 D, about 1.7 D, about 1.8 D, about 1.9 D, about 2 D, about 2.1 D, about 2.2 D, about 2.3 D, about 2.4 D, about 2.5 D, about 2.6 D, about 2.7 D, about 2.8 D, about 2.9 D, about 3 D, about 3.1 D, about 3.2 D, about 3.3 D, about 3.4 D, about 3.5 D, about 3.6 D, about 3.7 D, about 3.8 D, about 3.9 D, about 4 D, about 4.1 D, about 4.2 D, about 4.3 D, about 4.4 D, about 4.5 D, about 4.6 D, about 4.7 D, about 4.8 D, about 4.9 D, about 5 D, about 5.1 D, about 5.2 D, about 5.3 D, about 5.4 D, about 5.5 D, about 5.6 D, about 5.7 D, about 5.8 D, about 5.9 D, about 6 D, about 6.1 D, about 6.2 D, about 6.3 D, about 6.4 D, about 6.5 D, about 6.6 D, about 6.7 D, about 6.8 D, about 6.9 D, about 7 D, about 7.1 D, about 7.2 D, about 7.3 D, about 7.4 D, about 7.5 D, about 7.6 D, about 7.7 D, about 7.8 D, about 7.9 D, about 8 D, about 8.1 D, about 8.2 D, about 8.3 D, about 8.4 D, about 8.5 D, about 8.6 D, about 8.7 D, about 8.8 D, about 8.9 D, about 9 D, about 9.1 D, about 9.2 D, about 9.3 D, about 9.4 D, about 9.5 D, about 9.6 D, about 9.7 D, about 9.8 D, about 9.9 D, about 10 D, about 10.1 D, about 10.2 D, about 10.3 D, about 10.4 D, about 10.5 D, about 10.6 D, about 10.7 D, about 10.8 D, about 10.9 D, about 11 D, or values in between, as would be apparent to one of skill; and has at least one bond of at least about 0.1 D, including about 0.2 D, about 0.3 D, about 0.4 D, about 0.5 D, about 0.6 D, about 0.7 D, about 0.8 D, about 0.9 D, about 1 D, about 1.1 D, about 1.2 D, about 1.3 D, about 1.4 D, about 1.5 D, about 1.6 D, about 1.7 D, about 1.8 D, about 1.9 D, about 2 D, about 2.1 D, about 2.2 D, about 2.3 D, about 2.4 D, about 2.5 D, about 2.6 D, about 2.7 D, about 2.8 D, about 2.9 D, about 3 D, about 3.1 D, about 3.2 D, about 3.3 D, about 3.4 D, about 3.5 D, about 3.6 D, about 3.7 D, about 3.8 D, about 3.9 D, about 4 D, about 4.1 D, about 4.2 D, about 4.3 D, about 4.4 D, about 4.5 D, about 4.6 D, about 4.7 D, about 4.8 D, about 4.9 D, about 5 D, about 5.1 D, about 5.2 D, about 5.3 D, about 5.4 D, about 5.5 D, about 5.6 D, about 5.7 D, about 5.8 D, about 5.9 D, about 6 D, about 6.1 D, about 6.2 D, about 6.3 D, about 6.4 D, about 6.5 D, about 6.6 D, about 6.7 D, about 6.8 D, about 6.9 D, about 7 D, about 7.1 D, about 7.2 D, about 7.3 D, about 7.4 D, about 7.5 D, about 7.6 D, about 7.7 D, about 7.8 D, about 7.9 D, about 8 D, about 8.1 D, about 8.2 D, about 8.3 D, about 8.4 D, about 8.5 D, about 8.6 D, about 8.7 D, about 8.8 D, about 8.9 D, about 9 D, about 9.1 D, about 9.2 D, about 9.3 D, about 9.4 D, about 9.5 D, about 9.6 D, about 9.7 D, about 9.8 D, about 9.9 D, about 10 D, about 10.1 D, about 10.2 D, about 10.3 D, about 10.4 D, about 10.5 D, about 10.6 D, about 10.7 D, about 10.8 D, about 10.9 D, about 11 D, or values in between, as would be apparent to one of skill.

As mentioned, the dipole moment of a given bond or molecule is primarily due to partial positive and negative charges spanning the compound resulting from the unequal “sharing” of electrons between atoms chemically bonded to one another. The ability of an atom of a given element to attract shared electrons when forming a chemical bond is termed its “electronegativity,” which is largely impacted by both its atomic number and the distance at which its valence electrons (the electrons that participate in bonding) reside from the nucleus (IUPAC, 1997). As electronegativity increases, so does the atom's ability to attract electrons, and so too does the polarity of the bond that forms. Because “like dissolves like,” polar compounds are readily dissolved in polar solvents.

In some embodiments, a polar solvent will have a dielectric constant greater than 15. In some embodiments, the dielectric constant that is at least about 15, including at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, and at least about 100.

In some embodiments, a polar solvent is a polar protic solvent, which has any of O—H bonds, N—H bonds, or a combination thereof, and wherein the solvent has the ability to solvate solutes (generally conjugate bases) via hydrogen bonding (Ashenhurst, 2020). Common polar protic solvents include, but are not limited to, alcohols, such as methanol, ethanol, 1-propanol, 2-propanol, 1-butanol, 2-butanol, 2-methyl-1-propanol, 2-methyl-2-propanol, 1-pentanol, 3-methyl-1-butanol, 2,2-dimethyl-1-propanol, cyclopentanol, 1-hexanol, cyclohexanol, 1-heptanol, 1-octanol, 1-nonanol, 1-decanol, 2-propen-1-ol, phenylmethanol, diphenylmethanol, and triphenylmethanol; ammonia, acetic acid, ethylene glycol, pyridine, diethylene glycol, cyclohexanone, chloroform, water, etc.

In some embodiments, a polar solvent is an aprotic solvent. Structurally, “aprotic” solvents lack an acidic protein and hydroxyl/amine groups. Functionally, and without being bound by theory, aprotic solvents do not serve as proton donors in hydrogen bonding, though they can be proton acceptors (Ashenhurst, 2020). Aprotic solvents generally have large dipole moments, and solvate charged species such as anions used as nucleophiles. However, the lack of hydrogen bonding in aprotic solvents leads to such nucleophiles being relatively “free” in solution, making them more reactive (Ashenhurst, 2020). Common polar aprotic solvents include, but are not limited to, acetone, acetonitrile, benzonitrile, butyronitrile, decanenitrile, dichloromethane, dimethylformamide, dimethylpropyleneurea, dimethylsulfoxide, ethyl acetate, hexamethylphosphoric triamide, isobutyronitrile, malononitrile, nitromethane, pivalonitrile, propionitrile, propylene carbonate, succinonitrile, and tetrahydrofuran.

In some embodiments, the solvent will not be heptane. In some embodiments, the solvent will not be any of hexane, pentane, isopropyl alcohol, ethanol, or hydrocarbons. In some embodiments, the solvent will not be any of hexane, pentane, isopropyl alcohol, ethanol, hydrocarbons, or mixtures thereof. In some embodiments, the solvent will not be acetone. In some embodiments, the solvent will not be any of n-propanol, 1-butanol, acetone, or acetonitrile. In some embodiments, the solvent will not be any of n-propanol, 1-butanol, acetone, acetonitrile, or mixtures thereof. In some embodiments, the solvent will not be any of hexane, pentane, isopropyl alcohol, ethanol, hydrocarbons, n-propanol, DMA, heptane, 1-butanol, ethyl acetate, acetone, or acetonitrile. In some embodiments, the solvent will not be any of hexane, pentane, isopropyl alcohol, ethanol, hydrocarbons, n-propanol, DMA, heptane, 1-butanol, ethyl acetate, acetone, acetonitrile, or mixtures thereof. In some embodiments, the solvent will not be acetonitrile. In some embodiments, the solvent will be an aprotic solvent, but will not be acetonitrile. In some embodiments, the solvent will be a polar aprotic solvent, but will not be acetonitrile. In some embodiments, the solvent will be a nitrile aprotic solvent, but will not be acetonitrile. In some embodiments, the solvent will be a polar nitrile aprotic solvent, but will not be acetonitrile. In some embodiments, the aprotic solvent will not be ethyl acetate. In some embodiments, the aprotic solvent will not be tetrahydrofuran. In some embodiments, the aprotic solvent will not be dimethylformamide. In some embodiments, the aprotic solvent will not be dimethylacetamide. In some embodiments, the aprotic solvent will not be any of ethyl acetate, tetrahydrofuran, dimethylformamide, or dimethylacetamide. In some embodiments, the aprotic solvent will not be dimethylacetamide. In some embodiments, the aprotic solvent will not be any of ethyl acetate, tetrahydrofuran, dimethylformamide, dimethylacetamide, or mixtures thereof.

In some embodiments, acetonitrile is used as the polar aprotic solvent of the invention. “Acetonitrile,” also known as “cyanomethane,” “ethyl nitrile,” “ethanenitrile,” “methanecarbonitrile,” and “methyl cyanide,” refers to a compound with the formula H₃C—C≡N that is a substantially colorless liquid with an ether-like odor. Chemically, acetonitrile is highly volatile, can react with water, steam, or acids to produce flammable vapors, and is used in the production of pharmaceuticals, perfumes, rubber products, pesticides, acrylic nail removers, and batteries. Acetonitrile has a free electron pair at the nitrogen atom, which can form many transition metal nitrile complexes. As a weakly-basic compound, acetonitrile is also an easily displaceable ligand (Jurgen-Hinrich and Fuhrhop, 2003). Acetonitrile may be produced or purchased, and is available from sources like Chemsavers, Inc., Fisher Scientific, ACB SciTech, ChemDirect, Lab Alley, Sigma-Aldrich, American Chemical Suppliers, and Science Company; while it is produced largely as a byproduct from the manufacture of acrylonitrile via the dehydration of acetamide or by the hydrogenation of mixtures of carbon monoxide and ammonia (Olive and Olive, 1979).

Other polar aprotic solvents useful in the practice of the invention broadly include nitrile aprotic solvents, such as generally organic compounds having a dielectric constant greater than about 15 that also contain a —C≡N functional group, and no N—H or O—H bonds. Non-limiting examples of nitrile aprotic solvents, other than acetonitrile, include acrylonitrile, benzonitrile, butyronitrile, decanenitrile, pivalonitrile (trimethylacetonitrile), propionitrile, isobutyronitrile, malononitrile, succinonitrile, cyanoacetylene, dicyanoacetylene, and such others as disclosed herein and generally known to those in the art (see, e.g., U.S. Pub. No. 2004/0024238A1). In some embodiments, the nitrile aprotic solvent is any of acetonitrile, butyronitrile, isobutyronitrile, propionitrile, malononitrile, and succinonitrile. In some embodiments, a solvent used in the practice of the methods of the invention will be a mixture of one or more solvents, such as one or more polar aprotic solvents and/or one or more nitrile aprotic solvents, and including combinations thereof.

In some embodiments, a polar aprotic nitrile solvent of the invention is characterized by R—CN wherein R is C1—C9 alkyl, —CH═CH₂, -Phenyl, —CH₂CN, —CH₂CH₂CN, —CH₂CH₂CH₂CN, —CN, —C—CH, and —C—C—CN. The term “alkyl” refers to straight chain or branched alkyl substituent.

In some embodiments, a polar aprotic nitrile solvent of the invention is characterized by R—CN wherein R represents a hydrocarbon group having from 1 to 9 carbon atoms which optionally has a substituent, or a nitrile group.

In some embodiments, a polar aprotic nitrile solvent of the invention is characterized by R—CN wherein R represents —(CH₂)˜CN or —(CH₂)˜CH₃, wherein n is from 1 to 9.

In some embodiments, R is CT-C5 alkyl, —CH═CH₂, —CN, and —C≡CH. The term alkyl refers to straight chain or branched alkyl substituent.

In some embodiments, R is CT-C2 alkyl, —CH═CH₂, —CN, and —C≡CH.

In some embodiments, the polar aprotic nitrile solvent is H₃C—C≡N.

In some embodiments, solvents useful in the practice of the invention include organic solvents containing a cyano—(C≡N) bonded to another carbon atom, such as acetonitrile, propionitrile, C₂₋₆ nitriles, or mixtures thereof.

In some embodiments, solvents useful in the practice of the invention include polar aprotic solvents having a dielectric constant greater than 15, for example any of the amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO); as well as mixtures thereof. In some embodiments, the solvent will be a polar aprotic solvent having a dielectric constant greater than 15, and will not be any of THF, DMF, or DMA, or mixtures thereof.

In some embodiments, solvents useful in the practice of the invention include polar aprotic nitrile solvents having a melting point below about 40° C., and a boiling point below about 180° C. One reason for this, but without being bound by theory, is due to processing feasibility in certain embodiments herein, as will be appreciated by those in the art. If a solution must be warmed above about 40° C. to remain a liquid, the processing temperature may be too warm to reliably yield a cannabis acid precipitate within other normal operating parameters. Likewise, if a solution boils above 180° C., it may be difficult to recover the solvent for reuse, or to fully purge the solvent out of the remaining extract that is rich in flavor compounds, including but not limited to terpenes, esters, aldehydes, and ketones. Additionally, in distilling a high boiling point solvent, there may not be a clean separation between solvent and flavor compounds such that a significantly greater amount of flavor compounds will be distilled over with the solvent. Nevertheless, it will be within the practice of ordinary skill and will not require significant experimentation to determine the processing feasibility and select processing parameters for all solvents disclosed herein. In some preferred embodiments however, a solvent useful in the practice of the invention is a polar aprotic nitrile solvent having a melting point of about 39.9° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 10° C., about 9° C., about 8° C., about 7° C., about 6° C., about 5° C., about 4° C., about 3° C., about 2° C., about 1° C., about 0° C., about −1° C., about −2° C., about −3° C., about −4° C., about −5° C., about −6° C., about −7° C., about −8° C., about −9° C., about −10° C., about −11° C., about −12° C., about −13° C., about −14° C., about −15° C., about −16° C., about −17° C., about −18° C., about −19° C., about −20° C., about −21° C., about −22° C., about −23° C., about −24° C., about −25° C., about −26° C., about −27° C., about −28° C., about −29° C., about −30° C., about −31° C., about −32° C., about −33° C., about −34° C., about −35° C., about −36° C., about −37° C., about −38° C., about −39° C., about −40° C., about −41° C., about −42° C., about −43° C., about −44° C., about −45° C., about −46° C., about −47° C., about −48° C., about −49° C., about −50° C., about −51° C., about −52° C., about −53° C., about −54° C., about −55° C., about −56° C., about −57° C., about −58° C., about −59° C., about −60° C., and values in between, as would be appreciated by once of skill. In some embodiments, a solvent useful in the practice of the invention is a polar aprotic nitrile solvent having a melting point below about −60° C., including about −75° C., about −90° C., about −100° C. and, in some embodiments, below about −100° C.

In some embodiments, solvents useful in the practice of the invention include polar aprotic nitrile solvents having a melting point below about 10° C., including about 10° C., about 9° C., about 8° C., about 7° C., about 6° C., about 5° C., about 4° C., about 3° C., about 2° C., about 1° C., about 0° C., about −1° C., about −2° C., about −3° C., about −4° C., about −5° C., about −6° C., about −7° C., about −8° C., about −9° C., about −10° C., about −11° C., about −12° C., about −13° C., about −14° C., about −15° C., about −16° C., about −17° C., about −18° C., about −19° C., about −20° C., about −21° C., about −22° C., about −23° C., about −24° C., about −25° C., about −26° C., about −27° C., about −28° C., about −29° C., about −30° C., about −31° C., about −32° C., about −33° C., about −34° C., about −35° C., about −36° C., about −37° C., about −38° C., about −39° C., about −40° C., about −41° C., about −42° C., about −43° C., about −44° C., about −45° C., about −46° C., about −47° C., about −48° C., about −49° C., about −50° C., about −51° C., about −52° C., about −53° C., about −54° C., about −55° C., about −56° C., about −57° C., about −58° C., about −59° C., about −60° C., and values in between, as appreciated by once of skill.

In some embodiments, solvents useful in the practice of the invention include polar aprotic nitrile solvents having a boiling point of about 179.9° C., about 179° C., about 178° C., about 177° C., about 176° C., about 175° C., about 174° C., about 173° C., about 172° C., about 171° C., about 170° C., about 169° C., about 168° C., about 167° C., about 166° C., about 165° C., about 164° C., about 163° C., about 162° C., about 161° C., about 160° C., about 159° C., about 158° C., about 157° C., about 156° C., about 155° C., about 154° C., about 153° C., about 152° C., about 151° C., about 150° C., about 149° C., about 148° C., about 147° C., about 146° C., about 145° C., about 144° C., about 143° C., about 142° C., about 141° C., about 140° C., about 139° C., about 138° C., about 137° C., about 136° C., about 135° C., about 134° C., about 133° C., about 132° C., about 131° C., about 130° C., about 129° C., about 128° C., about 127° C., about 126° C., about 125° C., about 124° C., about 123° C., about 122° C., about 121° C., about 120° C., about 119° C., about 118° C., about 117° C., about 116° C., about 115° C., about 114° C., about 113° C., about 112° C., about 111° C., about 110° C., about 109° C., about 108° C., about 107° C., about 106° C., about 105° C., about 104° C., about 103° C., about 102° C., about 101° C., about 100° C., about 99° C., about 98° C., about 97° C., about 96° C., about 95° C., about 94° C., about 93° C., about 92° C., about 91° C., about 90° C., about 89° C., about 88° C., about 87° C., about 86° C., about 85° C., about 84° C., about 83° C., about 82° C., about 81° C., about 80° C., about 79° C., about 78° C., about 77° C., about 76° C., about 75° C., about 74° C., about 73° C., about 72° C., about 71° C., about 70° C., about 69° C., about 68° C., about 67° C., about 66° C., about 65° C., about 64° C., about 63° C., about 62° C., about 61° C., about 60° C., about 59° C., about 58° C., about 57° C., about 56° C., about 55° C., about 54° C., about 53° C., about 52° C., about 51° C., about 50° C., about 49° C., about 48° C., about 47° C., about 46° C., about 45° C., about 44° C., about 43° C., about 42° C., about 41° C., about 40° C., and values in between, as would be apparent to one of skill.

In some embodiments, solvents useful in the practice of the invention include polar aprotic nitrile solvents having a boiling point below about 100° C., including about 100° C., about 99° C., about 98° C., about 97° C., about 96° C., about 95° C., about 94° C., about 93° C., about 92° C., about 91° C., about 90° C., about 89° C., about 88° C., about 87° C., about 86° C., about 85° C., about 84° C., about 83° C., about 82° C., about 81° C., about 80° C., about 79° C., about 78° C., about 77° C., about 76° C., about 75° C., about 74° C., about 73° C., about 72° C., about 71° C., about 70° C., about 69° C., about 68° C., about 67° C., about 66° C., about 65° C., about 64° C., about 63° C., about 62° C., about 61° C., about 60° C., about 59° C., about 58° C., about 57° C., about 56° C., about 55° C., about 54° C., about 53° C., about 52° C., about 51° C., about 50° C., about 49° C., about 48° C., about 47° C., about 46° C., about 45° C., about 44° C., about 43° C., about 42° C., about 41° C., about 40° C., and values in between, as would be apparent to one of skill.

In some embodiments, the polar aprotic solvent will not be any of N,N-dimethylacetamide (DMA), N,N-dimethylformamide (DMF), or tetrahydrofuran (THF), or mixtures thereof. In some embodiments, the polar aprotic solvent will not be a mixture of N,N-dimethylacetamide (DMA), N,N-dimethylformamide (DMF), and tetrahydrofuran (THF). In some embodiments, the polar aprotic solvent will not be a mixture of N,N-dimethylacetamide (DMA) and tetrahydrofuran (THF). In some embodiments, the polar aprotic solvent will not be a mixture of N,N-dimethylformamide (DMF) and tetrahydrofuran (THF). In some embodiments, the polar aprotic solvent will not be N,N-dimethylacetamide (DMA). In some embodiments, the polar aprotic solvent will not be N,N-dimethylformamide (DMF). In some embodiments, the polar aprotic solvent will not be tetrahydrofuran (THF).

Other solvents useful in the practice of some embodiments of the invention include polar solvents, non-limiting examples of which include alcohols, such as methanol, ethanol, 1-propanol, 2-propanol, 1-butanol, 2-butanol, 2-methyl-1-propanol, 2-methyl-2-propanol, 1-pentanol, 3-methyl-1-butanol, diethylene glycol, 2,2-dimethyl-1-propanol, ethylene glycol, cyclopentanol, 1-hexanol, cyclohexanol, 1-heptanol, 1-octanol, 1-nonanol, 1-decanol, 2-propen-1-ol, phenylmethanol, diphenylmethanol, and triphenylmethanol; ammonia, acetic acid, pyridine, diethylene glycol, cyclohexanone, and chloroform; and non-nitrile polar aprotic solvents, including acetone, dichloromethane, dimethylformamide, dimethylpropyleneurea, dimethylsulfoxide, ethyl acetate, hexamethylphosphoric triamide, nitromethane, propylene carbonate, and tetrahydrofuran.

Next, in step 102, the mixture is agitated, for example to break apart the chunks of crystalline grains in the extract and/or to help dissolve any amorphous material that may be trapped between crystalline grains. Reference may also be had to FIG. 5, wherein at step 502 the cannabis extract and the polar aprotic solvent which were combined in step 501 to form a solution are now mixed, to form a cannabinoid acid precipitate. In some embodiments, agitation is performed with a magnetic stir bar or high-shear blade. In some embodiments, agitation is performed manually, through the use of any substantially solid material capable of breaking apart any chunks of crystalline grains in the extract, a non-limiting example being a spatula or other stirrer. As the agitation is performed the extract is broken down into finer grains of crystalline cannabinoid acids (the cannabinoid acid precipitate) while other unwanted compounds are dissolved. Additionally, cannabinoid acids which were previously dissolved in the amorphous material begin to crystallize out of solution (precipitate). In some embodiments, the mixture is agitated for a predetermined time, the time selected by one of skill based on the characteristics of the (amount, purity, etc.), and/or the qualities of the desired end product. In some embodiments, agitation is continued until all visible chunks of extract material have been broken down into a homogeneous slurry. In some embodiments, agitation is stopped when all visible chunks of extract material have been broken down into a homogeneous slurry. In some embodiments, the mixture is agitated for between about 10 minutes to about 36 hours, including about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 90 minutes, about 120 minutes, about 150 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, and times in between as would be immediately apparent to one of skill. Note, however, while a range of between about 10 minutes to about 36 hours is typical, as would be appreciated by those of skill, processing times will be dependent on the extract purity, stirring speed, blade shape, and temperature of the slurry. A “predetermined time” will include one that can be determined within the practice of ordinary skill, based on the teachings herein and the general knowledge in the art, and without undue experimentation (e.g., by performing the process a relatively small number of times to optimize the parameters in light of operating and design considerations).

In some embodiments, wherein a stirring speed is measured in revolutions per minute (rpm), the stirring speed is between about 300 rpm to about 1,500 rpm. In some embodiments, wherein stirring speed is measured in rpm, the stirring speed is about 300 rpm, about 350 rpm, about 400 rpm, about 450 rpm, about 500 rpm, about 550 rpm, about 600 rpm, about 650 rpm, about 700 rpm, about 750 rpm, about 800 rpm, about 850 rpm, about 900 rpm, about 950 rpm, about 1,000 rpm, about 1,050 rpm, about 1,100 rpm, about 1,150 rpm, about 1,200 rpm, about 1,250 rpm, about 1,300 rpm, about 1,350 rpm, about 1,400 rpm, about 1,450 rpm, or about 1,500 rpm. In some embodiments, the stirring speed is below about 300 rpm. In some embodiments, the stirring speed is above about 1500 rpm.

In some embodiments, the mixture containing the cannabis extract and solvent is added to a vessel, non-limiting examples of which include vats, kettles, and caldrons constructed of a substantially solid material, including but not limited to stainless steel, copper, and glass. Herein, “vessel” or “processing vessel” refers to a substantially solid structure having a top, a bottom, a means of accessing the interior of the vessel, including but not limited to a hatch or lid, an internal area, and at least one side operably connected to the top and to the bottom so as to form a substantially sealed container that prevents the leakage of the contents therein, capable of holding a plurality of substances, including but not limited to solids, such as plant matter; liquids, such as liquid extracts, solvents, as defined herein; a homogeneous or substantially homogeneous slurry, as defined herein; and gasses, such as those produced as a result of the chemical reactions of the invention.

In some embodiments, a vessel may be cylindrical in shape, wherein the at least one side is a single side operably connected (via means known to those of skill) to the top and the bottom; in some embodiments, the vessel may be spherical in shape, wherein the at least one side is a single side operably connected (via means known to those of skill) to the top and the bottom; in some embodiments, the vessel may be a cube, wherein the at least one side includes four sides of equal length and width that are operably connected (via means known to those of skill) to themselves, and to the top and the bottom. In some embodiments, the vessel may be a rectangular prism, wherein the at least one side includes four sides, each having a longer side and a shorter side, that are operably connected (via means known to those of skill) to themselves, and to the top and the bottom. In some embodiments, the vessel may be a triangular prism, wherein the top and the bottom are both triangular (i.e., comprised of three sides having interior angles adding up to 180°), and wherein the at least one side includes three sides of the same size and shape that are operably connected (via means known to those of skill) to themselves, and to the top and the bottom. While cylindrical, spherical, cuboidal, and rectangular/triangular prisms are shapes that are useful for the vessel of the invention herein, such shapes are merely exemplary and not intended to limit the scope of the invention; any shape known to one of skill as being useful to carry out the process of the invention may be utilized for the process of the invention as disclosed herein.

In some embodiments, the vessel is placed in a water bath maintained from between about 40° C. to about 80° C., including about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., and values in between, as would be apparent to one of skill.

In some embodiments, wherein the vessel is placed in a heated water bath to lower the slurry's viscosity and facilitate stirring, once the slurry is sufficiently stirred, the vessel is removed from the water bath and allowed to cool for between about 20 minutes to about 40 minutes, including about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, and values in between, as apparent to one of skill.

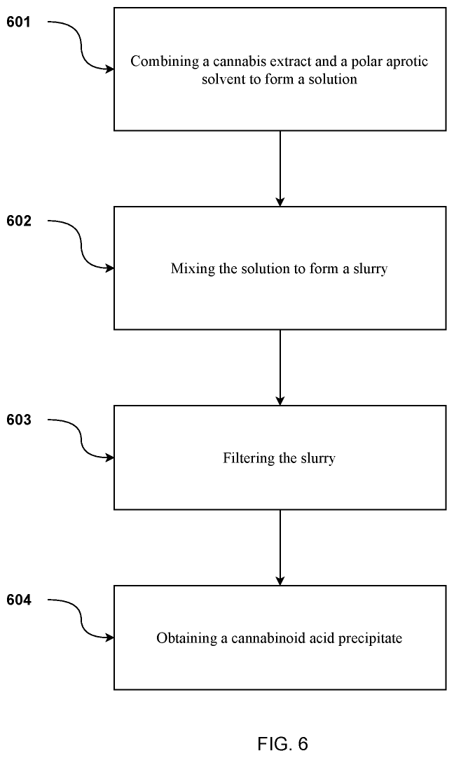

In some embodiments, the agitation of step 102 creates a slurry comprising the solvent and extract material. Reference may also be had to FIG. 6, wherein at step 602 the cannabis extract and the polar aprotic solvent that were combined to form a solution in step 601 are mixed to form a slurry. A “slurry,” or a “homogeneous slurry,” is a homogeneous mixture (and in some embodiments, a “substantially homogeneous slurry,” and therefore a substantially homogeneous mixture) comprising a cannabis extract, as described herein, and a solvent, such as also described herein. “Substantially homogeneous” refers to a mixture having a substantially uniform composition. Broadly, a “slurry” refers to a mixture of solids denser than water suspended in a liquid. Thus, a substantially homogeneous slurry in some embodiments of the invention refers to the cannabis extract sufficiently dissolved within the solvent to the extent that the mixture appears to be (to an observer of ordinary skill), and behaves as, a homogeneous liquid. It will be readily appreciated that although in some embodiments the solids (e.g., the cannabinoid acid precipitate) will remain suspended and be substantially uniformly dispersed, solids may nonetheless begin to settle toward the bottom under the force of gravity, and begin to stratify and over time become less homogeneous and begin to become “pseudo-homogeneous” (e.g., a mixture in which the solids are in suspension, but where the concentration is greater towards the bottom); and, over longer periods of time, tend to become more heterogeneous (e.g., a mixture in which the solids are not uniformly distributed and tend to be more concentrated in the bottom of the containment vessel). Such slurry characteristics and the terminology used to describe them will be readily appreciated and will be known to those of ordinary skill, and understood depending on and in view of the particular context. A “substantially homogeneous” slurry, for example, may appear visibly homogenous to those of skill, but be at least partially pseudo-homogeneous or non-homogeneous when analyzed through precise quantitative means. Accordingly, “substantially homogeneous,” herein, will refer to mixtures that are quantitatively homogeneous as well as those that are qualitatively homogeneous to observers of skill in the art, e.g., that appear homogeneous to a practitioner or operator of the processes of the invention, who is one of skill in the art, such as through direct visual inspection or means.

In some embodiments, a solvent (which may be a polar aprotic solvent, including an organic nitrile solvent such as acetonitrile) is added to the cannabis extract at a rate of between about 200 mL to about 1,000 mL of solvent per about 1,000 g of extract. In some embodiments, said solvent (hereinafter in this paragraph now “acetonitrile,” but including any other solvent of the invention) is added to the cannabis extract at a rate of about 200 mL of solvent per about 1,000 g of extract. In some embodiments, acetonitrile is added to the cannabis extract at a rate of about 210 mL of solvent per about 1,000 g of extract. In some embodiments, acetonitrile is added to the cannabis extract at a rate of about 220 mL of solvent per about 1,000 g of extract. In some embodiments, acetonitrile is added to the cannabis extract at a rate of about 230 mL of solvent per about 1,000 g of extract. In some embodiments, acetonitrile is added to the cannabis extract at a rate of about 240 mL of solvent per about 1,000 g of extract. In some embodiments, acetonitrile is added to the cannabis extract at a rate of about 250 mL of solvent per about 1,000 g of extract. In some embodiments, acetonitrile is added to the cannabis extract at a rate of about 260 mL of solvent per about 1,000 g of extract. In some embodiments, acetonitrile is added to the cannabis extract at a rate of about 270 mL of solvent per about 1,000 g of extract. In some embodiments, acetonitrile is added to the cannabis extract at a rate of about 280 mL of solvent per about 1,000 g of extract. In some embodiments, acetonitrile is added to the cannabis extract at a rate of about 290 mL of solvent per about 1,000 g of extract. In some embodiments, acetonitrile is added to the cannabis extract at a rate of about 300 mL of solvent per about 1,000 g of extract. In some embodiments, acetonitrile is added to the cannabis extract at a rate of about 310 mL of solvent per about 1,000 g of extract. In some embodiments, acetonitrile is added to the cannabis extract at a rate of about 320 mL of solvent per about 1,000 g of extract. In some embodiments, acetonitrile is added to the cannabis extract at a rate of about 330 mL of solvent per about 1,000 g of extract. In some embodiments, acetonitrile is added to the cannabis extract at a rate of about 340 mL of solvent per about 1,000 g of extract. In some embodiments, acetonitrile is added to the cannabis extract at a rate of about 350 mL of solvent per about 1,000 g of extract. In some embodiments, acetonitrile is added to the cannabis extract at a rate of about 360 mL of solvent per about 1,000 g of extract. In some embodiments, acetonitrile is added to the cannabis extract at a rate of about 370 mL of solvent per about 1,000 g of extract. In some embodiments, acetonitrile is added to the cannabis extract at a rate of about 380 mL of solvent per about 1,000 g of extract. In some embodiments, acetonitrile is added to the cannabis extract at a rate of about 390 mL of solvent per about 1,000 g of extract. In some embodiments, acetonitrile is added to the cannabis extract at a rate of about 400 mL of solvent per about 1,000 g of extract. In some embodiments, acetonitrile is added to the cannabis extract at a rate of about 410 mL of solvent per about 1,000 g of extract. In some embodiments, acetonitrile is added to the cannabis extract at a rate of about 420 mL of solvent per about 1,000 g of extract. In some embodiments, acetonitrile is added to the cannabis extract at a rate of about 430 mL of solvent per about 1,000 g of extract. In some embodiments, acetonitrile is added to the cannabis extract at a rate of about 440 mL of solvent per about 1,000 g of extract. In some embodiments, acetonitrile is added to the cannabis extract at a rate of about 450 mL of solvent per about 1,000 g of extract. In some embodiments, acetonitrile is added to the cannabis extract at a rate of about 460 mL of solvent per about 1,000 g of extract. In some embodiments, acetonitrile is added to the cannabis extract at a rate of about 470 mL of solvent per about 1,000 g of extract. In some embodiments, acetonitrile is added to the cannabis extract at a rate of about 480 mL of solvent per about 1,000 g of extract. In some embodiments, acetonitrile is added to the cannabis extract at a rate of about 490 mL of solvent per about 1,000 g of extract. In some embodiments, acetonitrile is added to the cannabis extract at a rate of about 500 mL of solvent per about 1,000 g of extract. In some embodiments, acetonitrile is added to the cannabis extract at a rate of about 510 mL of solvent per about 1,000 g of extract. In some embodiments, acetonitrile is added to the cannabis extract at a rate of about 520 mL of solvent per about 1,000 g of extract. In some embodiments, acetonitrile is added to the cannabis extract at a rate of about 530 mL of solvent per about 1,000 g of extract. In some embodiments, acetonitrile is added to the cannabis extract at a rate of about 540 mL of solvent per about 1,000 g of extract. In some embodiments, acetonitrile is added to the cannabis extract at a rate of about 550 mL of solvent per about 1,000 g of extract. In some embodiments, acetonitrile is added to the cannabis extract at a rate of about 560 mL of solvent per about 1,000 g of extract. In some embodiments, acetonitrile is added to the cannabis extract at a rate of about 570 mL of solvent per about 1,000 g of extract. In some embodiments, acetonitrile is added to the cannabis extract at a rate of about 580 mL of solvent per about 1,000 g of extract. In some embodiments, acetonitrile is added to the cannabis extract at a rate of about 590 mL of solvent per about 1,000 g of extract In some embodiments, acetonitrile is added to the cannabis extract at a rate of about 600 mL of solvent per about 1,000 g of extract. In some embodiments, acetonitrile is added to the cannabis extract at a rate of about 610 mL of solvent per about 1,000 g of extract. In some embodiments, acetonitrile is added to the cannabis extract at a rate of about 620 mL of solvent per about 1,000 g of extract. In some embodiments, acetonitrile is added to the cannabis extract at a rate of about 630 mL of solvent per about 1,000 g of extract. In some embodiments, acetonitrile is added to the cannabis extract at a rate of about 640 mL of solvent per about 1,000 g of extract. In some embodiments, acetonitrile is added to the cannabis extract at a rate of about 650 mL of solvent per about 1,000 g of extract. In some embodiments, acetonitrile is added to the cannabis extract at a rate of about 660 mL of solvent per about 1,000 g of extract. In some embodiments, acetonitrile is added to the cannabis extract at a rate of about 560 mL of solvent per about 1,000 g of extract. In some embodiments, acetonitrile is added to the cannabis extract at a rate of about 680 mL of solvent per about 1,000 g of extract. In some embodiments, acetonitrile is added to the cannabis extract at a rate of about 690 mL of solvent per about 1,000 g of extract In some embodiments, acetonitrile is added to the cannabis extract at a rate of about 700 mL of solvent per about 1,000 g of extract. In some embodiments, acetonitrile is added to the cannabis extract at a rate of about 710 mL of solvent per about 1,000 g of extract. In some embodiments, acetonitrile is added to the cannabis extract at a rate of about 720 mL of solvent per about 1,000 g of extract. In some embodiments, acetonitrile is added to the cannabis extract at a rate of about 730 mL of solvent per about 1,000 g of extract. In some embodiments, acetonitrile is added to the cannabis extract at a rate of about 740 mL of solvent per about 1,000 g of extract. In some embodiments, acetonitrile is added to the cannabis extract at a rate of about 750 mL of solvent per about 1,000 g of extract. In some embodiments, acetonitrile is added to the cannabis extract at a rate of about 760 mL of solvent per about 1,000 g of extract. In some embodiments, acetonitrile is added to the cannabis extract at a rate of about 770 mL of solvent per about 1,000 g of extract. In some embodiments, acetonitrile is added to the cannabis extract at a rate of about 780 mL of solvent per about 1,000 g of extract. In some embodiments, acetonitrile is added to the cannabis extract at a rate of about 790 mL of solvent per about 1,000 g of extract In some embodiments, acetonitrile is added to the cannabis extract at a rate of about 800 mL of solvent per about 1,000 g of extract. In some embodiments, acetonitrile is added to the cannabis extract at a rate of about 810 mL of solvent per about 1,000 g of extract. In some embodiments, acetonitrile is added to the cannabis extract at a rate of about 820 mL of solvent per about 1,000 g of extract. In some embodiments, acetonitrile is added to the cannabis extract at a rate of about 830 mL of solvent per about 1,000 g of extract. In some embodiments, acetonitrile is added to the cannabis extract at a rate of about 840 mL of solvent per about 1,000 g of extract. In some embodiments, acetonitrile is added to the cannabis extract at a rate of about 850 mL of solvent per about 1,000 g of extract. In some embodiments, acetonitrile is added to the cannabis extract at a rate of about 860 mL of solvent per about 1,000 g of extract. In some embodiments, acetonitrile is added to the cannabis extract at a rate of about 870 mL of solvent per about 1,000 g of extract. In some embodiments, acetonitrile is added to the cannabis extract at a rate of about 880 mL of solvent per about 1,000 g of extract. In some embodiments, acetonitrile is added to the cannabis extract at a rate of about 890 mL of solvent per about 1,000 g of extract In some embodiments, acetonitrile is added to the cannabis extract at a rate of about 900 mL of solvent per about 1,000 g of extract. In some embodiments, acetonitrile is added to the cannabis extract at a rate of about 910 mL of solvent per about 1,000 g of extract. In some embodiments, acetonitrile is added to the cannabis extract at a rate of about 920 mL of solvent per about 1,000 g of extract. In some embodiments, acetonitrile is added to the cannabis extract at a rate of about 930 mL of solvent per about 1,000 g of extract. In some embodiments, acetonitrile is added to the cannabis extract at a rate of about 940 mL of solvent per about 1,000 g of extract. In some embodiments, acetonitrile is added to the cannabis extract at a rate of about 950 mL of solvent per about 1,000 g of extract. In some embodiments, acetonitrile is added to the cannabis extract at a rate of about 960 mL of solvent per about 1,000 g of extract. In some embodiments, acetonitrile is added to the cannabis extract at a rate of about 970 mL of solvent per about 1,000 g of extract. In some embodiments, acetonitrile is added to the cannabis extract at a rate of about 980 mL of solvent per about 1,000 g of extract. In some embodiments, acetonitrile is added to the cannabis extract at a rate of about 990 mL of solvent per about 1,000 g of extract In some embodiments, acetonitrile is added to the cannabis extract at a rate of about 1,000 mL of solvent per about 1,000 g of extract. In some embodiments, acetonitrile is added to the cannabis extract at a rate below about 200 mL of solvent per about 1,000 g of extract. In some embodiments, acetonitrile is added to the cannabis extract at a rate above about 1,000 mL of solvent per about 1,000 g of extract. In some embodiments, acetonitrile is added to the cannabis extract at a rate of between about 300 mL to about 600 mL of solvent per about 1,000 g of extract. In some embodiments, acetonitrile is added to the cannabis extract at a rate of between about 350 mL to about 450 mL of solvent per about 1,000 g of extract.

In some embodiments, the amount of solvent used is the minimum amount necessary to allow for suspension and circulation of the slurry. In some embodiments, additional solvent may need to be added as further cannabinoid acids crystallize out of solution. In some embodiments, the amount of solvent, as measured in mL, is between about 100 mL to about 20,000 mL, including about 100 mL, about 200 mL, about 300 mL, about 400 mL, about 500 mL, about 600 mL, about 700 mL, about 800 mL, about 900 mL, about 1,000 mL, about 1,100 mL, about 1,200 mL, about 1,300 mL, about 1,400 mL, about 1,500 mL, about 1,600 mL, about 1,700 mL, about 1,800 mL, about 1,900 mL, about 2,000 mL, about 2,100 mL, about 2,200 mL, about 2,300 mL, about 2,400 mL, about 2,500 mL, about 2,600 mL, about 2,700 mL, about 2,800 mL, about 2,900 mL, about 3,000 mL, about 3,100 mL, about 3,200 mL, about 3,300 mL, about 3,400 mL, about 3,500 mL, about 3,600 mL, about 3,700 mL, about 3,800 mL, about 3,900 mL, about 4,000 mL, about 4,100 mL, about 4,200 mL, about 4,300 mL, about 4,400 mL, about 4,500 mL, about 4,600 mL, about 4,700 mL, about 4,800 mL, about 4,900 mL, about 5,000 mL, about 5,100 mL, about 5,200 mL, about 5,300 mL, about 5,400 mL, about 5,500 mL, about 5,600 mL, about 5,700 mL, about 5,800 mL, about 5,900 mL, about 6,000 mL, about 6,100 mL, about 6,200 mL, about 6,300 mL, about 6,400 mL, about 6,500 mL, about 6,600 mL, about 6,700 mL, about 6,800 mL, about 6,900 mL, about 7,000 mL, about 7,100 mL, about 7,200 mL, about 7,300 mL, about 7,400 mL, about 7,500 mL, about 7,600 mL, about 7,700 mL, about 7,800 mL, about 7,900 mL, about 8,000 mL, about 8,100 mL, about 8,200 mL, about 8,300 mL, about 8,400 mL, about 8,500 mL, about 8,600 mL, about 8,700 mL, about 8,800 mL, about 8,900 mL, about 9,000 mL, about 9,100 mL, about 9,200 mL, about 9,300 mL, about 9,400 mL, about 9,500 mL, about 9,600 mL, about 9,700 mL, about 9,800 mL, about 9,900 mL, about 10,000 mL, about 10,100 mL, about 10,200 mL, about 10,300 mL, about 10,400 mL, about 10,500 mL, about 10,600 mL, about 10,700 mL, about 10,800 mL, about 10,900 mL, about 11,000 mL, about 11,100 mL, about 11,200 mL, about 11,300 mL, about 11,400 mL, about 11,500 mL, about 11,600 mL, about 11,700 mL, about 11,800 mL, about 11,900 mL, about 12,000 mL, about 12,100 mL, about 12,200 mL, about 12,300 mL, about 12,400 mL, about 12,500 mL, about 12,600 mL, about 12,700 mL, about 12,800 mL, about 12,900 mL, about 13,000 mL, about 13,100 mL, about 13,200 mL, about 13,300 mL, about 13,400 mL, about 13,500 mL, about 13,600 mL, about 13,700 mL, about 13,800 mL, about 13,900 mL, about 14,000 mL, about 14,100 mL, about 14,200 mL, about 14,300 mL, about 14,400 mL, about 14,500 mL, about 14,600 mL, about 14,700 mL, about 14,800 mL, about 14,900 mL, about 15,000 mL, about 15,100 mL, about 15,200 mL, about 15,300 mL, about 15,400 mL, about 15,500 mL, about 15,600 mL, about 15,700 mL, about 15,800 mL, about 15,900 mL, about 16,000 mL, about 16,100 mL, about 16,200 mL, about 16,300 mL, about 16,400 mL, about 16,500 mL, about 16,600 mL, about 16,700 mL, about 16,800 mL, about 16,900 mL, about 17,000 mL, about 17,100 mL, about 17,200 mL, about 17,300 mL, about 17,400 mL, about 17,500 mL, about 17,600 mL, about 17,700 mL, about 17,800 mL, about 17,900 mL, about 18,000 mL, about 18,100 mL, about 18,200 mL, about 18,300 mL, about 18,400 mL, about 18,500 mL, about 18,600 mL, about 18,700 mL, about 18,800 mL, about 18,900 mL, about 19,000 mL, about 19,100 mL, about 19,200 mL, about 19,300 mL, about 19,400 mL, about 19,500 mL, about 19,600 mL, about 19,700 mL, about 19,800 mL, about 19,900 mL, about 20,000 mL, and values in between, as would be appreciated by one of skill. In other embodiments, and as would be recognized by those of skill, where vessel sizes and/or batch sizes are larger, the amount of solvent is greater than about 20 L (i.e., 20,000 mL), such as greater than about 30 L, greater than about 40 L, greater than about 50 L, or greater than about 100 L.

In some embodiments, the amount of cannabis extract necessary for the invention is between about 500 g, to about 50,000 g. In some embodiments, the cannabis extract necessary for the invention is about 500 g, about 600 g, about 700 g, about 800 g, about 900 g, about 1,000 g, about 1,100 g, about 1,200 g, about 1,300 g, about 1,400 g, about 1,500 g, about 1,600 g, about 1,700 g, about 1,800 g, about 1,900 g, about 2,000 g, about 2,100 g, about 2,200 g, about 2,300 g, about 2,400 g, about 2,500 g, about 2,600 g, about 2,700 g, about 2,800 g, about 2,900 g, about 3,000 g, about 3,100 g, about 3,200 g, about 3,300 g, about 3,400 g, about 3,500 g, about 3,600 g, about 3,700 g, about 3,800 g, about 3,900 g, about 4,000 g, about 4,100 g, about 4,200 g, about 4,300 g, about 4,400 g, about 4,500 g, about 4,600 g, about 4,700 g, about 4,800 g, about 4,900 g, about 5,000 g, about 5,100 g, about 5,200 g, about 5,300 g, about 5,400 g, about 5,500 g, about 5,600 g, about 5,700 g, about 5,800 g, about 5,900 g, about 6,000 g, about 6,100 g, about 6,200 g, about 6,300 g, about 6,400 g, about 6,500 g, about 6,600 g, about 6,700 g, about 6,800 g, about 6,900 g, about 7,000 g, about 7,100 g, about 7,200 g, about 7,300 g, about 7,400 g, about 7,500 g, about 7,600 g, about 7,700 g, about 7,800 g, about 7,900 g, about 8,000 g, about 8,100 g, about 8,200 g, about 8,300 g, about 8,400 g, about 8,500 g, about 8,600 g, about 8,700 g, about 8,800 g, about 8,900 g, about 9,000 g, about 9,100 g, about 9,200 g, about 9,300 g, about 9,400 g, about 9,500 g, about 9,600 g, about 9,700 g, about 9,800 g, about 9,900 g, about 10,000 g, about 10,100 g, about 10,200 g, about 10,300 g, about 10,400 g, about 10,500 g, about 10,600 g, about 10,700 g, about 10,800 g, about 10,900 g, about 11,000 g, about 11,100 g, about 11,200 g, about 11,300 g, about 11,400 g, about 11,500 g, about 11,600 g, about 11,700 g, about 11,800 g, about 11,900 g, about 12,000 g, about 12,100 g, about 12,200 g, about 12,300 g, about 12,400 g, about 12,500 g, about 12,600 g, about 12,700 g, about 12,800 g, about 12,900 g, about 13,000 g, about 13,100 g, about 13,200 g, about 13,300 g, about 13,400 g, about 13,500 g, about 13,600 g, about 13,700 g, about 13,800 g, about 13,900 g, about 14,000 g, about 14,100 g, about 14,200 g, about 14,300 g, about 14,400 g, about 14,500 g, about 14,600 g, about 14,700 g, about 14,800 g, about 14,900 g, about 15,000 g, about 15,100 g, about 15,200 g, about 15,300 g, about 15,400 g, about 15,500 g, about 15,600 g, about 15,700 g, about 15,800 g, about 15,900 g, about 16,000 g, about 16,100 g, about 16,200 g, about 16,300 g, about 16,400 g, about 16,500 g, about 16,600 g, about 16,700 g, about 16,800 g, about 16,900 g, about 17,000 g, about 17,100 g, about 17,200 g, about 17,300 g, about 17,400 g, about 17,500 g, about 17,600 g, about 17,700 g, about 17,800 g, about 17,900 g, about 18,000 g, about 18,100 g, about 18,200 g, about 18,300 g, about 18,400 g, about 18,500 g, about 18,600 g, about 18,700 g, about 18,800 g, about 18,900 g, about 19,000 g, about 19,100 g, about 19,200 g, about 19,300 g, about 19,400 g, about 19,500 g, about 19,600 g, about 19,700 g, about 19,800 g, about 19,900 g, about 20,000 g, about 20,100 g, about 20,200 g, about 20,300 g, about 20,400 g, about 20,500 g, about 20,600 g, about 20,700 g, about 20,800 g, about 20,900 g, about 21,000 g, about 21,100 g, about 21,200 g, about 21,300 g, about 21,400 g, about 21,500 g, about 21,600 g, about 21,700 g, about 21,800 g, about 21,900 g, about 22,000 g, about 22,100 g, about 22,200 g, about 22,300 g, about 22,400 g, about 22,500 g, about 22,600 g, about 22,700 g, about 22,800 g, about 22,900 g, about 23,000 g, about 23,100 g, about 23,200 g, about 23,300 g, about 23,400 g, about 23,500 g, about 23,600 g, about 23,700 g, about 23,800 g, about 23,900 g, about 24,000 g, about 24,100 g, about 24,200 g, about 24,300 g, about 24,400 g, about 24,500 g, about 24,600 g, about 24,700 g, about 24,800 g, about 24,900 g, about 25,000 g, about 25,100 g, about 25,200 g, about 25,300 g, about 25,400 g, about 25,500 g, about 25,600 g, about 25,700 g, about 25,800 g, about 25,900 g, about 26,000 g, about 26,100 g, about 26,200 g, about 26,300 g, about 26,400 g, about 26,500 g, about 26,600 g, about 26,700 g, about 26,800 g, about 26,900 g, about 27,000 g, about 27,100 g, about 27,200 g, about 27,300 g, about 27,400 g, about 27,500 g, about 27,600 g, about 27,700 g, about 27,800 g, about 27,900 g, about 28,000 g, about 28,100 g, about 28,200 g, about 28,300 g, about 28,400 g, about 28,500 g, about 28,600 g, about 28,700 g, about 28,800 g, about 28,900 g, about 29,000 g, about 29,100 g, about 29,200 g, about 29,300 g, about 29,400 g, about 29,500 g, about 29,600 g, about 29,700 g, about 29,800 g, about 29,900 g, about 30,000 g, about 30,100 g, about 30,200 g, about 30,300 g, about 30,400 g, about 30,500 g, about 30,600 g, about 30,700 g, about 30,800 g, about 30,900 g, about 31,000 g, about 31,100 g, about 31,200 g, about 31,300 g, about 31,400 g, about 31,500 g, about 31,600 g, about 31,700 g, about 31,800 g, about 31,900 g, about 32,000 g, about 32,100 g, about 32,200 g, about 32,300 g, about 32,400 g, about 32,500 g, about 32,600 g, about 32,700 g, about 32,800 g, about 32,900 g, about 33,000 g, about 33,100 g, about 33,200 g, about 33,300 g, about 33,400 g, about 33,500 g, about 33,600 g, about 33,700 g, about 33,800 g, about 33,900 g, about 34,000 g, about 34,100 g, about 34,200 g, about 34,300 g, about 34,400 g, about 34,500 g, about 34,600 g, about 34,700 g, about 34,800 g, about 34,900 g, about 35,000 g, about 35,100 g, about 35,200 g, about 35,300 g, about 35,400 g, about 35,500 g, about 35,600 g, about 35,700 g, about 35,800 g, about 35,900 g, about 36,000 g, about 36,100 g, about 36,200 g, about 36,300 g, about 36,400 g, about 36,500 g, about 36,600 g, about 36,700 g, about 36,800 g, about 36,900 g, about 37,000 g, about 37,100 g, about 37,200 g, about 37,300 g, about 37,400 g, about 37,500 g, about 37,600 g, about 37,700 g, about 37,800 g, about 37,900 g, about 38,000 g, about 38,100 g, about 38,200 g, about 38,300 g, about 38,400 g, about 38,500 g, about 38,600 g, about 38,700 g, about 38,800 g, about 38,900 g, about 39,000 g, about 39,100 g, about 39,200 g, about 39,300 g, about 39,400 g, about 39,500 g, about 39,600 g, about 39,700 g, about 39,800 g, about 39,900 g, about 40,000 g, about 40,100 g, about 40,200 g, about 40,300 g, about 40,400 g, about 40,500 g, about 40,600 g, about 40,700 g, about 40,800 g, about 40,900 g, about 41,000 g, about 41,100 g, about 41,200 g, about 41,300 g, about 41,400 g, about 41,500 g, about 41,600 g, about 41,700 g, about 41,800 g, about 41,900 g, about 42,000 g, about 42,100 g, about 42,200 g, about 42,300 g, about 42,400 g, about 42,500 g, about 42,600 g, about 42,700 g, about 42,800 g, about 42,900 g, about 43,000 g, about 43,100 g, about 43,200 g, about 43,300 g, about 43,400 g, about 43,500 g, about 43,600 g, about 43,700 g, about 43,800 g, about 43,900 g, about 44,000 g, about 44,100 g, about 44,200 g, about 44,300 g, about 44,400 g, about 44,500 g, about 44,600 g, about 44,700 g, about 44,800 g, about 44,900 g, about 45,000 g, about 45,100 g, about 45,200 g, about 45,300 g, about 45,400 g, about 45,500 g, about 45,600 g, about 45,700 g, about 45,800 g, about 45,900 g, about 46,000 g, about 46,100 g, about 46,200 g, about 46,300 g, about 46,400 g, about 46,500 g, about 46,600 g, about 46,700 g, about 46,800 g, about 46,900 g, about 47,000 g, about 47,100 g, about 47,200 g, about 47,300 g, about 47,400 g, about 47,500 g, about 47,600 g, about 47,700 g, about 47,800 g, about 47,900 g, about 48,000 g, about 48,100 g, about 48,200 g, about 48,300 g, about 48,400 g, about 48,500 g, about 48,600 g, about 48,700 g, about 48,800 g, about 48,900 g, about 49,000 g, about 49,100 g, about 49,200 g, about 49,300 g, about 49,400 g, about 49,500 g, about 49,600 g, about 49,700 g, about 49,800 g, about 49,900 g, about 50,000 g, and values in between, as would be immediately apparent to one of skill, and wherein the cannabis is added with the appropriate amount of solvent, as stated in the proportion herein. In some embodiments, the amount of cannabis extract necessary for the invention is greater than 50,000 g, including about 60,000 g, about 75,000 g, about 90,000 g, and, in some embodiments, greater than 100,000 g. In all cases, however, the cannabis extract is added with the appropriate amount of solvent, as stated in the proportion herein.

In some embodiments, processing times will span from about 10 minutes to about 36 hours, including about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 90 minutes, about 120 minutes, about 150 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, and times in between as would be immediately apparent to one of skill. Required processing times may be dependent on, e.g., blade shape, stirring speed, and temperature of slurry, as will be recognized by those of skill, and determined using the practice of ordinary skill, based on the teachings herein and general knowledge in the art without significant experimentation.

In some embodiments, additional solvent will be added as further cannabinoid acids crystallize out of solution (i.e., as further cannabinoid acids precipitate). In such embodiments, whether or not additional solvent is needed for agitation will be readily apparent to one of skill. However, to ensure those of skill are enabled to carry out the process of the invention, additional solvent should be added if mixing is insufficient, e.g., where mixing during the agitation step is not proceeding in both a vertical and a horizontal manner—e.g., if the solution is not fluidly intermixing, such that the material towards the top of the fluid column is not freely mixing with the material towards the bottom of the fluid column. In some embodiments, this will be evident by sufficient mixing adjacent to the mixing means (e.g., a paddle, a gate, a blade, or any other substantially solid material useful in mixing a solution), but not elsewhere in the solution. If a practitioner determines additional solvent is necessary, because top to bottom intermixing is not sufficiently occurring, additional solvent may be added in increments of, e.g., 1/10th of the initial total solvent, or in other small increments, such as between about ⅕th to about 1/20th, or from 1/20th to 1/50th.

Thus, in some embodiments, wherein additional solvent is required to ensure sufficient mixing of the solution, and about 100 mL of solvent was originally combined with the cannabis extract, additional solvent would be added in about 10 mL increments; in some embodiments, wherein additional solvent is required to ensure sufficient mixing of the solution, and about 200 mL of solvent was originally combined with the cannabis extract, additional solvent would be added in about 20 mL increments; in some embodiments, wherein additional solvent is required to ensure sufficient mixing of the solution, and about 300 mL of solvent was originally combined with the cannabis extract, additional solvent would be added in about 30 mL increments; in some embodiments, wherein additional solvent is required to ensure sufficient mixing of the solution, and about 400 mL of solvent was originally combined with the cannabis extract, additional solvent would be added in about 40 mL increments; in some embodiments, wherein additional solvent is required to ensure sufficient mixing of the solution, and about 500 mL of solvent was originally combined with the cannabis extract, additional solvent would be added in about 50 mL increments; in some embodiments, wherein additional solvent is required to ensure sufficient mixing of the solution, and about 600 mL of solvent was originally combined with the cannabis extract, additional solvent would be added in about 60 mL increments; in some embodiments, wherein additional solvent is required to ensure sufficient mixing of the solution, and about 700 mL of solvent was originally combined with the cannabis extract, additional solvent would be added in about 70 mL increments; in some embodiments, wherein additional solvent is required to ensure sufficient mixing of the solution, and about 800 mL of solvent was originally combined with the cannabis extract, additional solvent would be added in about 80 mL increments; in some embodiments, wherein additional solvent is required to ensure sufficient mixing of the solution, and about 900 mL of solvent was originally combined with the cannabis extract, additional solvent would be added in about 90 mL increments; in some embodiments, wherein additional solvent is required to ensure sufficient mixing of the solution, and about 1,000 mL of solvent was originally combined with the cannabis extract, additional solvent would be added in about 100 mL increments; in some embodiments, wherein additional solvent is required to ensure sufficient mixing of the solution, and about 1,500 mL of solvent was originally combined with the cannabis extract, additional solvent would be added in about 150 mL increments; in some embodiments, wherein additional solvent is required to ensure sufficient mixing of the solution, and about 2,000 mL of solvent was originally combined with the cannabis extract, additional solvent would be added in about 200 mL increments; in some embodiments, wherein additional solvent is required to ensure sufficient mixing of the solution, and about 2,500 mL of solvent was originally combined with the cannabis extract, additional solvent would be added in about 250 mL increments; in some embodiments, wherein additional solvent is required to ensure sufficient mixing of the solution, and about 3,000 mL of solvent was originally combined with the cannabis extract, additional solvent would be added in about 300 mL increments; in some embodiments, wherein additional solvent is required to ensure sufficient mixing of the solution, and about 3,500 mL of solvent was originally combined with the cannabis extract, additional solvent would be added in about 350 mL increments; in some embodiments, wherein additional solvent is required to ensure sufficient mixing of the solution, and about 4,000 mL of solvent was originally combined with the cannabis extract, additional solvent would be added in about 400 mL increments; in some embodiments, wherein additional solvent is required to ensure sufficient mixing of the solution, and about 4,500 mL of solvent was originally combined with the cannabis extract, additional solvent would be added in about 450 mL increments; in some embodiments, wherein additional solvent is required to ensure sufficient mixing of the solution, and about 5,000 mL of solvent was originally combined with the cannabis extract, additional solvent would be added in about 500 mL increments. Likewise, in some embodiments, wherein additional solvent is required to ensure sufficient mixing of the solution, and more than about 5,000 mL of solvent was originally combined with the cannabis extract, additional solvent would be added in increments of 1/10th of the initial total solvent, as would be apparent to one of skill. Where additional solvent is added in other than increments of 1/10th of the initial total solvent, such as between about ⅕th to about 1/20th, or from 1/20th to 1/50th of the initial total solvent, the amount of additional solvent will be readily determinable as above. In such embodiments, the addition of solvent may continue until sufficient intermixing is realized, such that material towards the top of the fluid column or other container is freely mixing with material towards the bottom of the fluid column or other container, such that a homogeneous slurry or a substantially homogeneous slurry can be obtained.

In some embodiments, both the slurry and clean solvent washes are maintained at a temperature of about 0° C. or less, (including about −1° C. or less, −3° C. or less, −5° C. or less, −10° C. or less, −15° C. or less, −25° C. or less, −35° C. or less, −50° C. or less, −75° C. or less, and about −100° C. or less). In other embodiments, both the slurry and clean solvent washes are maintained at a temperature above about 1° C., above about 2° C., above about 3° C., above about 4° C., above about 5° C., above about 6° C., above about 7° C., above about 8° C., above about 9° C., above about 10° C., above about 11° C., above about 12° C., above about 13° C., above about 14° C., above about 15° C., above about 16° C., above about 17° C., above about 18° C., above about 19° C., above about 20° C., above about 21° C., above about 22° C., above about 23° C., above about 24° C., above about 25° C., above about 26° C., above about 27° C., above about 28° C., above about 29° C., above about 30° C., above about 31° C., above about 32° C., above about 33° C., above about 34° C., above about 35° C., above about 36° C., above about 37° C., above about 38° C., above about 39° C., above about 40° C., above about 41° C., above about 42° C., above about 43° C., above about 44° C., above about 45° C., above about 46° C., above about 47° C., above about 48° C., above about 49° C., above about 50° C., above about 51° C., above about 52° C., above about 53° C., above about 54° C., above about 55° C., above about 56° C., above about 57° C., above about 58° C., above about 59° C., above about 60° C., above about 61° C., above about 62° C., above about 63° C., above about 64° C., above about 65° C., above about 66° C., above about 67° C., above about 68° C., above about 69° C., above about 70° C., above about 71° C., above about 72° C., above about 73° C., above about 74° C., above about 75° C., above about 76° C., above about 77° C., above about 78° C., above about 79° C., above about 80° C., above about 81° C., above about 82° C., above about 83° C., above about 84° C., above about 85° C., above about 86° C., above about 87° C., above about 88° C., above about 89° C., above about 90° C., above about 91° C., above about 92° C., above about 93° C., above about 94° C., above about 95° C., above about 96° C., above about 97° C., above about 98° C., above about 99° C., above about 100° C., above about 101° C., above about 102° C., above about 103° C., above about 104° C., and values in between, as would be appreciated by those of skill.

In some embodiments, temperatures of the slurry and clean solvent washes below about 10° C. may cause unwanted compounds to not dissolve and/or to precipitate out of solution, which may negatively affect purity and appearance of cannabinoid acid precipitate. Accordingly, in some embodiments, both the slurry and clean solvent washes are maintained at a temperature above about 10° C., above about 11° C., above about 12° C., above about 13° C., above about 14° C., above about 15° C., above about 16° C., above about 17° C., above about 18° C., above about 19° C., above about 20° C., above about 21° C., above about 22° C., above about 23° C., above about 24° C., and above about 25° C. However, in other embodiments, it has been surprisingly discovered that maintaining temperatures of the slurry and clean solvent washes below −40° C. are optimal. Accordingly, in some embodiments, both the slurry and clean solvent washes are maintained at a temperature below −40° C., including about −45° C. or less, about −50° C. or less, about −75° C. or less, and about −100° C. or less.

In some embodiments, temperatures of the slurry and clean solvent washes above about 20° C. may cause an increase in the amount of cannabinoid acids dissolved in solution, which may negatively affect the final yield of purified cannabinoid acids. However, in other embodiments, it has been surprisingly discovered that maintaining temperatures of the slurry and clean solvent washes above 25° C. are optimal. Accordingly, in some embodiments, both the slurry and clean solvent washes are maintained at a temperature above 25° C., including above about 26° C., above about 27° C., above about 28° C., above about 29° C., above about 30° C., above about 31° C., above about 32° C., above about 33° C., above about 34° C., above about 35° C., above about 36° C., above about 37° C., above about 38° C., above about 39° C., above about 40° C., above about 41° C., above about 42° C., above about 43° C., above about 44° C., above about 45° C., above about 46° C., above about 47° C., above about 48° C., above about 49° C., above about 50° C., above about 51° C., above about 52° C., above about 53° C., above about 54° C., above about 55° C., above about 56° C., above about 57° C., above about 58° C., above about 59° C., above about 60° C., above about 61° C., above about 62° C., above about 63° C., above about 64° C., above about 65° C., above about 66° C., above about 67° C., above about 68° C., above about 69° C., above about 70° C., above about 71° C., above about 72° C., above about 73° C., above about 74° C., above about 75° C., above about 76° C., above about 77° C., above about 78° C., above about 79° C., above about 80° C., above about 81° C., above about 82° C., above about 83° C., above about 84° C., above about 85° C., above about 86° C., above about 87° C., above about 88° C., above about 89° C., above about 90° C., above about 91° C., above about 92° C., above about 93° C., above about 94° C., above about 95° C., above about 96° C., above about 97° C., above about 98° C., above about 99° C., above about 100° C., above about 101° C., about 102° C., about 103° C., above about 104° C., and values in between, as would be appreciated by those of skill.

In some embodiments, both the slurry and clean solvent washes are maintained at a temperature between about 10° C. to about 20° C. In some embodiments, the slurry and clean solvent washes are maintained at a temperature of about 10° C., about 10.1° C., about 10.2° C., about 10.3° C., about 10.4° C., about 10.5° C., about 10.6° C., about 10.7° C., about 10.8° C., about 10.9° C., about 11° C., about 11.1° C., about 11.2° C., about 11.3° C., about 11.4° C., about 11.5° C., about 11.6° C., about 11.7° C., about 11.8° C., about 11.9° C., about 12° C., about 12.1° C., about 12.2° C., about 12.3° C., about 12.4° C., about 12.5° C., about 12.6° C., about 12.7° C., about 12.8° C., about 12.9° C., about 13° C., about 13.1° C., about 13.2° C., about 13.3° C., about 13.4° C., about 13.5° C., about 13.6° C., about 13.7° C., about 13.8° C., about 13.9° C., about 14° C., about 14.1° C., about 14.2° C., about 14.3° C., about 14.4° C., about 14.5° C., about 14.6° C., about 14.7° C., about 14.8° C., about 14.9° C., about 15° C., about 15.1° C., about 15.2° C., about 15.3° C., about 15.4° C., about 15.5° C., about 15.6° C., about 15.7° C., about 15.8° C., about 15.9° C., about 16° C., about 16.1° C., about 16.2° C., about 16.3° C., about 16.4° C., about 16.5° C., about 16.6° C., about 16.7° C., about 16.8° C., about 16.9° C., about 17° C., about 17.1° C., about 17.2° C., about 17.3° C., about 17.4° C., about 17.5° C., about 17.6° C., about 17.7° C., about 17.8° C., about 17.9° C., about 18° C., about 18.1° C., about 18.2° C., about 18.3° C., about 18.4° C., about 18.5° C., about 18.6° C., about 18.7° C., about 18.8° C., about 18.9° C., about 19° C., about 19.1° C., about 19.2° C., about 19.3° C., about 19.4° C., about 19.5° C., about 19.6° C., about 19.7° C., about 19.8° C., about 19.9° C., about 20° C., and values in between, as would be appreciated by those of skill.

In step 103, step 102 continues until all visible chunks of extract material have been broken down into a homogeneous slurry, as defined herein, and cannabinoid acids have substantially ceased precipitating. As would be evident to one of skill, precipitation of cannabinoid acids proceeds in a nearly parabolic manner-meaning, the rate of precipitation increases until it reaches a peak, and then begins to decrease. In some embodiments of the invention disclosed herein, agitation is ceased once the rate of observable precipitation has slowed (i.e., after the rate of precipitation reaches a peak and then begins to decrease in rate) to the degree that no observable changes in cannabinoid acid precipitation occur for between about 30 minutes to about 60 minutes, including about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, and values in between, as would be apparent to one of skill.

Because the precipitation of cannabinoid acids necessarily thickens the slurry, in such embodiments, “observable changes in cannabinoid precipitation” refers to observable changes in the slurry's thickness. Note, however, as would be immediately apparent to one of skill, while precipitation may have “substantially ceased,” precipitation may still occur-albeit in low amounts, at low rates-after agitation ceases. In some embodiments, the time at which it takes for precipitation to cease is a function of temperature. In some embodiments, wherein the temperature remains constant throughout the agitation phase, the time at which it takes for precipitation to substantially cease is predictable, and should be in the knowledge of one of skill. In such embodiments, the lower the temperature, the quicker the cannabinoid acids will precipitate. As non-limiting examples, at about 20° C., one should expect the cannabinoid acids to have substantially ceased precipitating within about 24 hours (1,440 minutes); at about 5° C., one should expect the cannabinoid acids to have substantially ceased precipitating within about 12 hours (720 minutes); and at about 0° C., one should expect the cannabinoid acids to have substantially ceased precipitating within about 8 hours (480 minutes). In some embodiments, wherein temperature fluctuates throughout the process, precipitation will occur more rapidly as the temperature is decreased from about 20° C., to about 0° C. Thus, when the temperature is increased while precipitation is occurring, one should assume more time will be required for precipitation to substantially cease, while decreasing the temperature should have the opposite effects-decreasing the time at which precipitation will substantially cease.

As mentioned, precipitation of cannabinoid acids proceeds in a nearly parabolic manner-meaning, the rate of precipitation increases until it reaches a peak, and then begins to decrease. Because precipitation is observed as a thickening of the slurry, and the “thickness” of a liquid restricts its ease of flow, and a substance's resistance to motion under an applied force is measured as its “viscosity,” one may measure change of the viscosity of the slurry to determine when substantially no change in viscosity is occurring, to determine when precipitation has substantially ceased. In some embodiments, the reduction in the rate of precipitation can be objectively measured through the use of a viscometer, such that agitation is ceased when measurements collected from the viscometer illustrate that precipitation has substantially ceased. Herein, a “viscometer” is a device capable of, and useful for, measuring the viscosity of a fluid. Viscometers may either be used as stationary devices, wherein the liquid flows at a given rate, or may themselves move through a liquid at a given rate. Exemplary viscometers, as would be apparent to one of skill, include but are not limited to u-tube viscometers, falling-sphere viscometers, falling-piston viscometers, oscillating-piston viscometers, vibrational viscometers, rotational viscometers, bubble viscometers, rectangular-slit viscometers, krebs viscometers, and other such devices known to those of skill as being useful in measuring the viscosity of a fluid.

As it relates to the invention, in some embodiments, a viscometer, including but not limited to a u-tube viscometer, a falling-sphere viscometer, a falling-piston viscometer, an oscillating-piston viscometer, a vibrational viscometer, a rotational viscometer, a bubble viscometer, a rectangular-slit viscometer, a krebs viscometer, or any other such device, may be placed within the slurry during the agitation step to monitor the viscosity of the slurry (as measured in centipoise), wherein agitation is ceased once the viscosity remains substantially constant for between about 30 minutes, to about 60 minutes, including about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, and values in between, as would be apparent to one of skill.

Next, in step 104, the slurry is filtered using either positive pressure (on the slurry side) or negative pressure (on the filtrate side). Reference may also be had to FIG. 6, wherein at step 603 the slurry is filtered. Herein, “filtration,” in some embodiments, refers to a physical separation process useful for removing impurities or unwanted particles based on the size of said impurities or particles via a device having a pore size large enough to allow the desired contents to freely pass (i.e., larger than about the desired particle size), but small enough to prevent the unwanted impurities or particles from doing the same (i.e., smaller than about the unwanted particle size). In some embodiments, filtration refers to a process whereby solids are separated from a liquid by passage through a permeable membrane, wherein particles larger than the membrane's pores are retained, while the liquid (filtrate) passes through (Svarovski, 2000). Generally, filtration methods require the use of a “filter medium,” (e.g., the permeable membrane previously discussed), and a “force” exerted on the material desired to be filtered that facilitates its movement through the membrane. In some embodiments, the force may be any of, or a combination thereof, gravity, a vacuum, pressure, or centrifugal force, the specific embodiment being one that particularly suits the desired results of the practitioner, which will be apparent to one of skill (Svarovski, 2000).

More specifically, however, gravity filtration relies on atmospheric pressure to force the mixture/slurry desired to be filtered through the medium (Svarovski, 2000). Broadly, the slurry is fed into the top of the filter and through the medium, the aforementioned force of gravity (in the form of atmospheric pressure), driving the slurry through the medium. As it relates to vacuum filtration, the vacuum itself may be produced by the suction of a liquid pump, or a gas displacement device-including but not limited to a rotary vacuum pump or steam ejector (Svarovski, 2000). Regardless, the objective is to establish a force of up to about 83 kPa, including about 5 kPa, about 10 kPa, about 15 kPa, about 20 kPa, about 25 kPa, about 30 kPa, about 35 kPa, about 40 kPa, about 45 kPa, about 50 kPa, about 55 kPa, about 60 kPa, about 65 kPa, about 70 kPa, about 75 kPa, about 80 kPa, about 81 kPa, about 82 kPa, about 83 kPa, and values in between, as would be apparent to one of skill, behind the filter medium, which establishes a pressure differential that forces the slurry through the medium (Svarovski, 2000). Non-limiting examples of vacuum filters include belt, horizontal pan, vertical disc, and drum vacuum filters. Pressure filters, on the other hand, act similarly to an espresso machine, utilizing high pressures to force the slurry through the filter medium. Non-limiting examples of pressure filtration systems include plate-type filter presses, wherein a series of plates utilize a filter medium between them; case-enclosed pressure filters, which are comprised of filter-covered elements enclosed in a pressure tank; belt-type pressure filters, which utilize at least two belts that move in the same direction and, for a certain distance, share the same rollers with the slurry traveling between the same (the diameter of the rollers decreasing as the belt travels, which necessarily—although not to be bound by theory-subjects the slurry to a tremendous shear force as it travels over the roller (Svarovski, 2000). Lastly, centrifugal filtration systems utilize two types of separators-cyclones and centrifuges. The former has a simple, compact construction with low running costs, but has challenges filtering particles below about 10 μm, while the latter is more efficient at separating very fine particles (Svarovski, 2000). Regardless, centrifugal filtration is completed by passing the liquid under centrifugal force through a filter medium or perforated plate—the pore size of which is chosen by one of skill to achieve a desired result (Svarovski, 2000).

As it relates to the invention, filtration may be accomplished by any method known to one of skill as being useful in the process as disclosed, including but not limited to gravity filtration, vacuum filtration, non-limiting examples of which include filtration via a liquid pump, or a gas displacement device; pressure filtration, non-limiting examples of which include plate-type filter presses, case-enclosed pressure filters, and belt-type pressure filters; and/or centrifugal filtration, non-limiting examples of which include cyclone filters, and centrifuges. In some embodiments, “filtration” is completed by passing the slurry through a first filter screen, and then various filter chambers, wherein the first filter screen has a cut size of between about 110 microns to about 140 microns, and the subsequent filter chambers have a pore size of between about 70 microns, to about 100 microns. In some embodiments, the first filter screen is about 110 microns, about 111 microns, about 112 microns, about 113 microns, about 114 microns, about 115 microns, about 116 microns, about 117 microns, about 118 microns, about 119 microns, about 120 microns, about 121 microns, about 122 microns, about 123 microns, about 124 microns, about 125 microns, about 126 microns, about 127 microns, about 128 microns, about 129 microns, about 130 microns, about 131 microns, about 132 microns, about 133 microns, about 134 microns, about 135 microns, about 136 microns, about 137 microns, about 138 microns, about 139 microns, about 140 microns, or values in between as would be apparent to one of skill. In some embodiments, the subsequent filter chambers have a pore size of about 70 microns, about 71 microns, about 72 microns, about 73 microns, about 74 microns, about 75 microns, about 76 microns, about 77 microns, about 78 microns, about 79 microns, about 80 microns, about 81 microns, about 82 microns, about 83 microns, about 84 microns, about 85 microns, about 86 microns, about 87 microns, about 88 microns, about 89 microns, about 90 microns, about 91 microns, about 92 microns, about 93 microns, about 94 microns, about 95 microns, about 96 microns, about 97 microns, about 98 microns, about 99 microns, about 100 microns, and values in between, as would be apparent to one of skill. In some embodiments, “positive pressure,” refers to pressure within a system that exceeds the pressure of the surrounding system, while “negative pressure” refers to a system wherein the pressure of the system is less than the pressure of the surrounding system (Angstrom-Tech-Admin, 2016). In some embodiments, the positive pressure or negative pressure may be measured at between about 250 kPa, to about 460 kPa. In some embodiments, the positive or negative pressure may be about 250 kPa, about 255 kPa, about 260 kPa, about 265 kPa, about 270 kPa, about 275 kPa, about 280 kPa, about 285 kPa, about 290 kPa, about 295 kPa, about 300 kPa, about 305 kPa, about 310 kPa, about 315 kPa, about 320 kPa, about 325 kPa, about 330 kPa, about 335 kPa, about 340 kPa, about 345 kPa, about 350 kPa, about 355 kPa, about 360 kPa, about 365 kPa, about 370 kPa, about 375 kPa, about 380 kPa, about 385 kPa, about 390 kPa, about 395 kPa, about 400 kPa, about 405 kPa, about 410 kPa, about 415 kPa, about 420 kPa, about 425 kPa, about 430 kPa, about 435 kPa, about 440 kPa, about 445 kPa, about 450 kPa, about 455 kPa, about 460 kPa, and values in between, as would be apparent to one of skill.

In some embodiments, the filter cake is further washed with clean solvent (i.e., a fresh aprotic solvent, such as a solvent discussed herein, and including but not limited to acetonitrile), until visible color has been substantially removed. Note, as would be appreciated by one of skill, the volume of the clean solvent required for the clean solvent wash will be dependent on the extraction method utilized, the quality (including purity, coloration, etc.) of the extract, the shape of the blades used for stirring the slurry, stirring speed, and temperature of the slurry. That said, in some embodiments, the volume of clean solvent required for the clean solvent is between about 100 mL to about 1,000 mL, including about 100 mL, about 110 mL, about 120 mL, about 130 mL, about 140 mL, about 150 mL, about 160 mL, about 170 mL, about 180 mL, about 190 mL, about 200 mL, about 210 mL, about 220 mL, about 230 mL, about 240 mL, about 250 mL, about 260 mL, about 270 mL, about 280 mL, about 290 mL, about 300 mL, about 310 mL, about 320 mL, about 330 mL, about 340 mL, about 350 mL, about 360 mL, about 370 mL, about 380 mL, about 390 mL, about 400 mL, about 410 mL, about 420 mL, about 430 mL, about 440 mL, about 450 mL, about 460 mL, about 470 mL, about 480 mL, about 490 mL, about 500 mL, about 510 mL, about 520 mL, about 530 mL, about 540 mL, about 550 mL, about 560 mL, about 570 mL, about 580 mL, about 590 mL, about 600 mL, about 610 mL, about 620 mL, about 630 mL, about 640 mL, about 650 mL, about 660 mL, about 670 mL, about 680 mL, about 690 mL, about 700 mL, about 710 mL, about 720 mL, about 730 mL, about 740 mL, about 750 mL, about 760 mL, about 770 mL, about 780 mL, about 790 mL, about 800 mL, about 810 mL, about 820 mL, about 830 mL, about 840 mL, about 850 mL, about 860 mL, about 870 mL, about 880 mL, about 890 mL, about 900 mL, about 910 mL, about 920 mL, about 930 mL, about 940 mL, about 950 mL, about 960 mL, about 970 mL, about 980 mL, about 990 mL, about 1,000 mL, or values in between, as would be appreciated by one of skill.

In some embodiments, the temperature of the solvent used in the clean solvent wash is between about 10° C. to about 20° C. In some embodiments, the temperature of the clean solvent is about 10° C., about 10.1° C., about 10.2° C., about 10.3° C., about 10.4° C., about 10.5° C., about 10.6° C., about 10.7° C., about 10.8° C., about 10.9° C., about 11° C., about 11.1° C., about 11.2° C., about 11.3° C., about 11.4° C., about 11.5° C., about 11.6° C., about 11.7° C., about 11.8° C., about 11.9° C., about 12° C., about 12.1° C., about 12.2° C., about 12.3° C., about 12.4° C., about 12.5° C., about 12.6° C., about 12.7° C., about 12.8° C., about 12.9° C., about 13° C., about 13.1° C., about 13.2° C., about 13.3° C., about 13.4° C., about 13.5° C., about 13.6° C., about 13.7° C., about 13.8° C., about 13.9° C., about 14° C., about 14.1° C., about 14.2° C., about 14.3° C., about 14.4° C., about 14.5° C., about 14.6° C., about 14.7° C., about 14.8° C., about 14.9° C., about 15° C., about 15.1° C., about 15.2° C., about 15.3° C., about 15.4° C., about 15.5° C., about 15.6° C., about 15.7° C., about 15.8° C., about 15.9° C., about 16° C., about 16.1° C., about 16.2° C., about 16.3° C., about 16.4° C., about 16.5° C., about 16.6° C., about 16.7° C., about 16.8° C., about 16.9° C., about 17° C., about 17.1° C., about 17.2° C., about 17.3° C., about 17.4° C., about 17.5° C., about 17.6° C., about 17.7° C., about 17.8° C., about 17.9° C., about 18° C., about 18.1° C., about 18.2° C., about 18.3° C., about 18.4° C., about 18.5° C., about 18.6° C., about 18.7° C., about 18.8° C., about 18.9° C., about 19° C., about 19.1° C., about 19.2° C., about 19.3° C., about 19.4° C., about 19.5° C., about 19.6° C., about 19.7° C., about 19.8° C., about 19.9° C., about 20° C., and values in between, as would be appreciated by those of skill.

Next, in step 105, the remaining residual solvent is removed. As would be apparent to one of skill, solvent removal may, in some embodiments, take place in the container within which the mixing of step 102 (or of step 502 or step 602) took place; or, in other embodiments, within a processing vessel; or, in yet other embodiments, may take place in at least one separate container, including two containers, three containers, four containers, five containers, or more than five containers, wherein the fine crystalline material yielded in step 104 is transferred to the at least one separate container. Following step 105, and the removal of (at least, in some embodiments, a substantial amount of) remaining (residual) solvent, a cannabinoid acid precipitate is obtained. For example, reference may also be had to FIG. 5, wherein at step 503 the cannabinoid acid precipitate is obtained, and to FIG. 6, at step 604.

In such embodiments, wherein the fine crystalline material is transferred to at least one separate container, the separate container should contain a bottom, a top, at least one side, an interior area, and a means of accessing the interior area (including but not limited to a “lid” or a “hatch” and wherein the at least one side is operably connected to the bottom to form a substantially sealed container that prevents the leakage of the contents therein, which include but are not limited to solids, such as plant matter; liquids, such as liquid extracts, solvents, as defined herein; a homogeneous “slurry,” as defined herein; and gasses, such as those produced as a result of the chemical reactions of the inventions. As would be appreciated by those of skill, the at least one separate container may be constructed of any substantially solid material, including but limited to, glass, stainless steel, copper, or equivalent material useful in creating a container as disclosed herein. In such embodiments, once the fine crystalline material is transferred to the at least one separate container, the remaining residual solvent is removed.

In some embodiments, the remaining residual solvent is removed through the use of ambient evaporation, (i.e., evaporation completed at ambient temperatures), including at temperatures of between about 15° C. to about 35° C., including about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., and values in between as would be appreciated by one of skill. In some embodiments, ambient evaporation is conducted at temperatures above about 35° C. In some embodiments, ambient evaporation is conducted at temperatures below about 15° C. Note, in such embodiments, the means of accessing the interior area should remain open, so as to allow for sufficient aeration to facilitate evaporation.

In some embodiments, the remaining residual solvent is removed through the use of a vacuum oven at temperatures of between about 25° C. to about 45° C., including about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., and values in between as would be apparent to one of skill, and pressures below about 200 torr, including about 200 torr, 199 torr, about 198 torr, about 197 torr, about 196 torr, about 195 torr, about 194 torr, about 193 torr, about 192 torr, about 191 torr, about 190 torr, about 189 torr, about 188 torr, about 187 torr, about 186 torr, about 185 torr, about 184 torr, about 183 torr, about 182 torr, about 181 torr, about 180 torr, about 179 torr, about 178 torr, about 177 torr, about 176 torr, about 175 torr, about 174 torr, about 173 torr, about 172 torr, about 171 torr, about 170 torr, about 169 torr, about 168 torr, about 167 torr, about 166 torr, about 165 torr, about 164 torr, about 163 torr, about 162 torr, about 161 torr, about 160 torr, about 159 torr, about 158 torr, about 157 torr, about 156 torr, about 155 torr, about 154 torr, about 153 torr, about 152 torr, about 151 torr, about 150 torr, about 149 torr, about 148 torr, about 147 torr, about 146 torr, about 145 torr, about 144 torr, about 143 torr, about 142 torr, about 141 torr, about 140 torr, about 139 torr, about 138 torr, about 137 torr, about 136 torr, about 135 torr, about 134 torr, about 133 torr, about 132 torr, about 131 torr, about 130 torr, about 129 torr, about 128 torr, about 127 torr, about 126 torr, about 125 torr, about 124 torr, about 123 torr, about 122 torr, about 121 torr, about 120 torr, about 119 torr, about 118 torr, about 117 torr, about 116 torr, about 115 torr, about 114 torr, about 113 torr, about 112 torr, about 111 torr, about 110 torr, about 109 torr, about 108 torr, about 107 torr, about 106 torr, about 105 torr, about 104 torr, about 103 torr, about 102 torr, about 101 torr, about 100 torr, about 99 torr, about 98 torr, about 97 torr, about 96 torr, about 95 torr, about 94 torr, about 93 torr, about 92 torr, about 91 torr, about 90 torr, about 89 torr, about 88 torr, about 87 torr, about 86 torr, about 85 torr, about 84 torr, about 83 torr, about 82 torr, about 81 torr, about 80 torr, about 79 torr, about 78 torr, about 77 torr, about 76 torr, about 75 torr, about 74 torr, about 73 torr, about 72 torr, about 71 torr, about 70 torr, about 69 torr, about 68 torr, about 67 torr, about 66 torr, about 65 torr, about 64 torr, about 63 torr, about 62 torr, about 61 torr, about 60 torr, about 59 torr, about 58 torr, about 57 torr, about 56 torr, about 55 torr, about 54 torr, about 53 torr, about 52 torr, about 51 torr, about 50 torr, about 49 torr, about 48 torr, about 47 torr, about 46 torr, about 45 torr, about 44 torr, about 43 torr, about 42 torr, about 41 torr, about 40 torr, about 39 torr, about 38 torr, about 37 torr, about 36 torr, about 35 torr, about 34 torr, about 33 torr, about 32 torr, about 31 torr, about 30 torr, about 29 torr, about 28 torr, about 27 torr, about 27 torr, about 26 torr, about 25 torr, about 24 torr, about 23 torr, about 22 torr, about 21 torr, about 20 torr, about 19 torr, about 18 torr, about 17 torr, about 16 torr, about 15 torr, about 14 torr, about 13 torr, about 12 torr, about 11 torr, about 10 torr, about 9 torr, about 8 torr, about 7 torr, about 6 torr, about 5 torr, about 4 torr, about 3 torr, about 2 torr, about 1 torr, about 0.9 torr, about 0.8 torr, about 0.7 torr, about 0.6 torr, about 0.5 torr, about 0.4 torr, about 0.3 torr, about 0.2 torr, about 0.1 torr, about 0.001 torr, about 0.0001, and values in between as would be apparent to one of skill.

In some embodiments, the filtrate and wash solvent are placed in a rotary evaporator in order to recover the solvent for reuse in future processing. Herein, a rotary evaporator refers to a device capable of lowering the pressure above a liquid (generally through the use of a vacuum) to, thereby, lower the boiling point of said liquid and thus enable low temperature boiling (i.e., boiling at a temperature lower than the boiling point of the liquid at standard pressure). In such an embodiment, the liquid solvent would be “captured” for reuse after boiling through the use of a condensate-collect flask at the bottom of the condenser (which is generally either a coil passing coolant, or a “cold finger” into which coolant mixtures are placed), capable of catching the distilling solvent after it re-condenses (Harwood and Moody, 1989). In some embodiments, wherein a rotary evaporator is used to recover solvent from the process of the invention, the temperature of the rotary evaporator's water bath is between about 65° C. to about 70° C., including about 65° C., 66° C., 67° C., 68° C., 69° C., 70° C., and values in between, as would be appreciated by one of skill. In some embodiments, wherein a rotary evaporator is used to recover solvent from the process of the invention, the pressure within the rotary evaporator would begin at between about 400 torr to about 500 torr, including about 401 torr, about 402 torr, about 403 torr, about 404 torr, about 405 torr, about 406 torr, about 407 torr, about 408 torr, about 409 torr, about 410 torr, about 411 torr, about 412 torr, about 413 torr, about 414 torr, about 415 torr, about 416 torr, about 417 torr, about 418 torr, about 419 torr, about 420 torr, about 421 torr, about 422 torr, about 423 torr, about 424 torr, about 425 torr, about 426 torr, about 427 torr, about 428 torr, about 429 torr, about 430 torr, about 431 torr, about 432 torr, about 433 torr, about 434 torr, about 435 torr, about 436 torr, about 437 torr, about 438 torr, about 439 torr, about 440 torr, about 441 torr, about 442 torr, about 443 torr, about 444 torr, about 445 torr, about 446 torr, about 447 torr, about 448 torr, about 449 torr, about 450 torr, about 451 torr, about 452 torr, about 453 torr, about 454 torr, about 455 torr, about 456 torr, about 457 torr, about 458 torr, about 459 torr, about 460 torr, about 461 torr, about 462 torr, about 463 torr, about 464 torr, about 465 torr, about 466 torr, about 467 torr, about 468 torr, about 469 torr, about 470 torr, about 471 torr, about 472 torr, about 473 torr, about 474 torr, about 475 torr, about 476 torr, about 477 torr, about 478 torr, about 479 torr, about 480 torr, about 481 torr, about 482 torr, about 483 torr, about 484 torr, about 485 torr, about 486 torr, about 487 torr, about 488 torr, about 489 torr, about 490 torr, about 491 torr, about 492 torr, about 493 torr, about 494 torr, about 495 torr, about 496 torr, about 497 torr, about 498 torr, about 499 torr, about 500 torr, and values in between, as would be immediately apparent to one of skill; and would be reduced to between about 5 to between about 30 torr by the end of recovery, including about 5 torr, about 6 torr, about 7 torr, about 8 torr, about 9 torr, about 10 torr, about 11 torr, about 12 torr, about 13 torr, about 14 about 15 torr, about 16 torr, about 17 torr, about 18 torr, about 19 torr, about 20 torr, about 21 torr, about 22 torr, about 23 torr, about 24 torr, about 25 torr, about 26 torr, about 27 torr, about 28 torr, about 29 torr, about 30 torr, and values in between, as would be apparent to one of skill. Note, however, that the time required to fully remove the solvent will increase from 5 torr to 30 torr (as a larger reduction in pressure leads to quicker evaporation).

In some embodiments, the residue remaining in the evaporator flask will be further processed to yield other valuable constituents from the extract, including terpenes and various neutral cannabinoids. In some embodiments, the dissolved residue is purified prior to solvent recovery by first chilling the combined filtrate and wash solutions at temperatures of between about −45° C. to about 0° C., including about −45° C., about −44° C., about −43° C., about −42° C., about −41° C., about −40° C., about −39° C., about −38° C., about −37° C., about −36° C., about −35° C., about −34° C., about −33° C., about −32° C., about −31° C., about −30° C., about −29° C., about −28° C., about −27° C., about −26° C., about −25° C., about −24° C., about −23° C., about −22° C., about −21° C., about −20° C., about −19° C., about −18° C., about −17° C., about −16° C., about −15° C., about −14° C., about −13° C., about −12° C., about −11° C., about −10° C., about −9° C., about −8° C., about −7° C., about −6° C., about −5° C., about −4° C., about −3° C., about −2° C., about −1° C. about 0° C., and values in between as would be appreciated by those of skill, to allow undesirable substances to precipitate, before immediately filtering the solution. In such embodiments, the filtering is completed through a pad of Celite AKA filtration grade diatomaceous earth or equivalent, as would be apparent to one of skill, to capture any droplets of crud present in the extract.

While cannabis extract produced by any extraction method known to one of skill may be utilized, in some embodiments, an extract produced from an extraction method yielding substantially no THC is used, to minimize the possibility of THC hindering the precipitation/crystallization of THCA. As would be apparent to one of skill, THC and THCA have affinity for one another and, because pure THC is an oil and thus unable to form a solid crystalline structure, its affinity for THCA may affect the ability of THCA to bind and form crystals with other THCA molecules. To compound the issue, as more THCA crystallizes, the THCA:THC ratio trends towards equilibrium, which further inhibits crystallization. Thus, prevention of decarboxylation of THCA to THC can be critical for optimum THCA yields.

In some embodiments, to prevent decarboxylation, plant biomass and extracts produced therefrom are stored at a temperature below which decarboxylation takes place in a significant amount. Because decarboxylation of THC is a factor of both temperature, pressure, and time-decarboxylation begins to occur (visible as small bubbles of CO₂ forming on the solution) when exposed to temperatures in excess of about 70° C. at standard pressure (Bennett, 2016). As such, an extraction method utilized to create the cannabis extract of the invention will, in some embodiments, be one that does not exceed 70° C. at standard pressure.

In some embodiments, the input material used for the disclosed method is a cannabis extract produced using a hydrocarbon solvent at a temperature range sufficient to minimize extraction of unwanted compounds such as waxes, fats, and chlorophylls, which can contribute to a darker, thicker, and relatively less desirable end product. Specific temperature requirements will depend on which hydrocarbon solvent or solvent blend is being used, but appropriate solvent temperature ranges will be known to those skilled in the art.

In some embodiments, the input material used for the disclosed method is a cannabis extract made from “fresh” cannabis, wherein “fresh” refers to cannabis having not been frozen prior to extraction.

In some embodiments, the input material used for the disclosed method is a cannabis extract made from plant material frozen prior to extraction. Generally, in such embodiments, the cannabis plant material is frozen at a temperature of between about −20° C. to about −100° C. In some embodiments, the cannabis plant material is frozen at a temperature of between about −50° C., to about −90° C. In some embodiments, the cannabis plant material is frozen at a temperature of between about −70° C., to about −85° C. In some embodiments, the cannabis plant material is frozen at a temperature of about −20° C., about −21° C., about −22° C., about −23° C., about −24° C., about −25° C., about −26° C., about −27° C., about −28° C., about −29° C., about −30° C., about −31° C., about −32° C., about −33° C., about −34° C., about −35° C., about −36° C., about −37° C., about −38° C., about −39° C., about −40° C., about −41° C., about −42° C., about −43° C., about −44° C., about −45° C., about −46° C., about −47° C., about −48° C., about −49° C., about −50° C., about −51° C., about −52° C., about −53° C., about −54° C., about −55° C., about −56° C., about −57° C., about −58° C., about −59° C., about −60° C., about −61° C., about −62° C., about −63° C., about −64° C., about −65° C., about −66° C., about −67° C., about −68° C., about −69° C., about −70° C., about −71° C., about −72° C., about −73° C., about −74° C., about −75° C., about −76° C., about −77° C., about −78° C., about −79° C., about −80° C., about −81° C., about −82° C., about −83° C., about −84° C., about −85° C., about −86° C., about −87° C., about −88° C., about −89° C., about −90° C., about −91° C., about −92° C., about −93° C., about −94° C., about −95° C., about −96° C., about −97° C., about −98° C., about −99° C., about −100° C., and values in between, or in some embodiments between about −20° C. to about 0° C., and at any such temperatures as would be appreciated by one of skill, for at least about 36 hours, including at least about 40 hours, at least about 44 hours, at least about 48 hours, at least about 56 hours, at least about 64 hours, at least about 72 hours, at least about 96 hours and, in some embodiments, the cannabis plant material may be frozen at the above-referenced temperatures for greater than about 96 hours.

In some embodiments, the input material used for the disclosed method is a cannabis extract made from cannabis sourced from a single seed source, or tissue culture, e.g., with a particular proportion of cannabinoids chosen for a specific end result. In some embodiments, the cannabis is sourced from a single strain of cannabis. In some embodiments, the cannabis is sourced from more than one cannabis strain, including at least 2, at least 3, at least 4, at least 5, at least 6, at least 7, at least 8, at least 9, at least 10, or, in some embodiments, the cannabis strain may be sourced from more than 10 separate cannabis strains.

In some embodiments, the cannabis extract is generated from any form of cannabis plant material, including but not limited to Cannabis sativa, Cannabis indica, Cannabis ruderalis species, as well as all subspecies, varieties, cultivars, and chemovars thereof. As it relates to the invention, “species” also includes any other species of Cannabis, currently known or unknown, in any combination selected by one of skill.

In some embodiments, the extraction step comprises a solvent-based extraction method. In some embodiments, the solvent is supercritical or subcritical carbon dioxide. In some embodiments, the solvent is an alcohol, such as but not limited to, tert-amyl alcohol, benzyl alcohol, 1,4-butanediol, 1,2,4-butanetriol, butanol, 1-butanol, 2-butanol, tert-butyl alcohol, di(propylene glycol) methyl ether, cyclohexanol, cyclopentanol, 1-decanol, 2-2-dimethyl-1-propanol, diethylene glycol, diphenylmethanol, ethanol, ethylene glycol, 2-ethylhexanol, furfuryl alcohol, glycerol, 1-hexanol, 1-heptanol, 1-octanol, 1-nonanol, isobutanol, isopropyl alcohol, methanol, 2-(2-methoxyethoxy)ethanol, 2-methyl-1-butanol, 2-methyl-1-pentanol, 2-methyl-1-propanol, 3-methyl-1-butanol, 3-methyl-2-butanol, 2-methyl-2-propanol, neopentyl alcohol, 1-pentanol, 2-pentanol, phenylmethanol, 1,3-propanediol, 1-propanol, 2-propanol, 2-propen-1-ol, propylene glycol, propylene glycol methyl ether, and triphenylmethanol. In some embodiments, the solvent is a hydrocarbon solvent, such as but not limited to propane, butane, isobutane, pentane, isopentane, petroleum ether, methyl tert-butyl ether, diethyl ether, and mixtures thereof.

In some embodiments, the extraction method utilized is one that produces an extract rich in at least one cannabinoid acid. In some embodiments, the extraction method utilized is one that produces an extract rich in at least one cannabinoid acid, wherein the at least one cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the extraction method utilized is one that produces an extract rich in more than one (e.g. two, three, four, five, six, seven, eight, nine, ten, eleven, twelve, thirteen, fourteen, fifteen, sixteen, seventeen, or more than seventeen) cannabinoid acids. In some embodiments, the extraction method utilized is one that produces an extract rich in more than one cannabinoid acid (e.g., two, three, four, five, six, seven, eight, nine, ten, eleven, twelve, thirteen, fourteen, fifteen, sixteen, seventeen, or more than seventeen), wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the extraction method utilized is one that produces an extract rich in at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA. In some embodiments, the extraction method utilized is one that produces an extract rich in THCA. In some embodiments, the extraction method utilized is one that produces an extract rich in THVCA. In some embodiments, the extraction method utilized is one that produces an extract rich in CBCA. In some embodiments, the extraction method utilized is one that produces an extract rich in CBCVA. In some embodiments, the extraction method utilized is one that produces an extract rich in CBDA. In some embodiments, the extraction method utilized is one that produces an extract rich in CBDVA. In some embodiments, the extraction method utilized is one that produces an extract rich in CBGA. In some embodiments, the extraction method utilized is one that produces an extract rich in CBGVA. In some embodiments, the extraction method utilized is one that produces an extract rich in CBDMA. In some embodiments, the extraction method utilized is one that produces an extract rich in CBDPA. In some embodiments, the extraction method utilized is one that produces an extract rich in CBEA. In some embodiments, the extraction method utilized is one that produces an extract rich in CBFA. In some embodiments, the extraction method utilized is one that produces an extract rich in CBLA. In some embodiments, the extraction method utilized is one that produces an extract rich in CBNA. In some embodiments, the extraction method utilized is one that produces an extract rich in CBNDA. In some embodiments, the extraction method utilized is one that produces an extract rich in CBTA. In some embodiments, the extraction method utilized is one that produces an extract rich in THCPA.

In some embodiments, the extraction method utilized is one that produces an extract rich in more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids. In some embodiments, the extraction method utilized is one that produces an extract rich in more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids, wherein the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

Meaning, in some embodiments, the extraction method utilized is one that produces an extract rich in THCA; in some embodiments, the extraction method utilized is one that produces an extract rich in THCA and THCVA; in some embodiments, the extraction method utilized is one that produces an extract rich in THCA, THCVA and CBCA; in some embodiments, the extraction method utilized is one that produces an extract rich in THCA, THCVA, CBCA, and CBCVA; in some embodiments, the extraction method utilized is one that produces an extract rich in THCA, THCVA, CBCA, CBCVA, and CBDA; in some embodiments, the extraction method utilized is one that produces an extract rich in THCA, THCVA, CBCA, CBCVA, CBDA, and CBDVA; in some embodiments, the extraction method utilized is one that produces an extract rich in THCA, THCVA, CBCA, CBCVA, CBDA, CBDVA, and CBGA; in some embodiments, the extraction method utilized is one that produces an extract rich in THCA, THCVA, CBCA, CBCVA, CBDA, CBDVA, CBGA, and CBGVA; in some embodiments, the extraction method utilized is one that produces an extract rich in THCA, THCVA, CBCA, CBCVA, CBDA, CBDVA, CBGA, CBGVA, and CBDMA; in some embodiments, the extraction method utilized is one that produces an extract rich in THCA, THCVA, CBCA, CBCVA, CBDA, CBDVA, CBGA, CBGVA, CBDMA, and CBDPA; in some embodiments, the extraction method utilized is one that produces an extract rich in THCA, THCVA, CBCA, CBCVA, CBDA, CBDVA, CBGA, CBGVA, CBDMA, CBDPA, and CBEA; in some embodiments, the extraction method utilized is one that produces an extract rich in THCA, THCVA, CBCA, CBCVA, CBDA, CBDVA, CBGA, CBGVA, CBDMA, CBDPA, CBEA, and CBFA; in some embodiments, the extraction method utilized is one that produces an extract rich in THCA, THCVA, CBCA, CBCVA, CBDA, CBDVA, CBGA, CBGVA, CBDMA, CBDPA, CBEA, CBFA, and CBLA; in some embodiments, the extraction method utilized is one that produces an extract rich in THCA, THCVA, CBCA, CBCVA, CBDA, CBDVA, CBGA, CBGVA, CBDMA, CBDPA, CBEA, CBFA, CBLA, and CBNA; in some embodiments, the extraction method utilized is one that produces an extract rich in THCA, THCVA, CBCA, CBCVA, CBDA, CBDVA, CBGA, CBGVA, CBDMA, CBDPA, CBEA, CBFA, CBLA, CBNA, and CBNDA; in some embodiments, the extraction method utilized is one that produces an extract rich in THCA, THCVA, CBCA, CBCVA, CBDA, CBDVA, CBGA, CBGVA, CBDMA, CBDPA, CBEA, CBFA, CBLA, CBNA, CBNDA, and CBTA; in some embodiments, the extraction method utilized is one that produces an extract rich in THCA, THCVA, CBCA, CBCVA, CBDA, CBDVA, CBGA, CBGVA, CBDMA, CBDPA, CBEA, CBFA, CBLA, CBNA, CBNDA, CBTA, and THCPA; or, in some embodiments, the extraction method utilized is one that produces an extract rich in any other two, three, four, five, six, seven, eight, nine, ten, eleven, twelve, thirteen, fourteen, fifteen, sixteen, or seventeen-member combinations one could make from the cannabinoid acids disclosed herein, (i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA) and others known in the art, as would be appreciated by those of skill.

In some embodiments, the extraction method utilized is one that produces an extract possessing substantially no THC or THCA. In some embodiments, the extraction method utilized is one that produces an extract wherein the total THC content (i.e., THCA and THC concentrations), as measured by dry weight, is below about 0.3%, including below about 0.2%, about 0.10%, about 0.01%, about 0.001%, and below about 0.0001%.

In some embodiments, the extraction method utilized is one that produces an extract possessing substantially no CBCA. In some embodiments, the extraction method utilized is one that produces an extract possessing substantially no CBCVA. In some embodiments, the extraction method utilized is one that produces an extract possessing substantially no CBDA. In some embodiments, the extraction method utilized is one that produces an extract possessing substantially no CBDVA. In some embodiments, the extraction method utilized is one that produces an extract possessing substantially no CBGA. In some embodiments, the extraction method utilized is one that produces an extract possessing substantially no CBGVA. In some embodiments, the extraction method utilized is one that produces an extract possessing substantially no CBDMA. In some embodiments, the extraction method utilized is one that produces an extract possessing substantially no CBDPA. In some embodiments, the extraction method utilized is one that produces an extract possessing substantially no CBEA. In some embodiments, the extraction method utilized is one that produces an extract possessing substantially no CBFA. In some embodiments, the extraction method utilized is one that produces an extract possessing substantially no CBLA. In some embodiments, the extraction method utilized is one that produces an extract possessing substantially no CBNA. In some embodiments, the extraction method utilized is one that produces an extract possessing substantially no CBNDA. In some embodiments, the extraction method utilized is one that produces an extract possessing substantially no CBTA. In some embodiments, the extraction method utilized is one that produces an extract possessing substantially no THCPA. In some embodiments, the extraction method utilized is one that produces an extract possessing substantially no THCVA.

In some embodiments, the extraction method utilized is one that produces an extract rich in THCA, and poor in CBCA, CBCVA, CBDA, CBDMA, CBDPA, CBDVA, CBEA, CBFA, CBGA, CBGVA, CBLA, CBNA, CBNDA, CBTA, THCPA, and THCVA. In some embodiments, the extraction method utilized is one that produces an extract rich in THCVA, and poor in CBCA, CBCVA, CBDA, CBDMA, CBDPA, CBDVA, CBEA, CBFA, CBGA, CBGVA, CBLA, CBNA, CBNDA, CBTA, THCA, and THCPA.

In some embodiments, the extraction method utilized is one that produces an extract rich in CBCA, and poor in CBCVA, CBDA, CBDMA, CBDPA, CBDVA, CBEA, CBFA, CBGA, CBGVA, CBLA, CBNA, CBNDA, CBTA, THCA, THCPA, and THCVA. In some embodiments, the extraction method utilized is one that produces an extract rich in CBCVA, and poor in CBCA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the extraction method utilized is one that produces an extract rich in CBDA, and poor in CBCA, CBCVA, CBDMA, CBDPA, CBDVA, CBEA, CBFA, CBGA, CBGVA, CBLA, CBNA, CBNDA, CBTA, THCA, THCPA, and THCVA. In some embodiments, the extraction method utilized is one that produces an extract rich in CBDVA, and poor in CBCA, CBCVA, CBDA, CBDMA, CBDPA, CBEA, CBFA, CBGA, CBGVA, CBLA, CBNA, CBNDA, CBTA, THCA, THCPA, and THCVA.

In some embodiments, the extraction method utilized is one that produces an extract rich in CBGA, and poor in CBCA, CBCVA, CBDA, CBDMA, CBDPA, CBDVA, CBEA, CBFA, CBGVA, CBLA, CBNA, CBNDA, CBTA, THCA, THCPA, and THCVA. In some embodiments, the extraction method utilized is one that produces an extract rich in CBGVA, and poor in CBCA, CBCVA, CBDA, CBDMA, CBDPA, CBDVA, CBEA, CBFA, CBGA, CBLA, CBNA, CBNDA, CBTA, THCA, THCPA, and THCVA.

In some embodiments, the extraction method utilized is one that produces an extract rich in CBDMA, and poor in CBCA, CBCVA, CBDA, CBDPA, CBDVA, CBEA, CBFA, CBGA, CBGVA, CBLA, CBNA, CBNDA, CBTA, THCA, THCPA, and THCVA. In some embodiments, the extraction method utilized is one that produces an extract rich in CBDPA, and poor in CBCA, CBCVA, CBDA, CBDMA, CBDVA, CBEA, CBFA, CBGA, CBGVA, CBLA, CBNA, CBNDA, CBTA, THCA, THCPA, and THCVA.

In some embodiments, the extraction method utilized is one that produces an extract rich in CBEA, and poor in CBCA, CBCVA, CBDA, CBDMA, CBDVA, CBDPA, CBFA, CBGA, CBGVA, CBLA, CBNA, CBNDA, CBTA, THCA, THCPA, and THCVA. In some embodiments, the extraction method utilized is one that produces an extract rich in CBFA, and poor in CBCA, CBCVA, CBDA, CBDMA, CBDVA, CBDPA, CBEA, CBGA, CBGVA, CBLA, CBNA, CBNDA, CBTA, THCA, THCPA, and THCVA.

In some embodiments, the extraction method utilized is one that produces an extract rich in CBLA, and poor in CBCA, CBCVA, CBDA, CBDMA, CBDVA, CBDPA, CBEA, CBGA, CBGVA, CBFA, CBNA, CBNDA, CBTA, THCA, THCPA, and THCVA. In some embodiments, the extraction method utilized is one that produces an extract rich in CBNA, and poor in CBCA, CBCVA, CBDA, CBDMA, CBDVA, CBDPA, CBEA, CBGA, CBGVA, CBFA, CBLA, CBNDA, CBTA, THCA, THCPA, and THCVA.

In some embodiments, the extraction method utilized is one that produces an extract rich in CBNDA, and poor in CBCA, CBCVA, CBDA, CBDMA, CBDVA, CBDPA, CBEA, CBGA, CBGVA, CBFA, CBLA, CBNA, CBTA, THCA, THCPA, and THCVA. In some embodiments, the extraction method utilized is one that produces an extract rich in CBTA, and poor in CBCA, CBCVA, CBDA, CBDMA, CBDVA, CBDPA, CBEA, CBGA, CBGVA, CBFA, CBLA, CBNA, CBNDA, THCA, THCPA, and THCVA.

In some embodiments, the extraction method utilized is one that produces an extract rich in THCPA, and poor in CBCA, CBCVA, CBDA, CBDMA, CBDVA, CBDPA, CBEA, CBGA, CBGVA, CBFA, CBLA, CBNA, CBNDA, THCA, CBTA, and THCVA. In some embodiments, the extraction method utilized is one that produces an extract rich in THCA, and having substantially no CBCA, CBCVA, CBDA, CBDMA, CBDPA, CBDVA, CBEA, CBFA, CBGA, CBGVA, CBLA, CBNA, CBNDA, CBTA, THCPA, or THCVA.

In some embodiments, the extraction method utilized is one that produces an extract rich in THCVA, and having substantially no CBCA, CBCVA, CBDA, CBDMA, CBDPA, CBDVA, CBEA, CBFA, CBGA, CBGVA, CBLA, CBNA, CBNDA, CBTA, THCA, or THCPA. In some embodiments, the extraction method utilized is one that produces an extract rich in CBCA, and having substantially no CBCVA, CBDA, CBDMA, CBDPA, CBDVA, CBEA, CBFA, CBGA, CBGVA, CBLA, CBNA, CBNDA, CBTA, THCA, THCPA, or THCVA.

In some embodiments, the extraction method utilized is one that produces an extract rich in CBCVA, and having substantially no CBCA, CBDA, CBDMA, CBDPA, CBDVA, CBEA, CBFA, CBGA, CBGVA, CBLA, CBNA, CBNDA, CBTA, THCA, THCPA, or THCVA. In some embodiments, the extraction method utilized is one that produces an extract rich in CBDA, and having substantially no CBCA, CBCVA, CBDMA, CBDPA, CBDVA, CBEA, CBFA, CBGA, CBGVA, CBLA, CBNA, CBNDA, CBTA, THCA, THCPA, or THCVA.

In some embodiments, the extraction method utilized is one that produces an extract rich in CBDVA, and having substantially no CBCA, CBCVA, CBDA, CBDMA, CBDPA, CBEA, CBFA, CBGA, CBGVA, CBLA, CBNA, CBNDA, CBTA, THCA, THCPA, or THCVA. In some embodiments, the extraction method utilized is one that produces an extract rich in CBGA, and having substantially no CBCA, CBCVA, CBDA, CBDMA, CBDPA, CBDVA, CBEA, CBFA, CBGVA, CBLA, CBNA, CBNDA, CBTA, THCA, THCPA, or THCVA.

In some embodiments, the extraction method utilized is one that produces an extract rich in CBGVA, and having substantially no CBCA, CBCVA, CBDA, CBDMA, CBDPA, CBDVA, CBEA, CBFA, CBGA, CBLA, CBNA, CBNDA, CBTA, THCA, THCPA, or THCVA. In some embodiments, the extraction method utilized is one that produces an extract rich in CBDMA, and having substantially no CBCA, CBCVA, CBDA, CBDPA, CBDVA, CBEA, CBFA, CBGA, CBGVA, CBLA, CBNA, CBNDA, CBTA, THCA, THCPA, or THCVA.

In some embodiments, the extraction method utilized is one that produces an extract rich in CBDPA, and having substantially no CBCA, CBCVA, CBDA, CBDMA, CBDVA, CBEA, CBFA, CBGA, CBGVA, CBLA, CBNA, CBNDA, CBTA, THCA, THCPA, or THCVA. In some embodiments, the extraction method utilized is one that produces an extract rich in CBEA, and having substantially no CBCA, CBCVA, CBDA, CBDMA, CBDVA, CBDPA, CBFA, CBGA, CBGVA, CBLA, CBNA, CBNDA, CBTA, THCA, THCPA, or THCVA.

In some embodiments, the extraction method utilized is one that produces an extract rich in CBFA, and having substantially no CBCA, CBCVA, CBDA, CBDMA, CBDVA, CBDPA, CBEA, CBGA, CBGVA, CBLA, CBNA, CBNDA, CBTA, THCA, THCPA, or THCVA. In some embodiments, the extraction method utilized is one that produces an extract rich in CBLA, and having substantially no CBCA, CBCVA, CBDA, CBDMA, CBDVA, CBDPA, CBEA, CBGA, CBGVA, CBFA, CBNA, CBNDA, CBTA, THCA, THCPA, or THCVA.

In some embodiments, the extraction method utilized is one that produces an extract rich in CBNA, and having substantially no CBCA, CBCVA, CBDA, CBDMA, CBDVA, CBDPA, CBEA, CBGA, CBGVA, CBFA, CBLA, CBNDA, CBTA, THCA, THCPA, or THCVA. In some embodiments, the extraction method utilized is one that produces an extract rich in CBNDA, and having substantially no CBCA, CBCVA, CBDA, CBDMA, CBDVA, CBDPA, CBEA, CBGA, CBGVA, CBFA, CBLA, CBNA, CBTA, THCA, THCPA, or THCVA.

In some embodiments, the extraction method utilized is one that produces an extract rich in CBTA, and having substantially no CBCA, CBCVA, CBDA, CBDMA, CBDVA, CBDPA, CBEA, CBGA, CBGVA, CBFA, CBLA, CBNA, CBNDA, THCA, THCPA, or THCVA. In some embodiments, the extraction method utilized is one that produces an extract rich in THCPA, and having substantially no CBCA, CBCVA, CBDA, CBDMA, CBDVA, CBDPA, CBEA, CBGA, CBGVA, CBFA, CBLA, CBNA, CBNDA, THCA, CBTA, or THCVA.

In some embodiments, the extraction method utilized is one that produces an extract rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any four of TCBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in all remaining cannabinoid acids (i.e., the remaining cannabinoid acids together comprise less than about 50% of the total composition of the cannabis extract).

In some embodiments, the extraction method utilized is one that produces an extract rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabis extract), and poor in the remaining cannabinoid acid (i.e., the remaining cannabinoid acid comprises less than about 50% of the total composition of the cannabis extract).

In some embodiments, the extraction method utilized is one that produces an extract rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any four of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acids.

In some embodiments, the extraction method utilized is one that produces an extract rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabis extract), and possessing substantially none of the remaining cannabinoid acid.

Methods of testing an extract to determine whether the extract is rich in any one or more specific cannabinoids or cannabinoid acids will be known to those in the art, and may include, in some embodiments, identification and/or quantification by techniques such as comparison to HPLC standards, HPLC, HPLC-MS, GC, GC-MS, IR, MS, 1H-NMR, 13C≡NMR, and/or elemental analysis, as well as other analytical laboratory techniques.

In some embodiments, the cannabinoid acid profile of the cannabis extract is chosen to selectively “affect” various cannabinoid receptors in any of the nervous system, immune system, or other various tissues/organ systems. “Affect,” as it relates to the invention's effects on various cannabinoid receptors, includes psychoactive, therapeutic, prophylactic, and analgesic effects caused by cannabinoid receptor (e.g., CB₁ and/or CB₂ receptor) “agents,” wherein “agent” is broadly defined as a compound that “modulates” (inhibits or activates) the activity of a cannabinoid receptor (e.g., a CB₁ and/or CB₂ receptor).

In some embodiments, the coloration of an extract will be any of yellow, orange, green, brown, or black, including shades between and mixtures thereof; its consistency will be that of a viscous oil, and its appearance will be either translucent or cloudy. In some embodiments, the coloration of an extract will be any of yellow, orange, or brown, including shades between and mixtures thereof; its consistency will be that of a viscous oil, and its appearance will be either translucent or cloudy.

In some embodiments, the input material possesses substantially no plant fats, which is possible when a hydrocarbon solvent (e.g., propane, butane, isobutane, pentane, isopentane, petroleum ether, methyl tert-butyl ether, dimethylether, diethyl ether, and mixtures thereof) or an alcohol solvent (e.g., tert-amyl alcohol, benzyl alcohol, 1,4-butanediol, 1,2,4-butanetriol, butanol, 1-butanol, 2-butanol, tert-butyl alcohol, di(propylene glycol) methyl ether, cyclohexanol, cyclopentanol, 1-decanol, 2-2-dimethyl-1-propanol, diethylene glycol, diphenylmethanol, ethanol, ethylene glycol, 2-ethylhexanol, furfuryl alcohol, glycerol, 1-hexanol, 1-heptanol, 1-octanol, 1-nonanol, isobutanol, isopropyl alcohol, methanol, 2-(2-methoxyethoxy)ethanol, 2-methyl-1-butanol, 2-methyl-1-pentanol, 2-methyl-1-propanol, 3-methyl-1-butanol, 3-methyl-2-butanol, 2-methyl-2-propanol, neopentyl alcohol, 1-pentanol, 2-pentanol, phenylmethanol, 1,3-propanediol, 1-propanol, 2-propanol, 2-propen-1-ol, propylene glycol, propylene glycol methyl ether, and triphenylmethanol) is used in the extraction process at a sufficiently low temperature, a non-limiting example of which includes butane at between about −90° C. to about −45° C., including about −90° C., about −89° C., about −88° C., about −87° C., about −86° C., about −85° C., about −84° C., about −83° C., about −82° C., about −81° C., about −80° C., about −79° C., about −78° C., about −77° C., about −76° C., about −75° C., about −74° C., about −73° C., about −72° C., about −71° C., about −70° C., about −69° C., about −68° C., about −67° C., about −66° C., about −65° C., about −64° C., about −63° C., about −62° C., about −61° C., about −60° C., about −59° C., about −58° C., about −57° C., about −56° C., about −55° C., about −54° C., about −53° C., about −52° C., about −51° C., about −50° C., about −49° C., about −48° C., about −47° C., about −46° C., about −45° C., and values in between as would be appreciated by those of skill.

FIG. 5 illustrates a method of facile purification of cannabinoid acids according to an embodiment of the invention, and is detailed below:

In some embodiments, a method of purification of cannabinoid acids comprises combining a cannabis extract and a polar aprotic solvent to form a solution (501), which may be done by means known to those of skill; mixing the solution to form a cannabinoid acid precipitate (502), which may be done by means known to those of skill, but generally includes an “agitation” step, wherein the solution is agitated by at least one paddle, at least one gate, at least one blade, or another such agitation means known to those of skill; and obtaining the cannabinoid acid precipitate (503), which may be completed via means known to those of skill, with one exemplary embodiment utilizing evaporation through the use of any of, or a combination thereof, a rotary evaporator, a vacuum oven, or ambient evaporation.

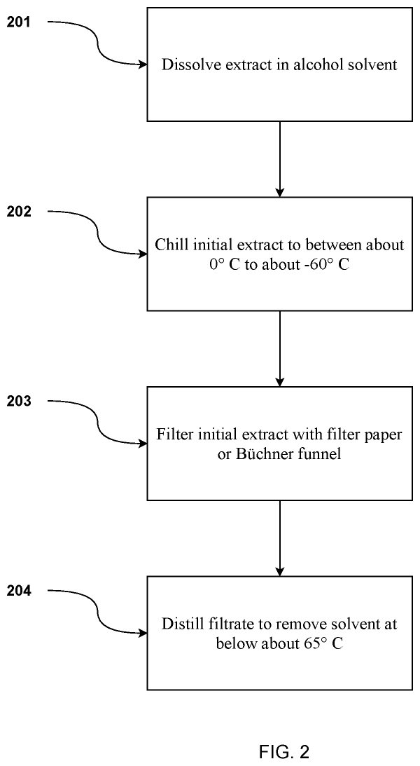

FIG. 2 illustrates a method of removing plant fats and pigments from the initial extract, according to an embodiment of the invention, and is detailed below.

In some embodiments, when plant fats are present in the initial extract, they may be removed prior to use in the method of the invention via, among other means, winterization with an alcohol solvent which, in some embodiments is methanol or 95% alcohol, wherein the extract is dissolved in an alcohol solvent (201), and then chilled to a temperature of between about 0° C. to about −60° C. (202). In some embodiments, the extract is frozen at a temperature of between about −5° C., to about −45° C. In some embodiments, the extract is frozen at a temperature of about 0° C., about −1° C., about −2° C., about −3° C., about −4° C., about −5° C., about −6° C., about −7° C., about −8° C., about −9° C., about −10° C., about −11° C., about −12° C., about −13° C., about −14° C., about −15° C., about −16° C., about −17° C., about −18° C., about −19° C., about −20° C., about −21° C., about −22° C., about −23° C., about −24° C., about −25° C., about −26° C., about −27° C., about −28° C., about −29° C., about −30° C., about −31° C., about −32° C., about −33° C., about −34° C., about −35° C., about −36° C., about −37° C., about −38° C., about −39° C., about −40° C., about −41° C., about −42° C., about −43° C., about −44° C., about −45° C., about −46° C., about −47° C., about −48° C., about −49° C., about −50° C., about −51° C., about −52° C., about −53° C., about −54° C., about −55° C., about −56° C., about −57° C., about −58° C., about −59° C., about −60° C., and values in between, as would be appreciated by one of skill, for between about 30 minutes and about 4 hours, including about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, about 180 minutes, about 185 minutes, about 190 minutes, about 195 minutes, about 200 minutes, about 205 minutes, about 210 minutes, about 215 minutes, about 220 minutes, about 225 minutes, about 230 minutes, about 235 minutes, about 240 minutes, and values in between, as would be apparent to one of skill. Winterization processes and technology therefore may differ, but will be known to those in the art. For example, in some embodiments, purification is performed using the winterization technology known in the art as lipid separation (L-SEP).

After being sufficiently chilled, the initial extract should be filtered prior to recovery of the solvent, said filtration completed using any method known to those of skill. That said, an exemplary embodiment includes pouring the now-chilled initial extract solution quickly through filter paper or Büchner funnel with an 80 to 120 micron frit (203). In some embodiments, the frit is about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, and values in between, as would be apparent to one of skill. In some embodiments, filtration additionally includes diatomaceous earth. Generally, though, any means of filtration capable of filtering out the precipitated lipids is sufficient. After filtration, the solvent is removed from the filtrate via distillation (via any means known to one of skill) at temperatures at or below about 65° C., in order to prevent decarboxylation from occurring (204). Thus, in some embodiments, distillation occurs at temperatures of about 65° C., about 64° C., about 63° C., about 62° C., about 61° C., about 60° C., about 59° C., about 58° C., about 57° C., about 56° C., about 55° C., about 54° C., about 53° C., about 52° C., about 51° C., about 50° C., about 49° C., about 48° C., about 47° C., about 46° C., about 45° C., about 44° C., about 43° C., about 42° C., about 41° C., about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., about 0° C., or values in between, as would be apparent to one of skill.

In some embodiments, wherein lipids and/or plant pigments, including but not limited to chlorophyll, are present in the initial extract, additional solvent washing may be performed during the method of the invention to remove undesirable coloration (e.g., black and green coloration) and increase the purity of the final product. However, additional solvent washing may decrease the overall yield of the final product-so it may instead be recommended the extract be further purified prior to proceeding on with the method of the invention. Said purification may, in some embodiments, be performed by dissolving the extract in a hydrocarbon solvent, non-limiting examples of which include propane, butane, isobutane, pentane, isopentane, petroleum ether, methyl tert-butyl ether, diethyl ether, and mixtures thereof—as would be appreciated by one of skill—and passing the solution through a column packed with adsorbents such as, but not limited to, activated carbons, activated clays, including acid-activated clays; alumina, and silica.

In some embodiments, pigments may also be removed by dissolving the extract in an alcohol solvent, non-limiting examples of which include tert-amyl alcohol, benzyl alcohol, 1,4-butanediol, 1,2,4-butanetriol, butanol, 1-butanol, 2-butanol, tert-butyl alcohol, di(propylene glycol) methyl ether, cyclohexanol, cyclopentanol, 1-decanol, 2-2-dimethyl-1-propanol, diethylene glycol, diphenylmethanol, ethanol, ethylene glycol, 2-ethylhexanol, furfuryl alcohol, glycerol, 1-hexanol, 1-heptanol, 1-octanol, 1-nonanol, isobutanol, isopropyl alcohol, methanol, 2-(2-methoxyethoxy)ethanol, 2-methyl-1-butanol, 2-methyl-1-pentanol, 2-methyl-1-propanol, 3-methyl-1-butanol, 3-methyl-2-butanol, 2-methyl-2-propanol, neopentyl alcohol, 1-pentanol, 2-pentanol, phenylmethanol, 1,3-propanediol, 1-propanol, 2-propanol, 2-propen-1-ol, propylene glycol, propylene glycol methyl ether, and triphenylmethanol; and stirring with an adsorbent, a non-limiting example of which is activated carbon.

In some embodiments, the input material used for the disclosed method is a cannabis extract of lower initial purity. In some embodiments, the input material used for the disclosed method possesses “impurities.” Herein, an “impurity” is any substance that hinders the precipitation of cannabinoid acids, non-limiting examples of which include plant pigments, fats, waxes, and other untargeted extraction compounds, (e.g., compounds other than cannabinoid acids, and in some embodiments, other than one or more of neutral cannabinoids, terpenoids, flavonoids, ketones, aldehydes, esters, and/or organosulfur compounds), as would be apparent to those of skill.

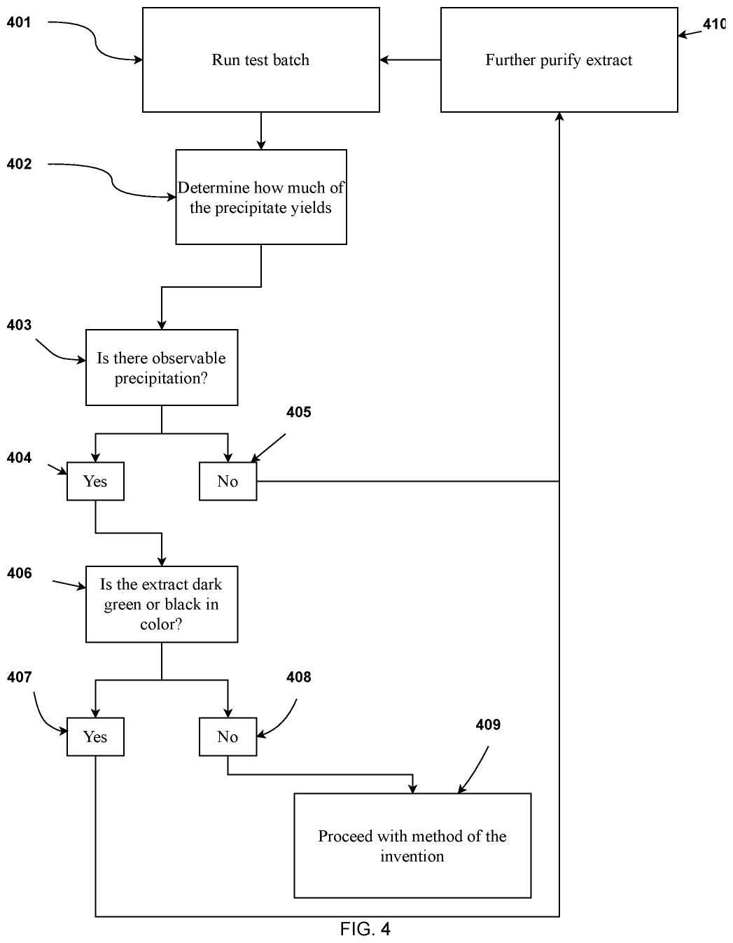

FIG. 4 is a visual representation illustrating a method of determining whether or not initial purification of the cannabis extract is required. It is explained in more depth below.

In some embodiments, one or more initial purification methods may be utilized to purify the extract prior to its use in the process of the invention. In some embodiments, to determine whether the extract needs further purification, a small “test” batch is completed (401) to determine if the extract will yield cannabinoid acids and, if so, how much (402) and at what purity. Various metrics that one of skill would assess when determining if observable precipitation is occurring (403), which determines whether or not additional solvent washing should be performed, include, without limitation, assessing the consistency of the cannabinoid acids in the oil (if the precipitate is “sugary”), which indicates it is precipitating and thus it should yield well (404); if there is substantially no precipitation (405), the yield will likely be low, and additional purification can be performed (410). Also, the color may be assessed. If green or dark green color is present (408), a substantial amount of chlorophyll is likely within an extract, which will yield less desirable, e.g., green, crystals if it is not purified beforehand; thus, additional purification can be performed prior to proceeding with a method of the invention (410). If the precipitate is substantially light (yellow/orange) in color, additional purification need not be performed, and one may proceed with a method of the invention (407). Such metrics will be appreciated by one of skill, and understood based on the general knowledge in the art.

In some embodiments, an extract of lower purity may go through a first purification process prior to being used in the process of the invention, the first purification process comprising dissolving the extract in a hydrocarbon solvent, non-limiting examples of which include propane, butane, isobutane, pentane, isopentane, petroleum ether, and dimethyl ether, and chilling the extract to a temperature and time (which depend on the solvent chosen, as would be apparent to one of skill) sufficient to precipitate unwanted compounds such as waxes, fats, and chlorophylls from the extract and, while maintaining the necessary temperature, the clarified solution (yielded from chilling the extract) is transferred to another vessel before removal of the hydrocarbon solvent-thus leaving behind the undesired retentate materials in the first vessel.

As would be appreciated by those of skill, the color and consistency of the extract can be a sufficient metric as to whether or not additional purification is required. If the extract contains too many impurities, cannabinoid acids, such as but not limited to THCA, will not form small crystals in the initial oil, which greatly lessens the overall efficacy of the crystallization process. Likewise, if the initial extract is black or green, instead of yellow, orange or brown, additional purification can be performed to facilitate optimal crystallization.

In some embodiments, an extract of lower initial purity is at a purity of between about 1%, to about 60%. In some embodiments, an extract of lower initial purity is at a purity of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, and values in between, as would be immediately apparent to one of skill, wherein the purity of the extract (as measured by HPLC, as disclosed herein) is determined by the total cannabinoid acid concentration of the initial extract.

In some embodiments, the input material used for the disclosed method is an extract of lower initial purity. In some embodiments, the input material may go through an initial purification process prior to being used in the above disclosed process, the initial purification process including purification via HPLC with use of a column comprising an adsorbent that includes, but is not limited to, any of acid-activated bleaching clay, silica gel, alumina, activated carbon, and bentonite. As mentioned previously, HPLC can be completed via means known to those of skill; however, as an exemplary embodiment, the system may operate at a pressure of between about 5,000-35,000 kPa, including about 5,000 kPa, about 5,500 kPa, about 6,000 kPa, about 6,500 kPa, about 7,000 kPa, about 7,500 kPa, about 8,000 kPa, about 8,500 kPa, about 9,000 kPa, about 9,500 kPa, about 10,000 kPa, about 10,500 kPa, about 11,000 kPa, about 11,500 kPa, about 12,000 kPa, about 12,500 kPa, about 13,000 kPa, about 13,500 kPa, about 14,000 kPa, about 14,500 kPa, about 15,000 kPa, about 15,500 kPa, about 16,000 kPa, about 16,500 kPa, about 17,000 kPa, about 17,500 kPa, about 18,000 kPa, about 18,500 kPa, about 19,000 kPa, about 19,500 kPa, about 20,000 kPa, about 20,500 kPa, about 21,000 kPa, about 21,500 kPa, about 22,000 kPa, about 22,500 kPa, about 23,000 kPa, about 23,500 kPa, about 24,000 kPa, about 24,500 kPa, about 25,000 kPa, about 25,500 kPa, about 26,000 kPa, about 26,500 kPa, about 27,000 kPa, about 27,500 kPa, about 28,000 kPa, about 28,500 kPa, about 29,000 kPa, about 29,500 kPa, about 30,000 kPa, about 30,500 kPa, about 31,000 kPa, about 31,500 kPa, about 32,000 kPa, about 32,500 kPa, about 33,000 kPa, about 33,500 kPa, about 34,000 kPa, about 34,500 kPa, about 35,000 and values in between, as would be apparent to one of skill; while having a column of between about 2.1 mm and about 4.6 mm in diameter, including about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, and values in between as would be apparent to one of skill; and about 30 mm to about 250 mm in length, including about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, about 175 mm, about 180 mm, about 185 mm, about 190 mm, about 195 mm, about 200 mm, about 205 mm, about 210 mm, about 215 mm, about 220 mm, about 225 mm, about 230 mm, about 235 mm, about 240 mm, about 245 mm, about 250 mm, and values in between, as would be apparent to one of skill (Develosil, 2019; Gerber et al., 2004).

As mentioned, while the method of the invention is viable with extracts of a lower initial purity, in preferred embodiments, the extract has an initial purity of at least 60%, including about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, about 100%, and values in between, as apparent to one of skill, wherein the purity of the extract (as measured by HPLC, as disclosed herein) is determined by the total cannabinoid acid concentration of the initial extract.

In some embodiments, a cannabis extract which is rich in cannabinoid acids (THCA, THCVA, CBCA, CBCVA, CBDA, CBDVA, CBGA, CBGVA, CBDMA, CBDPA, CBEA, CBFA, CBLA, CBNA, CBNDA, CBTA, THCPA, etc.) is added a polar aprotic solvent, in some embodiments preferably an organic nitrile solvent (such as, but not limited to, acetonitrile, acrylonitrile, benzonitrile, butyronitrile, decanenitrile, pivalonitrile, propionitrile, butyronitrile, isobutyronitrile, malononitrile, and succinonitrile), which does not dissolve cannabinoid acids to a significant degree but which readily dissolves undesirable compounds in the extract including terpenes and chlorophylls.

In some embodiments, a “polar aprotic solvent” refers to a solvent that lacks an acidic proton, hydroxyl, and amine groups, and is polar. Polar aprotic solvents are generally known for their ability to dissolve salts. Non-limiting examples of polar aprotic solvents include acetone, acetonitrile, butyronitrile, dichloromethane, dimethylacetamide, dimethylformamide, dimethylpropyleneurea, dimethylsulfoxide, ethyl acetate, hexamethylphosphoric triamide, isobutyronitrile, malononitrile, nitromethane, propionitrile, propylene carbonate, succinonitrile, and tetrahydrofuran. These exemplary polar aprotic solvents have the characteristics as have been reported in the art and therefore known to those of skill below (however, one will appreciate that such characteristics will be readily measurable and may be reported by others in the art or reported in other sources differently; accurate measurements of such characteristics will be those as commonly agreed to by practitioners in the field):

| | | | | | | Dipole |
|---|---|---|---|---|---|---|
| | Chemical | Boiling | | Dielectric | Density | moment |
| Solvent | formula | point | | constant | (g/cm³) | (D) |
| acetone | C₃H₆O | 56.05° | C. | 21.83 | 0.7845 | 2.91 |
| acetonitrile | CH₃CN | 81.3-82.1° | C. | 37.5 | 0.776 | 3.20 |
| dichloromethane | CH₂Cl₂ | 39.6° | C. | 9.08 | 1.3266 | 1.6 |
| dimethylacetamide | CH₃C(O)N(CH₃)₂ | 165.1° | C. | 37.78 | 0.937 | 3.72 |
| dimethylformamide | (CH₃)₂NCHO | 153° | C. | 36.7 | 0.95 | 3.86 |
| dimethylpropyleneurea | (CH₃)₂C₄H₆N₂O | 246.5° | C. | 36.12 | 1.064 | 4.23 |
| dimethylsulfoxide | (CH₃)₂SO | 189° | C. | 46.68 | 1.1 | 3.96 |
| ethyl acetate | C₄H₈O₂ | 77.11° | C. | 6.02 | 0.902 | 1.88 |
| hexamethylphosphoric | [(CH₃)₂N]₃PO | 232.5° | C. | 29.6 | 1.03 | 5.38 |
| triamide | | | | | | |
| tetrahydrofuran | C₄H₈O | 66° | C. | 7.58 | 0.887 | 1.75 |

In some embodiments, the polar aprotic solvents will have a boiling point greater than 66° C. and less than 153° C., or a boiling point between 70° C. and 140° C., between 70° C. and 130° C., between 70° C. and 120° C., between 70° C. and 110° C., between 70° C. and 100° C., between 70° C. and 90° C., or between 70° C. and 85° C. In some embodiments, the polar aprotic solvents will have a boiling point between 75° C. and 140° C., between 75° C. and 130° C., between 75° C. and 120° C., between 75° C. and 110° C., between 75° C. and 100° C., between 75° C. and 90° C., or between 75° C. and 85° C. In some embodiments, the polar aprotic solvents will have a boiling point between 80° C. and 140° C., between 80° C. and 130° C., between 80° C. and 120° C., between 80° C. and 110° C., between 80° C. and 100° C., between 80° C. and 90° C., or between 80° C. and 85° C. In some embodiments, the polar aprotic solvent will have a boiling point of 81.3-82.1° C.

In some embodiments, the polar aprotic solvents will have a dielectric constant greater than 37.5, such as greater than 37.6, greater than 37.7, greater than 37.8, greater than 37.9, greater than 38.0, greater than 38.1, greater than 38.2, or greater than 38.3.

In some embodiments, the polar aprotic solvents will have a density of less than 0.88 g/cm³, less than 0.87 g/cm³, less than 0.86 g/cm³, less than 0.85 g/cm³, less than 0.84 g/cm³, less than 0.83 g/cm³, less than 0.82 g/cm³, less than 0.81 g/cm³, less than 0.80 g/cm³, less than 0.79 g/cm³, or less than 0.78 g/cm³. In some embodiments, the polar aprotic solvent will have a density of 0.776 g/cm³.

In some embodiments, the polar aprotic solvents will have a dipole moment of between 1.80 and 3.70 D, between 1.90 and 3.60 D, between 2.00 and 3.50 D, between 2.10 and 3.40 D, between 2.20 and 3.30 D, or between 2.30 and 3.20 D. In some embodiments, the polar aprotic solvent will have a dipole moment of 3.20 D.

In some embodiments, the polar aprotic solvents will not include any of dimethylacetamide, dimethylformamide, or tetrahydrofuran, or any mixtures thereof.

In some embodiments, “organic nitrile aprotic solvents,” or “nitrile aprotic solvents” will refer to solvents wherein the chemical structure includes a —C≡N functional group and no N—H or O—H bonds, and which have a dielectric constant greater than about 15. Acetonitrile, an organic nitrile solvent useful in the invention, is the simplest organic nitrile, and is produced mainly as a byproduct of acrylonitrile manufacturing. Functionally, acetonitrile is used as a medium-polarity solvent that is miscible with water and a host of other organic solvents, but not saturated hydrocarbons, wherein “saturated” refers to single-bonded carbon atoms (i.e., hydrocarbons having only single bonds between carbon atoms) having hydrogen bonded to carbon's remaining valence electrons. Acetonitrile has a dielectric constant of 38.8, a dipole moment of 3.92, and is useful as a mobile phase in HPLC, and Liquid Chromatography-Mass Spectrometry (LC-MS).

Using one or more of the above embodiments, those skilled in the art can produce a cannabinoid acid precipitate of greater than about 99% purity, including at least about 99.1% purity, at least about 99.2% purity, at least about 99.3% purity, at least about 99.4% purity, at least about 99.5% purity, at least about 99.6% purity, at least about 99.7% purity, at least about 99.9% purity, or at least about 99.99% purity, with an overall yield based on the initial extract of greater than about 75%, including greater than about 76%, greater than about 77%, greater than about 78%, greater than about 79%, greater than about 80% greater than about 81%, greater than about 82%, greater than about 83%, greater than about 84%, greater than about 85%, greater than about 86%, greater than about 87%, greater than about 88%, greater than about 89%, greater than about 90%, greater than about 91%, greater than about 92%, greater than about 93%, greater than about 94%, or greater than about 95%, as measured by HPLC, as disclosed herein, and would be evident to one of skill.

In some embodiments, those skilled in the art can produce a cannabinoid acid precipitate of greater than about 90% purity, including at least about 91.0% purity, at least about 92.0% purity, at least about 93.0% purity, at least about 94.0% purity, at least about 95.0% purity, at least about 95.5% purity, at least about 96.0% purity, at least about 96.5% purity, at least about 97.0% purity, at least about 97.5% purity, at least about 98.0% purity, at least about 98.5% purity, at least about 99.0% purity, or at least about 99.5% purity, as measured by HPLC, as disclosed herein, and would be evident to one of skill.

In some embodiments are methods of removing pigments from the “mother liquor,” and especially dark pigments; the mother liquor, as would be evident to one of skill, is the liquid that remains after the cannabinoid acids have precipitated—the method comprising: cooling the mother liquor to between about −10° C. to about −50° C., including about −10° C., about −11° C., about −12° C., about −13° C., about −14° C., about −15° C., about −16° C., about −17° C., about −18° C., about −19° C., about −20° C., about −21° C., about −22° C., about −23° C., about −24° C., about −25° C., about −26° C., about −27° C., about −28° C., about −29° C., about −30° C., about −31° C., about −32° C., about −33° C., about −34° C., about −35° C., about −36° C., about −37° C., about −38° C., about −39° C., about −40° C., about −41° C., about −42° C., about −43° C., about −44° C., about −45° C., about −46° C., about −47° C., about −48° C., about −49° C., about −50° C., and values in between, as would be apparent to one of skill, for a sufficient time as to allow the pigments to separate, and fall to the bottom of the liquid column or other container, and then the liquid (now as a lightened solution) is quickly poured out and may be filtered. In some embodiments, the mother liquor is cooled to between about −20° C. to about −50° C. As it relates to this specific embodiment of the invention, “filtration” refers to running the liquid over an absorbent filtration media, or rapidly pouring the liquid out, while the bottom contents are still sufficiently immobilized, e.g., before they warm sufficiently to regain viscosity or otherwise return to solution. In either such case, the pigments should be sufficiently settled at the bottom of the liquid column prior to pouring out the lightened solution or filtration. In some embodiments, the lightened solution is poured through an absorbent material so that any dislodged thick crud droplets are effectively stopped in the filter, or filtered through an absorbent material (e.g., diatomaceous earth or a pad of paper towels) that will catch any crud droplets that dislodge from the bottom of the chilled vessel.

The disclosed process also provides an effective means to remediate extracts which are contaminated with pesticides. In the context of the invention, pesticide contaminants may be washed from the crystalline product using the same process of the invention as previously disclosed. If pesticides are present in the initial cannabis extract, after washing with a solvent, as previously disclosed, all trace pesticides will instead be in the solvent/residue. Thus, in some embodiments, after undergoing the process of the invention, pesticide contaminants are substantially undetectable in analytical testing of the cannabinoid acid product material.

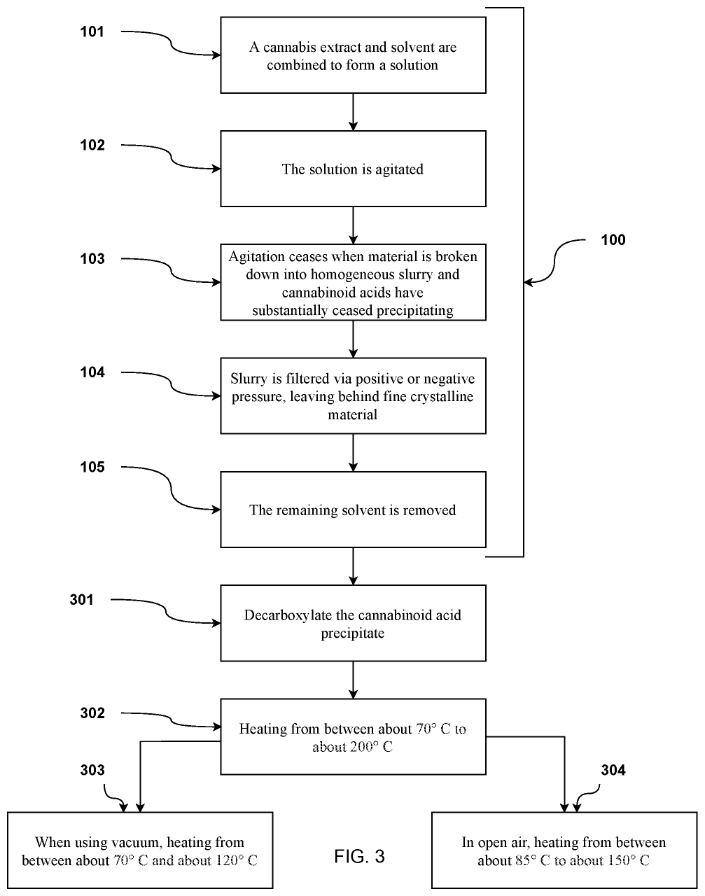

FIG. 3 illustrates a method of facile purification of neutral cannabinoids according to an embodiment of the invention, and is expanded upon below.

In some embodiments, the disclosed process may also be utilized as a means to produce neutral cannabinoids in high purity by partial or complete decarboxylation of the cannabinoid acid precipitate. As mentioned, decarboxylation is a product of temperature, pressure, and time. At standard pressure, decarboxylation begins to occur after material (e.g., a cannabinoid acid precipitate) is exposed to temperatures of at least about 70° C. That said, the time required for decarboxylation to occur decreases with increases in temperature. Meaning, decarboxylation will occur much more rapidly at temperatures in excess of about 200° C., than it will at about 70° C. It is possible to decarboxylate at low temperatures without the assistance of a vacuum (e.g., 85° C.), however, the rate at which decarboxylation would occur would be significantly slower.

Thus, in some embodiments, the cannabinoid acid precipitate is decarboxylated by heating to a temperature of between about 70° C. to about 200° C., until carbon dioxide evolution ceases which, as would be apparent to one of skill, may be from between about 30 minutes, to about 4 hours, depending on the temperature and pressure at which decarboxylation occurs. Through such embodiments, the cannabinoid acid precipitate is decarboxylated to produce a “neutral cannabinoid product” or a “decarboxylated neutral precipitate” or a “decarboxylated precipitate” or a “decarboxylated cannabinoid product” or a “decarboxylated cannabinoid precipitate” or a “decarboxylated precipitate,” or such other terms as will be appreciated in reference to the embodiments and teachings herein. In some embodiments, the cannabinoid acid precipitate is heated to a temperature of about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., about 180° C., about 181° C., about 182° C., about 183° C., about 184° C., about 185° C., about 186° C., about 187° C., about 188° C., about 189° C., about 190° C., about 191° C., about 192° C., about 193° C., about 194° C., about 195° C., about 196° C., about 197° C., about 198° C., about 199° C., about 200° C. and values in between, as would be apparent to one of skill.

Similarly, in some embodiments, to produce a neutral cannabinoid product, the cannabinoid acid precipitate is placed under vacuum or inert gas and heated to a temperature of between about 70° C. and about 120° C. (303), including about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C. and values in between, as apparent to one of skill.

Likewise, in some embodiments, to produce a neutral cannabinoid product, the cannabinoid acid precipitate is heated in open-air conditions (e.g., under ambient pressure) to temperatures of between about 85° C. to about 150° C. (304), including about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., about 148° C., about 149° C., about 150° C., and values in between, as would be apparent to one of skill.

When neutral cannabinoids, e.g., neutral cannabinoid products, are desired, the described methods may be practiced when decarboxylation is completed in vacuum, nitrogen gas blanket, or open-air conditions. In some embodiments, vacuum-assisted or nitrogen gas blanket decarboxylation may be advantageous due to oxygen causing unnecessary and unwanted decomposition when decarboxylation is completed in open-air environments.

In some embodiments, the method of facile purification of the invention produces a cannabinoid acid precipitate rich in at least one cannabinoid. As above (regarding a cannabid extract), a cannabinoid acid precipitate “rich” in one or more cannabinoids will possess the one or more cannabinoids in a concentration that, either alone or in combination, comprises at least about 50% of the total composition of the cannabinoid acid precipitate, including at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or about 100% of the total composition of the cannabinoid acid precipitate.

In some embodiments, a cannabinoid acid precipitate may have a cannabinoid content characterized as “poor,” wherein the cannabinoid content, i.e. the sum of all cannabinoid acid concentrations, comprises less than 50% of the total composition of the cannabinoid acid precipitate. Necessarily, then, in embodiments wherein an extract characterized as “poor” is chosen only containing a single cannabinoid acid, that cannabinoid acid will also comprise less than 50% of the total composition of the cannabinoid acid precipitate.

In some embodiments, the method of facile purification of the invention produces a cannabinoid acid precipitate rich in at least one cannabinoid acid, wherein the at least one cannabinoid acid is any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the method of facile purification of the invention produces a cannabinoid acid precipitate rich in more than one (e.g. two, three, four, five, six, seven, eight, nine, ten, eleven, twelve, thirteen, fourteen, fifteen, sixteen, seventeen, or more than seventeen) cannabinoid acids. In some embodiments, the method of facile purification of the invention produces a cannabinoid acid precipitate rich in more than one cannabinoid acid (e.g., two, three, four, five, six, seven, eight, nine, ten, eleven, twelve, thirteen, fourteen, fifteen, sixteen, seventeen, or more than seventeen), wherein each of the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the method of facile purification of the invention produces a cannabinoid acid precipitate rich in at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA. In some embodiments, the method of facile purification of the invention produces a cannabinoid acid precipitate rich in THCA. In some embodiments, the method of facile purification of the invention produces a cannabinoid acid precipitate rich in THVCA. In some embodiments, the method of facile purification of the invention produces a cannabinoid acid precipitate rich in CBCA. In some embodiments, the method of facile purification of the invention produces a cannabinoid acid precipitate rich in CBCVA. In some embodiments, the method of facile purification of the invention produces a cannabinoid acid precipitate rich in CBDA. In some embodiments, the method of facile purification of the invention produces a cannabinoid acid precipitate rich in CBDVA. In some embodiments, the method of facile purification of the invention produces a cannabinoid acid precipitate rich in CBGA. In some embodiments, the method of facile purification of the invention produces a cannabinoid acid precipitate rich in CBGVA. In some embodiments, the method of facile purification of the invention produces a cannabinoid acid precipitate rich in CBDMA. In some embodiments, the method of facile purification of the invention produces a cannabinoid acid precipitate rich in CBDPA. In some embodiments, the method of facile purification of the invention produces a cannabinoid acid precipitate rich in CBEA. In some embodiments, the method of facile purification of the invention produces a cannabinoid acid precipitate rich in CBFA. In some embodiments, the method of facile purification of the invention produces a cannabinoid acid precipitate rich in CBLA. In some embodiments, the method of facile purification of the invention produces a cannabinoid acid precipitate rich in CBNA. In some embodiments, the method of facile purification of the invention produces a cannabinoid acid precipitate rich in CBNDA. In some embodiments, the method of facile purification of the invention produces a cannabinoid acid precipitate rich in CBTA. In some embodiments, the method of facile purification of the invention produces a cannabinoid acid precipitate rich in THCPA.

In some embodiments, the method of facile purification of the invention produces a cannabinoid acid precipitate rich in more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids. In some embodiments, the method of facile purification of the invention produces a cannabinoid acid precipitate rich in more than one cannabinoid acid, including at least two, at least three, at least four, at least five, at least six, at least seven, at least eight, at least nine, at least ten, at least eleven, at least twelve, at least thirteen, at least fourteen, at least fifteen, at least sixteen, at least seventeen, or more than seventeen cannabinoid acids, wherein the more than one cannabinoid acids are any of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

Meaning, in some embodiments, the method of facile purification of the invention produces a cannabinoid acid precipitate rich in THCA; in some embodiments, the method of facile purification of the invention produces a cannabinoid acid precipitate rich in THCA and THCVA; in some embodiments, the method of facile purification of the invention produces a cannabinoid acid precipitate rich in THCA, THCVA and CBCA; in some embodiments, the method of facile purification of the invention produces a cannabinoid acid precipitate rich in THCA, THCVA, CBCA, and CBCVA; in some embodiments, the method of facile purification of the invention produces a cannabinoid acid precipitate rich in THCA, THCVA, CBCA, CBCVA, and CBDA; in some embodiments, the method of facile purification of the invention produces a cannabinoid acid precipitate rich in THCA, THCVA, CBCA, CBCVA, CBDA, and CBDVA; in some embodiments, the method of facile purification of the invention produces a cannabinoid acid precipitate rich in THCA, THCVA, CBCA, CBCVA, CBDA, CBDVA, and CBGA; in some embodiments, the method of facile purification of the invention produces a cannabinoid acid precipitate rich in THCA, THCVA, CBCA, CBCVA, CBDA, CBDVA, CBGA, and CBGVA; in some embodiments, the method of facile purification of the invention produces a cannabinoid acid precipitate rich in THCA, THCVA, CBCA, CBCVA, CBDA, CBDVA, CBGA, CBGVA, and CBDMA; in some embodiments, the method of facile purification of the invention produces a cannabinoid acid precipitate rich in THCA, THCVA, CBCA, CBCVA, CBDA, CBDVA, CBGA, CBGVA, CBDMA, and CBDPA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, and CBEA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, and CBFA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, CBFA, and CBLA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, CBFA, CBLA, and CBNA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, CBFA, CBLA, CBNA, and CBNDA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, CBFA, CBLA, CBNA, CBNDA, and CBTA; in some embodiments, the method of facile purification of the invention produces a cannabinoid acid precipitate rich in THCA, THCVA, CBCA, CBCVA, CBDA, CBDVA, CBGA, CBGVA, CBDMA, CBDPA, CBEA, CBFA, CBLA, CBNA, CBNDA, CBTA, and THCPA; or, in some embodiments, the method of facile purification of the invention produces a cannabinoid acid precipitate rich in any other two, three, four, five, six, seven, eight, nine, ten, eleven, twelve, thirteen, fourteen, fifteen, sixteen, or seventeen-member combinations one could make from the cannabinoid acids disclosed herein, (i.e., CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA) and others known in the art, as would be appreciated by those of skill.

In some embodiments, the method of facile purification of the invention produces a cannabinoid acid precipitate possessing relatively little THCA and/or THC, or a neutral cannabinoid product possessing relatively little THC. In some embodiments, the method of facile purification of the invention produces a cannabinoid acid precipitate possessing relatively little THCA and/or THC, or a neutral cannabinoid product possessing relatively little THC, by using a cannabis extract produced from industrial hemp, e.g., cannabis plant material wherein the total THC content (i.e., THCA and THC concentrations), as measured by dry weight, is below about 0.3%, including below about 0.2% and below about 0.1%.

In some embodiments, the method of facile purification of the invention produces a cannabinoid acid precipitate possessing substantially no CBCA. In some embodiments, the method of facile purification of the invention produces a cannabinoid acid precipitate possessing substantially no CBCVA. In some embodiments, the method of facile purification of the invention produces a cannabinoid acid precipitate possessing substantially no CBDA. In some embodiments, the method of facile purification of the invention produces a cannabinoid acid precipitate possessing substantially no CBDVA. In some embodiments, the method of facile purification of the invention produces a cannabinoid acid precipitate possessing substantially no CBGA. In some embodiments, the method of facile purification of the invention produces a cannabinoid acid precipitate possessing substantially no CBGVA. In some embodiments, the method of facile purification of the invention produces a cannabinoid acid precipitate possessing substantially no CBDMA. In some embodiments, the method of facile purification of the invention produces a cannabinoid acid precipitate possessing substantially no CBDPA. In some embodiments, the method of facile purification of the invention produces a cannabinoid acid precipitate possessing substantially no CBEA. In some embodiments, the method of facile purification of the invention produces a cannabinoid acid precipitate possessing substantially no CBFA. In some embodiments, the method of facile purification of the invention produces a cannabinoid acid precipitate possessing substantially no CBLA. In some embodiments, the method of facile purification of the invention produces a cannabinoid acid precipitate possessing substantially no CBNA. In some embodiments, the method of facile purification of the invention produces a cannabinoid acid precipitate possessing substantially no CBNDA. In some embodiments, the method of facile purification of the invention produces a cannabinoid acid precipitate possessing substantially no CBTA. In some embodiments, the method of facile purification of the invention produces a cannabinoid acid precipitate possessing substantially no THCPA. In some embodiments, the method of facile purification of the invention produces a cannabinoid acid precipitate possessing substantially no THCVA.

In some embodiments, the method of facile purification of the invention produces a cannabinoid acid precipitate rich in THCA, and poor in CBCA, CBCVA, CBDA, CBDMA, CBDPA, CBDVA, CBEA, CBFA, CBGA, CBGVA, CBLA, CBNA, CBNDA, CBTA, THCPA, and THCVA. In some embodiments, the method of facile purification of the invention produces a cannabinoid acid precipitate rich in THCVA, and poor in CBCA, CBCVA, CBDA, CBDMA, CBDPA, CBDVA, CBEA, CBFA, CBGA, CBGVA, CBLA, CBNA, CBNDA, CBTA, THCA, and THCPA.

In some embodiments, the method of facile purification of the invention produces a cannabinoid acid precipitate rich in CBCA, and poor in CBCVA, CBDA, CBDMA, CBDPA, CBDVA, CBEA, CBFA, CBGA, CBGVA, CBLA, CBNA, CBNDA, CBTA, THCA, THCPA, and THCVA. In some embodiments, the method of facile purification of the invention produces a cannabinoid acid precipitate rich in CBCVA, and poor in CBCA, CBDA, CBDMA, CBDPA, CBDVA, CBEA, CBFA, CBGA, CBGVA, CBLA, CBNA, CBNDA, CBTA, THCA, THCPA, and THCVA.

In some embodiments, the method of facile purification of the invention produces a cannabinoid acid precipitate rich in CBDA, and poor in CBCA, CBCVA, CBDMA, CBDPA, CBDVA, CBEA, CBFA, CBGA, CBGVA, CBLA, CBNA, CBNDA, CBTA, THCA, THCPA, and THCVA. In some embodiments, the method of facile purification of the invention produces a cannabinoid acid precipitate rich in CBDVA, and poor in CBCA, CBCVA, CBDA, CBDMA, CBDPA, CBEA, CBFA, CBGA, CBGVA, CBLA, CBNA, CBNDA, CBTA, THCA, THCPA, and THCVA.

In some embodiments, the method of facile purification of the invention produces a cannabinoid acid precipitate rich in CBGA, and poor in CBCA, CBCVA, CBDA, CBDMA, CBDPA, CBDVA, CBEA, CBFA, CBGVA, CBLA, CBNA, CBNDA, CBTA, THCA, THCPA, and THCVA. In some embodiments, the method of facile purification of the invention produces a cannabinoid acid precipitate rich in CBGVA, and poor in CBCA, CBCVA, CBDA, CBDMA, CBDPA, CBDVA, CBEA, CBFA, CBGA, CBLA, CBNA, CBNDA, CBTA, THCA, THCPA, and THCVA.

In some embodiments, the method of facile purification of the invention produces a cannabinoid acid precipitate rich in CBDMA, and poor in CBCA, CBCVA, CBDA, CBDPA, CBDVA, CBEA, CBFA, CBGA, CBGVA, CBLA, CBNA, CBNDA, CBTA, THCA, THCPA, and THCVA. In some embodiments, the method of facile purification of the invention produces a cannabinoid acid precipitate rich in CBDPA, and poor in CBCA, CBCVA, CBDA, CBDMA, CBDVA, CBEA, CBFA, CBGA, CBGVA, CBLA, CBNA, CBNDA, CBTA, THCA, THCPA, and THCVA.

In some embodiments, the method of facile purification of the invention produces a cannabinoid acid precipitate rich in CBEA, and poor in CBCA, CBCVA, CBDA, CBDMA, CBDVA, CBDPA, CBFA, CBGA, CBGVA, CBLA, CBNA, CBNDA, CBTA, THCA, THCPA, and THCVA. In some embodiments, the method of facile purification of the invention produces a cannabinoid acid precipitate rich in CBFA, and poor in CBCA, CBCVA, CBDA, CBDMA, CBDVA, CBDPA, CBEA, CBGA, CBGVA, CBLA, CBNA, CBNDA, CBTA, THCA, THCPA, and THCVA.

In some embodiments, the method of facile purification of the invention produces a cannabinoid acid precipitate rich in CBLA, and poor in CBCA, CBCVA, CBDA, CBDMA, CBDVA, CBDPA, CBEA, CBGA, CBGVA, CBFA, CBNA, CBNDA, CBTA, THCA, THCPA, and THCVA. In some embodiments, the method of facile purification of the invention produces a cannabinoid acid precipitate rich in CBNA, and poor in CBCA, CBCVA, CBDA, CBDMA, CBDVA, CBDPA, CBEA, CBGA, CBGVA, CBFA, CBLA, CBNDA, CBTA, THCA, THCPA, and THCVA.

In some embodiments, the method of facile purification of the invention produces a cannabinoid acid precipitate rich in CBNDA, and poor in CBCA, CBCVA, CBDA, CBDMA, CBDVA, CBDPA, CBEA, CBGA, CBGVA, CBFA, CBLA, CBNA, CBTA, THCA, THCPA, and THCVA. In some embodiments, the method of facile purification of the invention produces a cannabinoid acid precipitate rich in CBTA, and poor in CBCA, CBCVA, CBDA, CBDMA, CBDVA, CBDPA, CBEA, CBGA, CBGVA, CBFA, CBLA, CBNA, CBNDA, THCA, THCPA, and THCVA.

In some embodiments, the method of facile purification of the invention produces a cannabinoid acid precipitate rich in THCPA, and poor in CBCA, CBCVA, CBDA, CBDMA, CBDVA, CBDPA, CBEA, CBGA, CBGVA, CBFA, CBLA, CBNA, CBNDA, THCA, CBTA, and THCVA. In some embodiments, the method of facile purification of the invention produces a cannabinoid acid precipitate rich in THCA, and having substantially no CBCA, CBCVA, CBDA, CBDMA, CBDPA, CBDVA, CBEA, CBFA, CBGA, CBGVA, CBLA, CBNA, CBNDA, CBTA, THCPA, or THCVA.

In some embodiments, the method of facile purification of the invention produces a cannabinoid acid precipitate rich in THCVA, and having substantially no CBCA, CBCVA, CBDA, CBDMA, CBDPA, CBDVA, CBEA, CBFA, CBGA, CBGVA, CBLA, CBNA, CBNDA, CBTA, THCA, or THCPA. In some embodiments, the method of facile purification of the invention produces a cannabinoid acid precipitate rich in CBCA, and having substantially no CBCVA, CBDA, CBDMA, CBDPA, CBDVA, CBEA, CBFA, CBGA, CBGVA, CBLA, CBNA, CBNDA, CBTA, THCA, THCPA, or THCVA.

In some embodiments, the method of facile purification of the invention produces a cannabinoid acid precipitate rich in CBCVA, and having substantially no CBCA, CBDA, CBDMA, CBDPA, CBDVA, CBEA, CBFA, CBGA, CBGVA, CBLA, CBNA, CBNDA, CBTA, THCA, THCPA, or THCVA. In some embodiments, the method of facile purification of the invention produces a cannabinoid acid precipitate rich in CBDA, and having substantially no CBCA, CBCVA, CBDMA, CBDPA, CBDVA, CBEA, CBFA, CBGA, CBGVA, CBLA, CBNA, CBNDA, CBTA, THCA, THCPA, or THCVA.

In some embodiments, the method of facile purification of the invention produces a cannabinoid acid precipitate rich in CBDVA, and having substantially no CBCA, CBCVA, CBDA, CBDMA, CBDPA, CBEA, CBFA, CBGA, CBGVA, CBLA, CBNA, CBNDA, CBTA, THCA, THCPA, or THCVA. In some embodiments, the method of facile purification of the invention produces a cannabinoid acid precipitate rich in CBGA, and having substantially no CBCA, CBCVA, CBDA, CBDMA, CBDPA, CBDVA, CBEA, CBFA, CBGVA, CBLA, CBNA, CBNDA, CBTA, THCA, THCPA, or THCVA.

In some embodiments, the method of facile purification of the invention produces a cannabinoid acid precipitate rich in CBGVA, and having substantially no CBCA, CBCVA, CBDA, CBDMA, CBDPA, CBDVA, CBEA, CBFA, CBGA, CBLA, CBNA, CBNDA, CBTA, THCA, THCPA, or THCVA. In some embodiments, the method of facile purification of the invention produces a cannabinoid acid precipitate rich in CBDMA, and having substantially no CBCA, CBCVA, CBDA, CBDPA, CBDVA, CBEA, CBFA, CBGA, CBGVA, CBLA, CBNA, CBNDA, CBTA, THCA, THCPA, or THCVA.

In some embodiments, the method of facile purification of the invention produces a cannabinoid acid precipitate rich in CBDPA, and having substantially no CBCA, CBCVA, CBDA, CBDMA, CBDVA, CBEA, CBFA, CBGA, CBGVA, CBLA, CBNA, CBNDA, CBTA, THCA, THCPA, or THCVA. In some embodiments, the method of facile purification of the invention produces a cannabinoid acid precipitate rich in CBEA, and having substantially no CBCA, CBCVA, CBDA, CBDMA, CBDVA, CBDPA, CBFA, CBGA, CBGVA, CBLA, CBNA, CBNDA, CBTA, THCA, THCPA, or THCVA.

In some embodiments, the method of facile purification of the invention produces a cannabinoid acid precipitate rich in CBFA, and having substantially no CBCA, CBCVA, CBDA, CBDMA, CBDVA, CBDPA, CBEA, CBGA, CBGVA, CBLA, CBNA, CBNDA, CBTA, THCA, THCPA, or THCVA. In some embodiments, the method of facile purification of the invention produces a cannabinoid acid precipitate rich in CBLA, and having substantially no CBCA, CBCVA, CBDA, CBDMA, CBDVA, CBDPA, CBEA, CBGA, CBGVA, CBFA, CBNA, CBNDA, CBTA, THCA, THCPA, or THCVA.

In some embodiments, the method of facile purification of the invention produces a cannabinoid acid precipitate rich in CBNA, and having substantially no CBCA, CBCVA, CBDA, CBDMA, CBDVA, CBDPA, CBEA, CBGA, CBGVA, CBFA, CBLA, CBNDA, CBTA, THCA, THCPA, or THCVA. In some embodiments, the method of facile purification of the invention produces a cannabinoid acid precipitate rich in CBNDA, and having substantially no CBCA, CBCVA, CBDA, CBDMA, CBDVA, CBDPA, CBEA, CBGA, CBGVA, CBFA, CBLA, CBNA, CBTA, THCA, THCPA, or THCVA.

In some embodiments, the method of facile purification of the invention produces a cannabinoid acid precipitate rich in CBTA, and having substantially no CBCA, CBCVA, CBDA, CBDMA, CBDVA, CBDPA, CBEA, CBGA, CBGVA, CBFA, CBLA, CBNA, CBNDA, THCA, THCPA, or THCVA. In some embodiments, the method of facile purification of the invention produces a cannabinoid acid precipitate rich in THCPA, and having substantially no CBCA, CBCVA, CBDA, CBDMA, CBDVA, CBDPA, CBEA, CBGA, CBGVA, CBFA, CBLA, CBNA, CBNDA, THCA, CBTA, or THCVA.

In some embodiments, the method of facile purification of the invention produces a cannabinoid acid precipitate rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any four of TCBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in all remaining cannabinoid acids (i.e., the remaining cannabinoid acids together comprise less than about 50% of the total composition of the cannabinoid acid precipitate).

In some embodiments, the method of facile purification of the invention produces a cannabinoid acid precipitate rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., together comprising at least about 50% of the total composition of the cannabinoid acid precipitate), and poor in the remaining cannabinoid acid (i.e., the remaining cannabinoid acid comprises less than about 50% of the total composition of the cannabinoid acid precipitate).

In some embodiments, the method of facile purification of the invention produces a cannabinoid acid precipitate rich in one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any two of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any three of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any four of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any five of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any six of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any seven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any eight of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any nine of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any ten of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any eleven of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any twelve of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any thirteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any fourteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any fifteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acids.

In some embodiments, the method of facile purification of the invention produces a cannabinoid acid precipitate rich in any sixteen of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA (i.e., comprising at least 50% of the total composition of the cannabinoid acid precipitate), and possessing substantially none of the remaining cannabinoid acid.

Methods of testing a cannabinoid acid precipitate to determine whether the cannabinoid acid precipitate is rich in any one or more specific cannabinoids or cannabinoid acids will be known to those in the art, and may include, in some embodiments, identification and/or quantification by techniques such as comparison to HPLC standards, HPLC, HPLC-MS, GC, GC-MS, IR, MS, 1H-NMR, 13C≡NMR, and/or elemental analysis, as well as other analytical laboratory techniques.

In some embodiments, the purified extract yielded as a result of the invention possesses a reduced concentration of pigments, such as green, black, and brown pigments associated with impurities including, but not limited to, plant pigments (e.g. chlorophyll), plant fats and other lipids, etc.-thus possessing a more commercially desirable color profile—and an increased concentration of flavor compounds. Herein, “flavor compounds” refer to those which yield a perceptual impression of a substance or chemical by activating one's gustatory and olfactory senses. As it relates to the invention, compounds that impart flavor (i.e., “flavor compounds”) include but are not limited to, terpenoids (the term given to terpenes which have oxidized and gained an additional oxygen-containing functional group), which give plants their distinctive smells and tastes; aldehydes, which are an organic functional group with the structure—C(H)═O, and are known to possess a fruity, green, pungent flavor like that found in coriander; esters also an organic function group, having the structure—O—C(R)═O, and also known to possess fruity flavors like that found in banana; ketones also an organic functional group, having the structure R2C═O, wherein “R” can be a variety of carbon-containing substituents, and are known for having a “fruity” aroma; organosulfur compounds, i.e., organic compounds that contain sulfur, which are a precursor for a variety of “skunky” compounds, such as, but not limited to, 3-methyl-2-butene-1-thiol; flavonoids, a diverse group of phytonutrients found in nearly all fruits and vegetables responsible for their coloration; and anthocyanins, which are water-soluble pigments that, depending on their pH, may appear red, purple, blue, or black (Specter, 2009; Eriksson et al., 2012; Szalay, 2015; Davies, 2004; Archetti, 2011).

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternative embodiments are contemplated, and can be made without departing from the spirit and scope of the invention.

### Example 1: >99% Pure Crystalline THCA Produced on a Kilogram Scale

The starting material was a cannabis extract that tested as 66.54% THCA. This initial cannabis extract starting material was produced from cold butane extraction (−50° C.) of THCA-predominant cannabis biomass (i.e., cannabis plant material).

This cannabis extract starting material was then further purified by an initial purification step comprising purification with a colunn of acid-activated bleaching clay (Eastchem, Activated Bleaching Earth, F2 grade).

Following the initial purification step, 1,000.4 g of the further purified cannabis extract and 400 mL of acetonitrile were added to a 2 liter glass reaction kettle. The kettle was fitted with a lid, mechanical stirrer, and reflux condenser. The kettle was then placed in a water bath maintained at a temperature of 60° C.

Stirring was initiated as soon as the extract was warmed sufficiently to lower its viscosity and allow stirring (ca. 20 minutes). The mixture was stirred at 800 rpm for 30 minutes, causing a large proportion of the extract to dissolve with the remaining undissolved THCA suspended as fine particles. The heated water bath was removed and the solution was allowed to cool for another 30 minutes, causing an increasing amount of THCA to precipitate out of solution. The stirrer and condenser were removed and replaced with stoppers. The reaction kettle was placed overnight in a refrigerator maintained at a temperature of 2 to 5° C.

The next day the majority of the suspension volume in the reaction kettle was replaced by a semi-solid crystalline cake. Sitting on top of this cake was an approximately 1 cm layer of viscous oil and above the oil was approximately 130 mL of acetonitrile solution. The top acetonitrile solution was decanted into a beaker and covered. The thick oil layer was then carefully removed using a spatula. The separated acetonitrile solution was then placed back into the reaction kettle and the crystalline cake was broken up into a thick slurry using a spatula.

The slurry was transferred to a 2 L Büchner funnel with a coarse frit and vacuum filtration was performed until the filtrate ceased collecting.

Next, 380 mL of fresh acetonitrile that had been chilled in a refrigerator (2 to 5° C.) was poured on top of the crystal cake. The cake was agitated with a spatula to break it up and suspend it in solution.

Vacuum filtration was then initiated and the crystalline cake was manually packed to enable the removal of as much filtrate as possible. 360 mL of chilled fresh acetonitrile was then added on top of the packed crystal cake and the vacuum was disconnected from the filtration assembly in order to allow a slow and even gravity filtration of the crystal cake.

After the majority of the filtrate had flowed through and collection had nearly ceased the vacuum was reattached and initiated to collect the remaining solution. The clean white crystalline THCA (cannabinoid acid precipitate) was then transferred to a large Pyrex baking dish and the residual solvent was allowed to evaporate overnight.

The final yield was 660.2 grams of white crystalline THCA precipitate with a purity of 99.23%, delivering an overall yield of 98.4%.

### Example 2: High Purity CBDA, CBGA, and THCA Produced on a Kilogram Scale

The starting material was a cannabis extract that tested 54.73% CBDA, 5.26% CBGA, and 2.81% THCA. The extract was produced from cold methanol extraction (−35° C.) of CBDA predominant cannabis biomass.

This cannabis extract starting material was then further purified by an initial purification step comprising purification with activated carbon in a rotating bed reactor and then removal of the solvent.

Following the initial purification step, 1,000.2 g of the further purified cannabis extract and 500 mL of acetonitrile were added to a 2 liter glass reaction kettle. The extract was broken up with a spatula in order to facilitate stirring. The kettle was then fitted with a lid and a mechanical stirrer with a radial impeller to create increased sheer during stirring.

Stirring was set to 500 rpm and then increased to 1,200 rpm after all chunks of extract were broken down into pieces smaller than 5 mm. The stirrer was left to run overnight (approx. 18 hours total).

The ambient temperature in the laboratory was maintained between 16 to 21° C. during the entire process.

The following day it was observed that the solution had become much thicker as additional CBDA and CBGA had precipitated out of solution. The consistency of the precipitated cannabinoids were fine crystals with no observable chunks of extract remaining.

The slurry was transferred to a 2 L Büchner funnel with a coarse frit and vacuum filtration was performed until the filtrate ceased collecting. Next, 400 mL of acetonitrile was poured on top of the crystal cake. The crystalline material was suspended using a spatula and then the mixture was vacuum filtered with the crystalline cake being manually packed to enable the removal of as much filtrate as possible (such as also described in Example 1).

Another 400 mL of acetonitrile was poured on top of the crystal cake. The crystalline material was again suspended using a spatula and then vacuum filtered with the crystalline cake being manually packed to enable the removal of as much filtrate as possible. 350 mL of acetonitrile was then added on top of the packed crystal cake and the vacuum was disconnected from the filtration assembly in order to allow a slow and even gravity filtration of the crystal cake.

After the majority of the acetonitrile solution had flowed through and collection had nearly ceased the vacuum was reattached and initiated to collect the remaining filtrate. The clean white crystalline cannabinoid acids (cannabinoid acid precipitate) were then transferred to a large Pyrex baking dish and the residual solvent was allowed to evaporate overnight.

The yielded white crystalline material weighed 593.8 g and tested 84.68% CBDA, 8.47% CBGA, and 4.63% THCA for a combined cannabinoid purity of 97.78%, and delivering an overall yield of 92.4%.

### Example 3: >99.9% Pure THCA and THCVA Produced on a 30 Kilogram Scale

The starting material was a cannabis extract that tested 64.53% THCA and 0.31% THCVA. The extract was produced from cryo butane extraction (−75° C.) of THCA-predominant cannabis biomass. No further purification was performed.

To a 50 L jacketed reactor was added 11 liters of acetonitrile. Mechanical stirring at 400 rpm was initiated and the circulating jacket fluid was heated to a maintained temperature of 55° C. 30,007.8 g of the cannabis extract and was then added in portions so as to avoid difficulty with stirring.

After the cannabis extract addition was complete, the stirring rate was increased to 1,000 rpm and the stirring continued for another 30 minutes until all oil was dissolved and only fine crystalline material was visible in the suspension.

Chilling of the jacket fluid was then initiated and the mixture temperature was brought down to 2° C. As the temperature of the mixture decreased an increasing amount of cannabinoid acids precipitated out of solution.

After 4 hours of stirring at 2° C., precipitation of cannabinoid acids had appeared to have substantially ceased, as evidenced by the lack of observable change in the thickness of the slurry for a substantial period of time.

Stirring was reduced to 500 rpm and the slurry was transferred to a 50 L jacketed Nutsche filter. The jacket fluid in the Nutsche filter was maintained at a temperature of 2° C. The mixture was immediately filtered using 1 bar of positive pressure.

Ten liters of fresh acetonitrile that had been stored in a refrigerator at a temperature of 1 to 3° C. were then added to the filter cake. The filter cake was broken up with a manual agitating blade. After all the solids were suspended in solution, the agitating blade was quickly removed and the mixture filtered with positive pressure.

Eight liters of fresh chilled acetonitrile were added to the top of the filter cake and then allowed to slowly gravity filter through. Positive air pressure of 1 bar was initiated once collection of filtrate had slowed. After no more filtrate could be collected the jacket fluid temperature was increased to 35° C. and air flow was continued for 2 hours until the filter cake was deemed to be dried of solvent.

The yielded white crystalline material (cannabinoid acid precipitate) weighed 18,896.6 grams and tested 99.18% THCA and 0.80% THCVA for a combined cannabinoid purity of 99.98%, and delivering an overall yield of 97.1%.

Purification of the filtrate solution: The combined filtrates were transferred to three 20 liter HDPE carboys and then placed into a chest freezer that was maintained at a temperature of −23 to −18° C. After 24 hours in the freezer it was observed that the filtrate solution had become much lighter in color and clearer, starting from an originally opaque brown solution to a bright yellow/orange solution. Dark brown sludge was observed stuck to the bottom of the carboys. A 3 liter Büchner funnel was prepared with an approximately 1 inch bed of acetonitrile wetted Celite on top of the filtration surface. Vacuum was initiated and then each carboy's contents were quickly poured through the funnel so as to not allow time for the brown sludge to warm and lose viscosity. The acetonitrile solvent then was recovered in a rotary evaporator, delivering a purified extract with a more commercially desirable color.

While the methods described and illustrated herein may include particular steps, it should be apparent that other methods including fewer, more, or different steps than those described and shown are also within the spirit and scope of the invention. The methods and uses of any device or apparatus discussed and associated steps shown herein therefore should be understood as being provided for purposes of illustration, not limitation. It should be further understood that the specific order or hierarchy of steps in the methods and uses of a device disclosed are only exemplary approaches. Based upon user and design preferences, the specific order or hierarchy of steps in the methods and uses of a device may be rearranged while remaining within the spirit and scope of the present disclosure. The accompanying claims present elements of the steps in a sample order, and are not meant to be limited to the specific order presented.

Unless otherwise stated, all measurements, values, ratings, positions, magnitudes, sizes, locations, orientations, configurations, and other specifications that are set forth (either expressly or impliedly) in this specification, including in the figures and in the claims that follow, are approximate, and not exact. They are intended to have a reasonable range that is consistent with the functions to which they relate and with what is customary in the art to which they pertain.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

### REFERENCES

1. “Aromatic.” The Oxford Pocket Dictionary of Current English. Retrieved Nov. 24, 2021 from Encyclopedia.com: https://www.encyclopedia.com/humanities/dictionaries-thesauruses-pictures-and-press-releases/aromatic-0
2. Abdelrahman B. Fadhil & Ligaa I. Saeed (2016) Sulfonated tea waste: A low-cost adsorbent for purification of biodiesel, International Journal of Green Energy, 13:1, 110-118, DOI: 10.1080/15435075.2014.896801
3. Alchimia Grow Shop. (2014, June 24). Marijuana terpenes and their effects. Alchimia Grow Shop. Retrieved Dec. 14, 2021, from https://www.alchimiaweb.com/blogen/marijuana-terpenes-effects/#Terpenesaromaticmo leculesinplants.
4. Andre, C. M., Hausman, J. F., & Guerriero, G. (2016). Cannabis sativa: The Plant of the Thousand and One Molecules. Frontiers in plant science, 7, 19. https://doi.org/10.3389/fpls.2016.00019
5. Angstrom-Tech-Admin (Apr. 20, 2016). “What's the Difference Between Positive and Negative Air Pressure Cleanrooms?”. Angstrom Technology.
6. Archetti, M., Döring, T. F., Hagen, S. B., Hughes, N. M., Leather, S. R., Lee, D. W., Lev-Yadun, S., Manetas, Y., Ougham, H. J., Schaberg, P. G., & Thomas, H. (2009). Unravelling the evolution of autumn colours: an interdisciplinary approach. Trends in ecology & evolution, 24(3), 166-173. https://doi.org/10.1016/i.tree.2008.10.006
7. Ashenhurst, J. (2020, February 12). Polar protic? polar aprotic? Nonpolar? all about solvents. Master Organic Chemistry. Retrieved Nov. 29, 2021, from https://www.masterorganicchemistry.com/2012/04/27/polar-protic-polar-aprotic-nonpol ar-all-about-solvents/.
8. Ashford's Dictionary of Industrial Chemicals, Third edition. p. 76.
9. Bennett, P. (2021, September 27). What is decarboxylation, and why does your cannabis need it? Leafly. Retrieved Nov. 29, 2021, from https://www.leafly.com/news/cannabis-101/what-is-decarboxylation.
10. Brenneisen, Rudolf (2007). Chapter 2: Chemistry and Analysis of Phytocannabinoids and Other Cannabis Constituents. In ElSohly, Mahmoud A. (ed.). Marijuana and the Cannabinoids. Totowa, NJ: Humana Press/Springer. pp. 17-49. ISBN 978-1-59259-947-9
11. Burstein S. H. (2014). The cannabinoid acids, analogs and endogenous counterparts. Bioorganic & medicinal chemistry, 22(10), 2830-2843. https://doi.org/10.1016/i.bmc.2014.03.038
12. Cannabinoid information—Montana. Montana State Legislator. (2010, August). Retrieved Dec. 7, 2021, from https://leg.mt.gov/content/Committees/Interim/2009_2010/Children_Family/Emerging-Issue/mmga-presentation-cannabinoid-information-aug2010.pdf.
13. Davies, Kevin M. (2004). Plant pigments and their manipulation. Wiley-Blackwell. p. 6. ISBN 978-1-4051-1737-1.
14. Dibble, C J., and Cole, IB. (Jan. 12, 2017). U.S. Pub. No. 2017/0008870A1. “Methods for Obtaining Purified Cannabis Extracts and THCA Crystals”
15. Dybing, E., Schwarze, P.E., Nafstad, P., Victorin, K., & Penning, T.M. (2015). POLYCYCLIC AROMATIC HYDROCARBONS IN AMBIENT AIR AND CANCER.
16. Eriksson, N., Wu, S., Do, C.B. et al. A genetic variant near olfactory receptor genes influences cilantro preference. Flavour 1, 22 (2012). https://doi.org/10.1186/2044-7248-1-22
17. Extract definition & information. Weedmaps. (2021, November 9). Retrieved Nov. 22, 2021, from https://weedmaps.com/learn/dictionary/extract.
18. Foucault, P., A. (1995). Centrifugal Partition Chromatography. New York: Marcel Dekker, Inc. ISBN 0-8247-9257-2
19. Gerber, F.; Krummen, M.; Potgeter, H.; Roth, A.; Siffrin, C.; Spoendlin, C. (2004). “Practical aspects of fast reversed-phase high-performance liquid chromatography using 3 μm particle packed columns and monolithic columns in pharmaceutical development and production working under current good manufacturing practice”. Journal of Chromatography A. 1036 (2): 127-133. doi:10.1016/j.chroma.2004.02.056
20. Gharib, Z., and Gharib, A. (Dec. 31, 2019). U.S. Pat. No. 10,517,911. “Manufacturing methods, compositions, and medical applications of orally administered cannabis pharmaceuticals using representative/total/complete cannabis extractions (cannabis infused pills)”
21. Grof CPL. Cannabis, from plant to pill. Br J Clin Pharmacol. 2018 Nov.; 84(11):2463-2467. doi: 10.1111/bcp.13618. Epub 2018 May 24. PMID: 29701252; PMCID: PMC6177712.
22. Harwood, L., M. and Moody, C., J. (1989). Experimental organic chemistry: Principles and Practice (Illustrated ed.). pp. 47-51
23. Helmenstine, A. M. (2020, September 2). Examples of polar and nonpolar molecules. ThoughtCo. Retrieved Nov. 29, 2021, from https://www.thoughtco.com/examples-of-polar-and-nonpolar-molecules-608516.
24. Hemp production. Hemp Production| Agricultural Marketing Service. (2018). Retrieved Dec. 7, 2021, from https://www.ams.usda.gov/rules-regulations/hemp.
25. Herkenroth, T., Steup, C. (Jun. 28, 2016). U.S. Pat. No. 9,376,367. “Cannabinoid carboxylic acids, salts of cannabinoid carboxylic acids, and the production and uses of same”
26. Industrial hemp. Department of Agriculture. (2021). Retrieved Dec. 9, 2021, from https://ag.colorado.gov/plants/industrial-hemp.
27. Jikomes, N. (2021, June 5). A list of major cannabinoids in cannabis and their effects. Leafly. Retrieved Nov. 19, 2021, from https://www.leafly.com/news/cannabis-101/list-major-cannabinoids-cannabis-effects.
28. Jürgen-Hinrich., Fuhrhop (2003). Organic synthesis: concepts and methods. Li, Guangtao, Dr. (3rd, completely rev. and enl. ed.). Weinheim: Wiley-VCH. p. 26. ISBN 9783527302727.
29. Laurain, N., and Zard, S. (Feb. 5, 2004). U.S. Pub. No. 2004/0024238 A1. “Use of nitriles as polar aprotic solvents”
30. Mayo, J. and Rieder, A. (Jun. 30, 2020). U.S. Pat. No. 10,696,646 “Purification of cannabinoids from crude cannabis oil”
31. Melnichuk, L. J., and Kelly S. K. (Apr. 21, 2020). U.S. Pat. No. 10,625,176. “Method for continuous extraction and separation of useful compounds for plant or animal material”
32. Olive, G., and Olive, S. (Dec. 18, 1979). U.S. Pat. No. 4,179,462. “Process for preparing acetonitrile”
33. Oraskar A., et al. (Nov. 1, 2020). TW Pub. No. 202039455A, “Process for purifying tetrahydrocannabinol using chromatographic stationary phase”
34. Oraskar A., et al. (Nov. 24, 2020). U.S. Pat. No. 10,843,991 “Process for separating a constituent/cannabinoid using a chromatographic resin”
35. Parekh, V. J., Rathod, V. K., & Pandit, A. B. (2011). Substrate hydrolysis. Comprehensive Biotechnology, 103-118. https://doi.org/10.1016/b978-O-08-088504-9.00094-5
36. Roura, N., X. (Feb. 19, 2019). U.S. Pat. No. 10,207,199 “Methods of purifying cannabinoids using liquid:liquid chromatography”
37. Russo, Taming THC: potential cannabis synergy and phytocannabinoid-terpenoid entourage effects, British J. Pharm., 163:1344-64 (2011).
38. Silberberg, Martin (2004). Chemistry: The Molecular Nature Of Matter and Change. New York: McGraw-Hill Companies. ISBN 0-07-310169-9.
39. Small D M, Green B G. A Proposed Model of a Flavor Modality. In: Murray M M, Wallace M T, editors. The Neural Bases of Multisensory Processes. Boca Raton (FL): CRC Press/Taylor & Francis; 2012. Chapter 36. Available from: https://www.ncbi.nlm.nih.gov/books/NBK92876/
40. Sommano, S. R., Chittasupho, C., Ruksiriwanich, W., & Jantrawut, P. (2020). The Cannabis Terpenes. Molecules (Basel, Switzerland), 25(24), 5792. https://doi.org/10.3390/molecules25245792
41. Sparks, T and Chase, G. (2015). Filters and Filtration Handbook (6th ed.). Butterworth-Heinemann. ISBN 9780080993966.
42. Specter M (Sep. 28, 2009). “A Life of Its Own”. The New Yorker.
43. Steiner, P. A.; Gordy, W. (1966). “Journal of Molecular Spectroscopy”. 21:291.
44. Svarovski, L. (2000). Solid-liquid separation. Butterworth-Heinemann.
45. Szalay, J. (2015, October 20). What are flavonoids? LiveScience. Retrieved Dec. 14, 2021, from https://www.livescience.com/52524-flavonoids.htmL.
46. Taura et al., Cannabidiolic-acid synthase, the chemotype-determining enzyme in the fiber-type Cannabis sativa, FEBS Letters, 581(16) pp. 2929-34 (2007).
47. Verzura, T., and Blackmon, E. (Apr. 28, 2016). PCT Pub. No. WO2016/064987A1, “Cannabis extracts and preparation and use methods thereof”
48. Wang, M., Wang, Y-H., Avula, B., Radwan, M. M., Wanas, A. S., van Antwerp, J., Parcher, J. F., ElSohly, M. A., &amp; Khan, I. A. (2016). Decarboxylation study of acidic cannabinoids: A novel approach using ultra-high-performance supercritical fluid chromatography/photodiode array-mass spectrometry. Cannabis and Cannabinoid Research, 1(1), 262-271. https://doi.org/10.1089/can.2016.0020
49. What is C18? A guide on the basics of HPLC columns. Develosil USA. (2019, November 6). Retrieved Dec. 14, 2021, from https://develosil.us/what-is-cl8-a-guide-on-the-basics-of-hplc-columns/.
50. Whittle, B., et al. (Feb. 19, 2004). U.S. Pat. No. 7,344,736. “Extraction of pharmaceutically active components from plant materials”
51. Why Does Alkyl Chain Length Matter? Cayman Chemical. (2020, April 13). Retrieved Dec. 7, 2021, from https://www.caymanchem.com/news/why-does-alkyl-chain-length-matter.

What is claimed is:

1. A method of facile purification of cannabinoid acids, the method comprising:
a. combining a cannabis extract and a polar aprotic solvent to form a solution;
b. mixing the solution to form a cannabinoid acid precipitate; and
c. obtaining the cannabinoid acid precipitate.

2. A method of facile purification of cannabinoid acids, the method comprising:
a. combining a cannabis extract and a polar aprotic solvent to form a solution;
b. mixing the solution to form a slurry;
c. filtering the slurry; and
d. obtaining a cannabinoid acid precipitate.

3. The method of either of claims 1 or 2, wherein the polar aprotic solvent is an organic nitrile solvent.

4. The method of claim 3, wherein the organic nitrile solvent is selected from the group consisting of acetonitrile, acrylonitrile, benzonitrile, butyronitrile, decanenitrile, pivalonitrile, propionitrile, butyronitrile, isobutyronitrile, malononitrile, and succinonitrile.

5. The method of claim 4, wherein the organic nitrile solvent is acetonitrile.

6. The method of claim 3, wherein the organic nitrile solvent has a melting point below 40° C.

7. The method of claim 3, wherein the organic nitrile solvent has a boiling point below 180° C.

8. The method of claim 7, wherein the organic nitrile solvent has a boiling point between 70° C. and 150° C.

9. The method of claim 3, wherein the organic nitrile solvent has a dielectric constant above 15.0.

10. The method of claim 9, wherein the organic nitrile solvent has a dielectric constant above 37.0.

11. The method of claim 3, wherein the organic nitrile solvent has a density between 0.75 and 1.40 g/cm³.

12. The method of claim 3, wherein the organic nitrile solvent has a dipole moment above 0.1 D.

13. The method of claim 12, wherein the organic nitrile solvent has a dipole moment between 1.8 D and 3.6 D.

14. The method of claim 12, wherein the organic nitrile solvent has a dipole moment between 3.2 D and 3.4 D.

15. The method of claim 3, wherein the cannabis extract was extracted by any of supercritical CO₂, subcritical CO₂, an alcohol, or a hydrocarbon.

16. The method of claim 3, wherein the cannabis extract has an initial purity of less than 60%.

17. The method of claim 3, wherein the cannabis extract has an initial purity of at least 60%.

18. The method of claim 3, wherein the cannabis extract was extracted from an industrial hemp plant material that contained no more than 0.3% THC on a dry-weight basis.

19. The method of either of claims 1 or 2, further comprising the step of purifying the cannabis extract before combining the cannabis extract and the polar aprotic solvent.

20. The method of claim 19, wherein the purification is completed through the use of HPLC, with a column comprising an adsorbent selected from the group consisting of activated carbon, silica gel, alumina, bentonite, and acid activated bleaching clay.

21. The method of claim 19, wherein the purification is completed through the use of winterization or dewaxing.

22. The method of claim 21, wherein the cannabis extract is winterized using the steps comprising:
a. dissolving the cannabis extract in an alcohol solvent;
b. chilling the cannabis extract and the alcohol solvent to between about 0° C. to about −60° C.;
c. filtering the cannabis extract and the alcohol solvent to yield a filtrate; and
d. optionally, distilling the filtrate at a temperature below about 65° C. so as to substantially remove the alcohol solvent.

23. The method of claim 22, wherein the cannabis extract and the alcohol solvent are chilled to between about −5° C. and about −45° C.

24. The method of either of claims 1 or 2, wherein the cannabis extract does not undergo an initial purification step of purifying the cannabis extract before combining the cannabis extract and the polar aprotic solvent.

25. The method of either of claims 1 or 2, wherein the ratio of polar aprotic solvent to cannabis extract is between about 300 mL to about 600 mL of the polar aprotic solvent for every about 1,000 g of cannabis extract.

26. The method of either of claims 1 or 2, wherein the ratio of polar aprotic solvent to cannabis extract is between about 400 mL to about 600 mL of the polar aprotic solvent for every about 1,000 g of cannabis extract.

27. The method of either of claims 1 or 2, wherein the ratio of polar aprotic solvent to cannabis extract is between about 350 mL to about 450 mL of the polar aprotic solvent for every about 1,000 g of cannabis extract.

28. The method of either of claims 1 or 2, further comprising the step of evaporating residual solvent.

29. The method of claim 28, wherein the residual solvent is evaporated through the use of ambient evaporation.

30. The method of claim 29, wherein the ambient evaporation occurs at temperatures between about 15° C. to about 35° C.

31. The method of claim 28, wherein the residual solvent is evaporated through the use of a vacuum oven.

32. The method of claim 31, wherein the vacuum oven is heated to between about 25° C. to about 45° C., and maintained at a pressure of less than about 200 torr.

33. The method of claim 28, wherein the residual solvent is evaporated through the use of a rotary evaporator and a water bath.

34. The method of claim 33, wherein the water bath is heated to a temperature of between about 65° C. and 70° C., has a beginning pressure of between about 400 torr to about 500 torr, and has a final pressure of between about 5 torr to about 30 torr.

35. The method of claim 2, wherein the filtering step is performed using any of gravity filtration, vacuum filtration, pressure filtration, and centrifugal filtration.

36. The method of claim 2, wherein the filtering step is performed using a filter having a first filter screen and a second filter screen, wherein the first filter screen contains a cut size of between about 110 microns to about 140 microns, and the second filter screen contains a pore size of between about 70 microns to about 100 microns.

37. The method of either of claims 1 or 2, wherein the mixing is performed by a magnetic stir bar or high-shear blade.

38. The method of claim 37, wherein the magnetic stir bar or high-shear blade operates at a stirring speed of between about 300 rpm to about 1,500 rpm.

39. The method of either of claims 1 or 2, wherein the mixing is performed until the slurry is substantially homogenous.

40. The method of claim 1, wherein the mixing is performed until the cannabinoid acid precipitate has substantially ceased precipitating.

41. The method of claim 2, wherein the mixing is performed until the slurry is substantially homogenous.

42. The method of claim 41, wherein the mixing is performed by agitating the solution until the slurry is substantially homogenous, and the cannabinoid acid precipitate has substantially ceased precipitating.

43. The method of either of claims 1 or 2, wherein the mixing is performed for between about 10 minutes to about 36 hours.

44. The method of claim 43, wherein the mixing is performed for between about 2 hours to about 24 hours.

45. The method of claim 44, wherein the mixing is performed for between about 6 hours to about 18 hours.

46. The method of either of claims 1 or 2, wherein the mixing is performed at a temperature of between about 40° C. to about 80° C.

47. The method of either of claims 1 or 2, wherein the cannabinoid acid precipitate comprises at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

48. The method of claim 47, wherein the cannabinoid acid precipitate has a purity of at least 99% cannabinoid acids.

49. The method of claim 47, wherein the cannabinoid acid precipitate comprises THCA.

50. The method of claim 49, wherein the cannabinoid acid precipitate has a purity of at least 99% THCA.

51. The method of claim 48, wherein the cannabinoid acid precipitate has no more than 5% THCA.

52. The method of claim 3, wherein the cannabinoid acid precipitate comprises at least one of CBCA, CBCVA, CBDA, CBDMA, CBDPA, CBDVA, CBEA, CBFA, CBGA, CBGVA, CBLA, CBNA, CBNDA, CBTA, THCA, THCPA, and THCVA.

53. The method of claim 52, wherein the cannabinoid acid precipitate has a purity of at least 99% cannabinoid acids.

54. The method of claim 52, wherein the cannabinoid acid precipitate comprises THCA.

55. The method of claim 54, wherein the cannabinoid acid precipitate has a purity of at least 99% THCA.

56. The method of claim 53, wherein the cannabinoid acid precipitate has no more than 5% THCA.

57. The method of either of claims 1 or 2, further comprising the step of decarboxylating the cannabinoid acid precipitate.

58. The method of claim 57, wherein the decarboxylation step is performed by heating the cannabinoid acid precipitate to a temperature of between about 70° C. to about 200° C.

59. The method of claim 58, wherein the cannabinoid acid precipitate is heated to a temperature of between about 85° C. to about 150° C. under ambient pressure.

60. The method of claim 58, wherein the cannabinoid acid precipitate is heated to a temperature of between about 70° C. to about 120° C. under vacuum.

61. A cannabinoid acid precipitate having a purity of at least 99% cannabinoid acids, wherein the cannabinoid acid precipitate is prepared according to the method of either of claims 1 or 2.

62. The cannabinoid acid precipitate of claim 61, having a purity of at least 99% THCA.

63. The cannabinoid acid precipitate of claim 61, having no more than 5% THCA.

64. A cannabinoid acid precipitate having a purity of at least 99% cannabinoid acids, wherein the cannabinoid acid precipitate is prepared according to the method of claim 3.

65. The cannabinoid acid precipitate of claim 64, having a purity of at least 99% THCA.

66. The cannabinoid acid precipitate of claim 64, having no more than 5% THCA.

67. The method of claim 2, further comprising the steps of: (a) collecting a mother liquor in a container when filtering the slurry; (b) cooling the mother liquor to between about −10° C. to about −40° C.; (c) waiting until any dark pigments settle in the container; and (d) pouring out the mother liquor, to obtain a lighted solution.
